(19) United States
(12) Patent Application Publication
(10) Pub. No.: US 2024/0368294 A1
(43) Pub. Date: Nov. 7, 2024

(54) CD40L-SPECIFIC TN3-DERIVED SCAFFOLDS FOR USE IN THE TREATMENT AND PREVENTION OF RHEUMATOID ARTHRITIS

(71) Applicant: Viela Bio, Inc., Deerfield, IL (US)

(72) Inventors: Gabor G. ILLEI, Rockville, MD (US); Ilias ALEVIZOS, Edgewater, NJ (US); Liangwei WANG, North Potomac, MD (US); Judith FALLOON, Bethesda, MD (US); William A. REES, North Bethesda, MD (US)

(21) Appl. No.: 18/615,679

(22) Filed: Mar. 25, 2024

Related U.S. Application Data
(63) Continuation of application No. PCT/US2022/077192, filed on Sep. 28, 2022.
(60) Provisional application No. 63/249,552, filed on Sep. 28, 2021.
(60) Provisional application No. 63/322,379, filed on Mar. 22, 2022.
(60) Provisional application No. 63/337,274, filed on May 2, 2022.

(51) Int. Cl. C07K 16/28; A61K 9/00; A61K 39/395; A61K 45/06; A61P 19/02; C07K 14/765

(52) U.S. Cl. CPC C07K 16/2875; A61K 9/0019; A61K 39/3955; A61K 45/06; A61P 19/02; C07K 14/765; C07K 2318/20

(57) ABSTRACT

A human CD40L-specific Tn3 molecule and therapeutic uses thereof for the treatment of autoimmune disease (e.g., rheumatoid arthritis).

### CROSS-REFERENCE TO RELATED APPLICATIONS

The present application is a continuation of International Application No. PCT/US2022/077192, filed Sep. 28, 2022, which claims priority to U.S. Provisional Patent Application No. 63/337,274, filed May 2, 2022, U.S. Provisional Patent Application No. 63/322,379, filed Mar. 22, 2022, and U.S. Provisional Patent Application No. 63/249,552, filed Sep. 28, 2021, each of which is incorporated by reference herein in their entirety for all purposes.

### REFERENCE TO AN ELECTRONIC SEQUENCE LISTING

The contents of the electronic sequence listing (HOPA_034_03US_SeqList_ST26.xml; Size: 24,489 bytes; and Date of Creation: Mar. 25, 2024) are herein incorporated by reference in its entirety.

### TECHNICAL FIELD

The present disclosure is related to compositions comprising a Tn3 scaffold and methods using the same in the treatment prevention of rheumatoid arthritis.

### BACKGROUND

Rheumatoid arthritis (RA) is a chronic systemic inflammatory disease that is associated with significant morbidity and mortality. The disease is characterized by inflammation of the synovial joints that can result in pain, swelling, and joint damage with secondary deformity and progressive disability. Worldwide, the prevalence of RA is estimated to be between 0.6-1.1% with variations across geographical regions. The incidence is 2-3 times higher in women than in men with a peak age of onset between 35-55 years of age. Uncontrolled active RA causes joint damage, disability, and decreased quality of life; comorbidities include cardiovascular disease and osteoporosis, and reduced life expectancy.

Despite the presence of existing therapeutic agents for the treatment of RA, there is a need for new treatments to reduce disease activity, because only a minority of subjects achieve clinical remission.

### BRIEF SUMMARY

Provided are methods for treating rheumatoid arthritis in a subject in need thereof comprising administering a Tn3 scaffold comprising a CD40L-specific monomer subunit to the subject. In aspects, the Tn3 scaffold specifically binds to CD40L. In aspects, the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, wherein the Tn3 scaffold comprising the CD40L-specific monomer subunit is administered at a dose of about 1500 mg, and wherein the Tn3 scaffold is administered once about every 2 weeks for at least 2 doses, and is administered about once a month thereafter.

Provided are methods of treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 3 doses, and is administered about once a month thereafter.

Provided are methods of treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 months for at least 2 doses.

Provided are methods for treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 3000 mg, and wherein the Tn3 scaffold is administered about once, once a month, once about every two months, or once about every three months.

In aspects, the Tn3 scaffold is administered in combination with a second therapy. In aspects, the second therapy comprises an anti-inflammatory agent, an anti-pain agent, a Disease-modifying Antirheumatic Drug (DMARD), a corticosteroid, or an immune system modifying agent. In aspects, the DMARD is a conventional DMARD (cDMARD) or a biologic DMARD (bDMARD). In aspects, the DMARD is the cDMARD, and wherein the cDMARD is selected from the group consisting of: hydroxychloroquine, leflunomide, sulfasalazine, tofacitinib, and combinations thereof. In aspects, the DMARD is the bDMARD, and wherein the bDMARD is selected from the group consisting of: etanercept, infliximab, adalimumab, certolizumab, and golimumab. In aspects, the Tn3 scaffold is administered intravenously. In aspects, the intravenous administration comprises an infusion. In aspects, the Tn3 scaffold comprises two CD40L-specific monomer subunits connected in tandem. In aspects, the Tn3 scaffold binds CD40L and prevents binding of CD40L to CD40 and/or disrupts CD40 mediated signaling. In aspects, at least one CD40L-specific monomer subunit is fused or conjugated to a heterologous moiety selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, and a cytokine. In aspects, at least one CD40L-specific monomer subunit is conjugated to PEG or is fused to HSA. In aspects, the HSA is a variant HSA comprising the amino acid sequence of SEQ ID NO: 4. In aspects, the Tn3 scaffold comprises the sequence of SEQ ID NO: 1. In aspects, a first dose of the Tn3 scaffold is administered at about 13 to about 25 mg/min, a second dose and third dose are administered at about 25 mg/min, and a fourth dose is administered at about 17 to about 33 mg/min. In aspects, a first dose of the Tn3 scaffold is administered at about 8 to about 17 mg/min, and a second dose and third dose are administered at about 13 mg/min. In aspects, a first dose of the Tn3 scaffold is about 1500 mg and subsequent doses are about 1500 mg or about 3000 mg. In aspects, the first and a second dose of the Tn3 scaffold are about 1500 mg and the subsequent doses are about 3000 mg. In aspects, a first dose is administered over 120 min or 180 min and subsequent doses are administered over 60 or 90 min. In aspects, a Tn3 scaffold is administered once. In aspects, a Tn3 scaffold is administered quarterly. In aspects, a quarterly administration of the Tn3 scaffold is equally efficacious or more efficacious than more frequent administrations of the Tn3 scaffold as determined by change from baseline in DAS28-CRP in a treated subject. In aspects, the change from baseline of the DAS28-CRP is determined quarterly. In aspects, the change from baseline of the DAS28-CRP is determined monthly. In aspects, the change from baseline of the DAS28-CRP is determined yearly. In aspects, the quarterly administration of the Tn3 scaffold confers sustained treatment efficacy in the subject in need as compared to an otherwise comparable subject undergoing more frequent administrations of the Tn3 scaffold as determined by a treatment assessment, and wherein the sustained treatment efficacy is of at least or at most about 15 days, 1 month, 2 months, 3 months, 4 months, 5 months, 6 months, 8 months, 10 months, 1 year, or 1.5 years. In aspects, a treatment assessment is determined quarterly.

Provided herein are methods for treating RA in a subject in need thereof comprising administering a Tn3 scaffold comprising a CD40L-specific monomer subunit to the subject. In aspects, the Tn3 scaffold specifically binds to CD40L. In aspects, the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16. In aspects, the Tn3 scaffold comprising the CD40L-specific monomer subunit is administered at a dose of about 1500 mg. In aspects, the Tn3 scaffold is administered once about every 2 weeks for at least 2 doses, and is administered about once a month thereafter.

Provided herein are methods for treating rheumatoid arthritis in a subject in need thereof comprising administering a Tn3 scaffold comprising a CD40L-specific monomer subunit to the subject. In aspects, the Tn3 scaffold specifically binds to CD40L. In aspects, the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16. In aspects, the Tn3 scaffold comprising the CD40L-specific monomer subunit is administered at a dose of about 1500 mg. In aspects, the Tn3 scaffold is administered once about every 2 weeks for at least 3 doses, and is administered about once a month thereafter.

Provided herein are methods for treating rheumatoid arthritis in a subject in need thereof comprising administering a Tn3 scaffold comprising a CD40L-specific monomer subunit to the subject. In aspects, the Tn3 scaffold specifically binds to CD40L. In aspects, the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16. In aspects, the Tn3 scaffold comprising the CD40L-specific monomer subunit is administered at a dose of about 1500 mg. In aspects, the Tn3 scaffold is administered once about every 2 months for at least 2 doses.

Provided herein are methods for treating rheumatoid arthritis in a subject in need thereof comprising administering a Tn3 scaffold comprising a CD40L-specific monomer subunit to the subject. In aspects, the Tn3 scaffold specifically binds to CD40L. In aspects, the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16. In aspects, the Tn3 scaffold comprising the CD40L-specific monomer subunit is administered at a dose of about 3000 mg. In aspects, the Tn3 scaffold is administered about once a month, once about every two months, or once about every three months.

These and other aspects are described below.

### BRIEF DESCRIPTION OF THE DRAWINGS

The accompanying figures, which are incorporated herein and form a part of the specification, illustrate some, but not the only or exclusive, example aspects and/or features. It is intended that the aspects and figures disclosed herein are to be considered illustrative rather than limiting.

FIG. 1 shows an exemplary study flow diagram. The Tn3 scaffold is VIB4920. VIB4920 comprises a bivalent CD40L-specific Tn3 protein fused to a human serum albumin (HSA) and has the sequence shown in SEQ ID NO:1. Approximately 75 subjects randomized (1:1:1:1:1), placebo-controlled, parallel design. All subjects are dosed 4 times total, with each dose administered on days 1, 15, 29 and 57, and doses that are not VIB4920 are placebo. Study population comprises adults (≥18 years of age) with active, moderate-to-severe adult-onset RA (e.g., DAS28-CRP>3.2; >4 tender and >4 swollen joints) and presence of serum rheumatoid factor (RF) and/or anti-citrullinated protein antibodies (ACPAs) with inadequate response to Methotrexate (MTX), cDMARD, or anti-TNFα agent and no prior treatment with rituximab or B cell depletive agents.

FIG. 2 shows the change from baseline in DAS28-CRP in placebo, VIB4920 3000 mg×1, VIB4920 1500 mg×2, VIB4920 3000 mg×2, and VIB4920 1500 mg×4 groups.

FIG. 3 shows the change from baseline in CDAI in placebo, VIB4920 3000 mg×1, VIB4920 1500 mg×2, VIB4920 3000 mg×2, and VIB4920 1500 mg×4 groups. Summary statistics for day 113 are listed.

FIG. 4 shows the change from baseline in SDAI in placebo, VIB4920 3000 mg×1, VIB4920 1500 mg×2, VIB4920 3000 mg×2, and VIB4920 1500 mg×4 groups. Summary statistics for day 113 are listed.

FIG. 5 shows the change from baseline in Swollen Joint Count in placebo, VIB4920 3000 mg×1, VIB4920 1500 mg×2, VIB4920 3000 mg×2, and VIB4920 1500 mg×4 groups. Summary statistics for day 113 are listed.

FIG. 6 shows the change from baseline in Tender Joint Count in placebo, VIB4920 3000 mg×1, VIB4920 1500 mg×2, VIB4920 3000 mg×2, and VIB4920 1500 mg×4 groups. Summary statistics for day 113 are listed.

FIG. 7 shows the change from baseline in Physician Global Assessment in placebo, VIB4920 3000 mg×1, VIB4920 1500 mg×2, VIB4920 3000 mg×2, and VIB4920 1500 mg×4 groups. Summary statistics for day 113 are listed.

FIG. 8 shows the change from baseline in Patient Global Assessment in placebo, VIB4920 3000 mg×1, VIB4920 1500 mg×2, VIB4920 3000 mg×2, and VIB4920 1500 mg×4 groups.

FIG. 9 shows the change from baseline in CRP in placebo, VIB4920 3000 mg×1, VIB4920 1500 mg×2, VIB4920 3000 mg×2, and VIB4920 1500 mg×4 groups.

FIG. 10 shows the change from baseline in pain in placebo, VIB4920 3000 mg×1, VIB4920 1500 mg×2, VIB4920 3000 mg×2, and VIB4920 1500 mg×4 groups.

FIG. 11 shows the change from baseline in HAQ in placebo, VIB4920 3000 mg×1, VIB4920 1500 mg×2, VIB4920 3000 mg×2, and VIB4920 1500 mg×4 groups.

FIG. 12 shows the change from baseline in FACIT-Fatigue in placebo, VIB4920 3000 mg×1, VIB4920 1500 mg×2, VIB4920 3000 mg×2, and VIB4920 1500 mg×4 groups.

FIG. 13 shows the proportion of subjects with clinical remission (DAS28-CRP≤2.6) in placebo, VIB4920 3000 mg×1, VIB4920 1500 mg×2, VIB4920 3000 mg×2, and VIB4920 1500 mg×4 groups.

FIG. 14 shows the proportion of subjects with clinical remission (CDAI≤2.8) in placebo, VIB4920 3000 mg×1, VIB4920 1500 mg×2, VIB4920 3000 mg×2, and VIB4920 1500 mg×4 groups.

FIG. 15 shows the proportion of subjects with low disease activity as determined by DAS28-CRP≤3.2 and an improvement of DAS28-CRP score>0.6 from baseline.

FIG. 16 shows the time (days) to start of rescue medication in placebo, VIB4920 3000 mg×1, VIB4920 1500 mg×2, VIB4920 3000 mg×2, and VIB4920 1500 mg×4 groups.

FIG. 17 shows the change from baseline CXCL13 in placebo, VIB4920 3000 mg×1, VIB4920 1500 mg×2, VIB4920 3000 mg×2, and VIB4920 1500 mg×4 groups.

FIG. 18 shows the soluble CD40L in placebo, VIB4920 3000 mg×1, VIB4920 1500 mg×2, VIB4920 3000 mg×2, and VIB4920 1500 mg×4 groups.

FIG. 19 shows the change from baseline in RF in placebo, VIB4920 3000 mg×1, VIB4920 1500 mg×2, VIB4920 3000 mg×2, and VIB4920 1500 mg×4 groups. Summary statistics for day 113 are listed. BSL=baseline; PBO=placebo.

FIG. 20A-FIG. 20C show the change from baseline in RF Isotypes. FIG. 20A shows RF IgA.

FIG. 20B shows RF IgG. FIG. 20C shows RF IgM. All changes from baseline depict in placebo, VIB4920 3000 mg×1, VIB4920 1500 mg×2, VIB4920 3000 mg×2, and VIB4920 1500 mg×4 groups.

FIG. 21 shows an exemplary study schematic. From week 12 onward, subjects with persistent or worsening disease activity (including those whose RA improved and subsequently worsened) may receive additional treatment to manage their RA at the discretion of their rheumatologist. Subjects whose treatment for RA is changed or increased due to persistent or worsening disease activity is followed through week 40 according to an alternate monitoring schedule, see Table 25.

FIG. 22 is a graphic showing joints assessed in a 44 and 28 joint count. The following joints are to be assessed for tenderness and swelling in the 44-joint count, as shown in with gray circles: sternoclavicular (2), acromioclavicular (2), shoulder (2), elbow (2), wrist (2), metacarpophalangeal (10), finger proximal interphalangeal (10), knee (2), ankle (2), and metatarsophalangeal (10) joints. Out of these 44 joints, assessment of the 28 joints indicated by X's on the figure: shoulder (2), elbow (2), wrist (2), metacarpophalangeal (10), finger proximal interphalangeal (10), and knee (2) joints will be used to calculate SDAI and DAS28-CRP.

FIG. 23A-FIG. 23B depict RAPID analysis and categories. FIG. 23A is a graphic showing the RAPID3 analysis. FIG. 23B shows categories of disease severity defined by the RAPID3. The RAPID3 analysis is obtained from rheumatology.org/Portals/0/Files/RAPID3%20.

FIG. 24 shows the Health Assessment Questionnaire Disability Index (HAQ-DI). The questionnaire is obtained from Stanford Health Assessment Questionnaire: Dimensions and Practical Applications.

FIG. 25 shows the PROMIS-29 profile. The profile is obtained from 2008-2013 PROMIS Health Organization and PROMIS Cooperative Group.

FIG. 26 shows percent improvement from baseline needed to achieve ACR20, ACR50, ACR70 response.

FIG. 27 shows percent of subjects with serious infections in RA trials using two biologic agents.

### DETAILED DESCRIPTION

Provided herein are Tn3 scaffolds that are anti-cluster of differentiation (CD) 40 ligand (CD40L)—third fibronectin type III (Fn3) protein domain of human Tenascin C (Tn3) protein fusion proteins and methods of using the same in autoimmune disease. In aspects, compositions and methods provided are utilized for the treatment of B-cell dependent autoimmune diseases, such as rheumatoid arthritis (RA). Genome-wide association studies have identified a common variant in the CD40 locus that increases the risk of RA. The expression of CD40L on CD4+ T helper cells is also increased in subjects with active RA compared to healthy controls. Taken together, these observations suggest that inhibition of the CD40L/CD40 pathway may be beneficial in RA.

Also provided are methods comprising administering Tn3 scaffolds with TNF-α inhibitors (TNFi) for the treatment of autoimmune disease.

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the” include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “subject” are used interchangeably herein. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile; fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, “treating” or “treat” describes the management and care of a subject for the purpose of combating a disease, condition, or disorder and includes the administration of a Tn3 scaffold used in the methods described herein to alleviate the symptoms or complications of a disease, condition or disorder, or to eliminate the disease, condition or disorder. Thus, the term “treat” or “treating” refers to both therapeutic measures and prophylactic or preventative measures, wherein the objective is to prevent, slow down (lessen), or ameliorate the progression of a disease (e.g., RA). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

As used herein, “fused” refers to at least two polypeptides joined recombinantly. As used herein “conjugated” refers to formation of a bond between two components by chemical reaction. The bond may be covalent or non-covalent. Typically, two components that are conjugated to each other are chemically connected via a covalent bond.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

### Overview

Described herein are methods for treating an autoimmune disorder using a Tn3 scaffold comprising a CD40L-specific monomer subunit. Also provided are compositions and methods of combining a Tn3 scaffold with a secondary therapy such as a TNFi.

In aspects, the Tn3 scaffold is used in methods of treating rheumatoid arthritis (RA). RA is a chronic systemic inflammatory disease that is associated with significant morbidity and mortality. RA comprises inflammation of the synovial joints that can result in pain, swelling, and joint damage with secondary deformity and progressive disability. In aspects, the methods comprise treating RA in a subject in need thereof by administering the Tn3 scaffold comprising a CD40L-specific monomer subunit. In aspects, the Tn3 scaffold specifically binds to CD40L. In aspects, the monomer subunit of the Tn3 scaffold comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16. In aspects, a Tn3 scaffold comprises or consists of SEQ ID NO: 1.

The CD40 receptor is a member of the TNF family of receptors expressed on the plasma membrane of antigen-stimulated B cells, macrophage, and dendritic cells. The CD40 receptor functions to provide a co-stimulatory signal for B cells that have bound antigen. The cognate ligand for CD40 is CD40L (also known as CD154), which is expressed on the plasma membrane of T cells and other cell types, including platelets.

In aspects, subjects with RA comprise a common variant in the CD40 locus that increases the risk of RA. The RA risk allele is a gain-of-function allele that increases the amount of CD40 on the surface of at least primary human B-lymphocyte cells. In aspects, expression of CD40L on CD4+T helper cells is also increased in subjects with active RA compared to control subjects. Taken together, these observations suggest that inhibition of the CD40L/CD40 pathway may be beneficial in RA.

### Tn3 Scaffolds Comprising a CD40L-Specific Monomer Subunit

Provided herein are compositions that bind CD40L. In aspects, provided compositions comprise CD40L antagonists. In aspects, provided herein are compositions that comprise a Tn3 scaffold comprising a CD40L-specific monomer subunit (e.g., “Tn3 scaffold”). In aspects, provided herein are compositions that comprise a Tn3 scaffold comprising two CD40L-specific monomer subunits.

In aspects, a CD40L monomer subunit of a Tn3 scaffold comprises seven beta strands designated A, B, C, D, E, F, and G, and six loop regions designated AB, BC, CD, DE, EF, and FG. In aspects, the Tn3 scaffold comprises a single CD40L-specific monomer subunit. In aspects, the Tn3 scaffold comprises two CD40L-specific monomer subunits. In aspects, the two CD40L-specific monomer subunits are connected in tandem. In aspects, the two CD40L-specific monomer subunits are connected by a linker. In other aspects, the linker comprises a peptide linker, which can be a flexible peptide linker. In aspects, the peptide linker comprises a (GₘX)ₙ sequence wherein X is Serine (S), Alanine (A), Glycine (G), Leu (L), Isoleucine (I), or Valine (V); m and n are integer values; m is 1, 2, 3 or 4; and, n is 1, 2, 3, 4, 5, 6, or 7.

In aspects, the Tn3 scaffold comprises a linker which comprises a functional moiety. In aspects, this functional moiety is an immunoglobulin or a fragment thereof. In aspects, this immunoglobulin or fragment thereof comprises an Fc domain. In aspects, this Fc domain fails to induce at least one FcγR-mediated effector function (e.g., Fc-deficient). In aspects, this at least one FcγR-mediated effector function is antibody-dependent cellular cytotoxicity (ADCC).

In aspects, the Tn3 scaffold comprises seven beta strands designated A, B, C, D, E, F, and G, and six loop regions designated AB, BC, CD, DE, EF, and FG, wherein the AB loop comprises or consists of SEQ ID NO: 11, the BC loop comprises or consists of SEQ ID NO: 12, the CD loop comprises or consists of SEQ ID NO: 13, the DE loop comprises or consists of SEQ ID NO: 14, the EF loop comprises or consists of SEQ ID NO: 15, and the FG loop comprises or consists of SEQ ID NO: 16. In aspects, the Tn3 scaffold comprises or consists of SEQ ID NO: 1. In aspects, beta strand A comprises or consists of SEQ ID NO: 5, beta strand B comprises or consists of SEQ ID NO: 6, beta strand C comprises or consists of SEQ ID NO: 17, beta strand D comprises or consists of SEQ ID NO: 18, beta strand E comprises or consists of SEQ ID NO: 19, beta strand F comprises or consists of SEQ ID NO: 20, and beta strand G comprises or consists of SEQ ID NO: 21.

In aspects, one or more CD40L-specific Tn3 monomers have a beta strand A comprising or consisting of IEV (SEQ ID NO: 5), RLDAPSQIEV (SEQ ID NO: 23), or SQIEV (SEQ ID NO: 24). In aspects, a Tn3 scaffold may comprise one or more CD40L-specific Tn3 monomers having the same or different beta strand A sequences. For example, a first CD40L-specific Tn3 monomer beta strand A may comprise or consist of IEV (SEQ ID NO: 5) and a second CD40L-specific Tn3 monomer beta strand A may comprise or consist of RLDAPSQIEV (SEQ ID NO: 23) or SQIEV (SEQ ID NO: 24).

The Tn3 scaffold may have the amino acid sequence as shown in SEQ ID NO: 1 and described above or it may have one or more amino acid residues changes relative to the amino acid sequence as shown in SEQ ID NO: 1. For example, if the Tn3 scaffold has amino acid sequence changes relative to those shown in SEQ ID NO: 1, the changes may be to one of the linkers. The Tn3 scaffold comprises a Gly15 linker separating two CD40L-specific monomers and a Gly10 linker separating a CD40L-specific monomer from an HSA sequence. Both or one of these linkers may be altered, and may be replaced with an amino acid sequence of (GₘX)ₙ wherein X is Serine (S), Alanine (A), Glycine (G), Leu (L), Isoleucine (I), or Valine (V); m and n are integer values; m is 1, 2, 3 or 4; and, n is 1, 2, 3, 4, 5, 6, or 7. For example, one or both linkers may be altered to have an amino acid sequence that comprises one of GGGGSGGGGS (SEQ ID NO: 7), GGGGSGGGGSGGGGS (SEQ ID NO: 8), GGGGGGGGGG (SEQ ID NO: 9) or GGGGGGGGGGGGGGG (SEQ ID NO: 10). If the Tn3 scaffold has an amino acid sequence relative to the amino acid sequence as provided in SEQ ID NO: 1, it may be due to a changes or changes in the HSA amino acid sequence fused to the two CD40L-specific monomers. The HSA fused to the two CD40L-specific monomers may be altered to relative to the HSA fused to the two CD40L-specific Tn3 monomers, except for at least one amino acid substitution, numbered relative to the position in full length mature HSA, at a position selected from the group consisting of 407, 415, 463, 500, 506, 508, 509, 511, 512, 515, 516, 521, 523, 524, 526, 535, 550, 557, 573, 574, and 580; wherein the at least one amino acid substitution does not comprise a lysine (K) to glutamic acid (E) at position 573.

Exemplary sequences for a Tn3 scaffold are shown in Table 1. In aspects, a Tn3 scaffold comprises at least about or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 25 shown in Table 1. In aspects, any one of the sequences from Table 1 can be modified. In aspects, a modification comprises one or more truncations, deletions, insertions, and combinations thereof. A modification can occur at any of the residues provided in Table 1 and in any number of residues from Table 1. In aspects, a modification can comprise from 1-3, 1-5, 1-10, 1-20, 3-8, 3-10, 3-15, 5-8, 5-10, or 5-20 residues. In aspects, a modification can occur in up to 1, 2, 3, 4, 5, 6, 7, 8, 9, 10, 11, 12, 13, 14, 15, 20, 30, 40, 50, 60, 70, 80, 90,100,200, 300, 400, or 450 residues.

TABLE 1
Exemplary sequences for a Tn3 scaffold comprising
a CD40L-specific monomer subunit

| SEQ | | |
|---|---|---|
| ID NO | ID | Sequence |
| 1 | VIB4920 | SQIEVKDVTDTTALITWSDDFGEYVWCELTYGIKDVPGDRTTIDL |
| | (342-G15-342- | WYHHAHYSIGNLKPDTEYEVSLICRSGDMSSNPAKETFTTGGGGG |
| | G10-HSAC34S | GGGGGGGGGGRLDAPSQIEVKDVTDTTALITWSDDFGEYVWCELT |
| | Bivalent | YGIKDVPGDRTTIDLWYHHAHYSIGNLKPDTEYEVSLICRSGDMS |
| | construct 2- | SNPAKETFTTGGGGGGGGGGDAHKSEVAHRFKDLGEENFKALVLI |
| | All GLY linkers | AFAQYLQQSPFEDHVKLVNEVTEFAKTCVADESAENCDKSLHTLF |
| | HSA | GDKLCTVATLRETYGEMADCCAKQEPERNECFLQHKDDNPNLPRL |
| | underlined) | VRPEVDVMCTAFHDNEETFLKKYLYEIARRHPYFYAPELLFFAKR |
| | | YKAAFTECCQAADKAACLLPKLDELRDEGKASSAKQRLKCASLQK |
| | | FGERAFKAWAVARLSQRFPKAEFAEVSKLVTDLTKVHTECCHGDL |
| | | LECADDRADLAKYICENQDSISSKLKECCEKPLLEKSHCIAEVEN |
| | | DEMPADLPSLAADFVESKDVCKNYAEAKDVFLGMFLYEYARRHPD |
| | | YSVVLLLRLAKTYETTLEKCCAAADPHECYAKVFDEFKPLVEEPQ |
| | | NLIKQNCELFEQLGEYKFQNALLVRYTKKVPQVSTPTLVEVSRNL |
| | | GKVGSKCCKHPEAKRMPCAEDYLSVVLNQLCVLHEKTPVSDRVTK |
| | | CCTESLVNRRPCFSALEVDETYVPKEFNAETFTFHADICTLSEKE |
| | | RQIKKQTALVELVKHKPKATKEQLKAVMDDFAAFVEKCCKADDKE |
| | | TCFAEEGKKLVAASQAALGL |
| 2 | CD40L-specific | IEVKDVTDTTALITWSDDFGEYVWCELTYGIKDVPGDRTTIDLWY |
| | Tn3 monomer | HHAHYSIGNLKPDTEYEVSLICRRGDMSSNPAKETFTT |
| | with affinity | |
| | mature variant | |
| | Clone 342- | |
| | Affinity mature | |
| | variant | |
| | (W/WT FG | |
| | loop; W/O N- | |
| | Term A, C- | |
| | Term Linker and | |
| | His8Tag | |
| 3 | CD40L-specific | IEVKDVTDTTALITWSDDFGEYVWCELTYGIKDVPGDRTTIDLWY |
| | Tn3 monomer | HHAHYSIGNLKPDTEYEVSLICRSGDMSSNPAKETFTT |
| | with affinity | |
| | mature variant | |
| | Clone 342- | |
| | Affinity mature | |
| | variant | |
| | (W/FG loop | |
| | variant RR -> | |
| | RS underlined) | |
| 4 | Human serum | DAHKSEVAHRFKDLGEENFKALVLIAFAQYLQQSPFEDHVKLVNE |
| | albumin variant | VTEFAKTCVADESAENCDKSLHTLFGDKLCTVATLRETYGEMADC |
| | | CAKQEPERNECFLQHKDDNPNLPRLVRPEVDVMCTAFHDNEETFL |
| | | KKYLYEIARRHPYFYAPELLFFAKRYKAAFTECCQAADKAACLLP |
| | | KLDELRDEGKASSAKQRLKCASLQKFGERAFKAWAVARLSQRFPK |
| | | AEFAEVSKLVTDLTKVHTECCHGDLLECADDRADLAKYICENQDS |
| | | ISSKLKECCEKPLLEKSHCIAEVENDEMPADLPSLAADFVESKDV |
| | | CKNYAEAKDVFLGMFLYEYARRHPDYSVVLLLRLAKTYETTLEKC |
| | | CAAADPHECYAKVFDEFKPLVEEPQNLIKQNCELFEQLGEYKFQN |
| | | ALLVRYTKKVPQVSTPTLVEVSRNLGKVGSKCCKHPEAKRMPCAE |
| | | DYLSVVLNQLCVLHEKTPVSDRVTKCCTESLVNRRPCFSALEVDE |
| | | TYVPKEFNAETFTFHADICTLSEKERQIKKQTALVELVKHKPKAT |
| | | KEQLKAVMDDFAAFVEKCCKADDKETCFAEEGKKLVAASQAALGL |
| 5 | beta strand “A” | IEV |
| | within a | |
| | CD40L-specific | |
| | monomer | |
| 6 | beta strand “B” | ALITW |
| | within a | |
| | CD40L-specific | |
| | monomer | |
| 7 | Linker | GGGGSGGGGS |
| 8 | Linker | GGGGSGGGGSGGGGS |
| 9 | Linker | GGGGGGGGGG |
| 10 | Linker | GGGGGGGGGGGGGGG |
| 11 | AB loop | KDVTDTT |
| 12 | BC loop | SDDFGEYVW |
| 13 | CD loop | KDVPGDR |
| 14 | DE loop | WYHHAH |
| 15 | EF loop | GNLKPDTE |
| 16 | FG loop | RSGDMSSNPA |
| 17 | beta strand “C” | CELTYGI |
| | within the | |
| | CD40L-specific | |
| | monomer | |
| 18 | beta strand “D” | TTIDL |
| | within a | |
| | CD40L-specific | |
| | monomer | |
| 19 | beta strand “E” | YSI |
| | within a | |
| | CD40L-specific | |
| | monomer | |
| 20 | beta strand “F” | YEVSLIC |
| | within a | |
| | CD40L-specific | |
| | monomer | |
| 21 | beta strand “G” | KETFTT |
| | within a | |
| | CD40L-specific | |
| | monomer | |
| 22 | CD40L-specific | SQIEVKDVTDTTALITWSDDFGEYVWCELTYGIKDVPGDRTTIDL |
| | Tn3 monomer | WYHHAHYSIGNLKPDTEYEVSLICRSGDMSSNPAKETFTT |
| | Clone 342- | |
| | variant | |
| | (W/FG loop | |
| | variant RR-> RS | |
| | underlined) | |
| 23 | beta strand | RLDAPSQIEV |
| | sequence “A” | |
| | within a | |
| | CD40L-specific | |
| | monomer | |
| 24 | beta strand “A” | SQIEV |
| | within a | |
| | CD40L-specific | |
| | monomer | |
| 25 | CD40L-specific | RLDAPSQIEVKDVTDTTALITWSDDFGEYVWCELTYGIKDVPGDR |
| | Tn3 monomer | TTIDLWYHHAHYSIGNLKPDTEYEVSLICRSGDMSSNPAKETFTT |

In aspects, compositions of the disclosure may comprise any of the amino acid sequences as described in Int'l Appl. Nos. PCT/US2012/059477 and PCT/US2019/052997, which are incorporated herein by reference in their entireties. In aspects, provided compositions may comprise the amino acid sequence as shown in SEQ ID NO: 1 (referred to herein as VIB4920). VIB4920 comprises a bivalent CD40L-specific Tn3 protein fused to a HSA protein.

If the Tn3 scaffold has amino acid sequence changes relative to those shown in SEQ ID NO: 1, the changes may be to the amino acid sequence of one or both of the CD40L-specific Tn3 monomers, so long as it does not adversely effect in vivo efficacy of the Tn3 scaffold, e.g., change in amino acid sequence such that one or both CD40L-specific Tn3 monomers have the amino acid sequence as shown in SEQ ID NO: 2, SEQ ID NO: 3, SEQ ID NO: 22, and SEQ ID NO: 25. In aspects, the first one or two N-terminal amino acid residues (SQ) may be absent and/or substituted with alternative amino acid residues.

In aspects, a Tn3 scaffold comprises at least one CD40L-specific monomer subunit bound to a heterologous moiety. In aspects, this heterologous moiety is selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a HSA FcRn binding portion, an antibody or fragment thereof, an albumin binding domain, an enzyme, a ligand, a receptor, a binding peptide, a non-FnIII scaffold, an epitope tag, a recombinant polypeptide polymer, a cytokine, and a combination of two or more of said moieties. In aspects, the heterologous moiety is the albumin and the albumin comprises human serum albumin. In aspects, the heterologous moiety is an antibody. In aspects, the antibody is selected from the group consisting of: an Fc domain of an antibody, an antibody fragment, and a single chain antibody.

In aspects, the heterologous moiety is an imaging agent; for example, a radionuclide or biotin. In aspects, the heterologous moiety is a drug; for example, a cytotoxic agent or a radioactive compound.

In aspects, the heterologous moiety comprises PEG. In aspects, the Tn3 scaffold comprises at least one CD40L-specific monomer subunit fused or conjugated directly or via a linker to PEG. In aspects, both CD40L-specific monomer subunits are fused, conjugated, or connected via a linker to PEG. In aspects, the Tn3 scaffold comprises at least one (e.g., two) CD40L-specific monomer subunit fused or conjugated directly or via a linker to PEG.

In aspects, the heterologous moiety comprises albumin. In aspects, the Tn3 scaffold comprises at least one CD40L-specific monomer subunit fused or conjugated directly or via a linker to an albumin. In aspects, this albumin is HSA. In aspects, this HSA is a variant HSA. In aspects, the amino acid sequence of the variant HSA is SEQ ID NO: 4. In aspects, the variant HSA has at least one improved property compared with a native HSA or a native HSA fragment. In aspects, the amino acid sequence of the variant HSA is SEQ ID NO: 4 or a sequence having at least about 80%, 85%, 90%, 95%, 96%,97%, 98%, 99%, or up to 100% identity to SEQ ID NO: 4. In aspects, the improved property is an altered plasma half-life compared with the plasma half-life of a native HSA or a native HSA fragment. In aspects, the altered plasma half-life is a longer plasma half-life compared with the plasma half-life of a native HSA or a native HSA fragment. In aspects, the altered plasma half-life is a shorter plasma half-life compared with the plasma half-life of a native HSA or a native HSA fragment.

### Dosing

In aspects, any of the compositions comprising a Tn3 scaffold of the disclosure can be administered in any form. In aspects, a Tn3 scaffold is administered intravenously, subcutaneously, orally, intramuscularly, intrathecally, sublingually, rectally, vaginally, cutaneously, systemically, topically, transdermally, or by way of inhalation. In aspects, a Tn3 scaffold is administered intravenously. In aspects, a Tn3 scaffold is administered by intravenous infusion.

A Tn3 scaffold of the disclosure can be administered at any dose. In aspects, a Tn3 scaffold is administered at a dose from about: 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, or about 5000 mg. Any of the aforementioned dosages may be effective dosages for a method comprising treatment, reduction, or elimination.

In aspects, a Tn3 scaffold is administered at a dose of between about: 800-5000 mg, 900-4900 mg, 1000-4800 mg, 1100-4700 mg, 1200-4600 mg, or 1300-4500 mg. In aspects, a Tn3 scaffold is administered at a dose selected from the group consisting of: 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, and 4500 mg. In aspects, a Tn3 scaffold is administered at a dose selected from the group consisting of: 1500 mg and 3000 mg. In aspects, a Tn3 scaffold is administered at a dose of about 1500 mg. In aspects, a Tn3 scaffold is administered at a dose of about 3000 mg.

### Dosing Frequency

In aspects, a Tn3 scaffold of the disclosure is administered on a schedule that provides optimal results. In aspects, a Tn3 scaffold is administered to a subject in need thereof about once a week, about twice a week, about every two weeks, about once a month, about every two months, about every 3 months, about every 12 weeks, about every fifteen weeks, about every sixteen weeks, about every four months, about every five months, about every six months, or semiannually.

Any number of administrations may be provided to a subject in need thereof. In aspects, a subject is administered an effective dose on about every Day 1, Day 2, Day 3, Day 4, Day 5, Day 6, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 1 year, 2 years, 3 years, 4 years, or 5 years, or up to the lifetime of a subject, post treatment initiation. In aspects, a subject receives an effective dose on Day 1, Day 15, Day 29, and Day 57 post treatment initiation. In aspects, a subject receives an effective dose on week 0, week 2, week 4, week 8, and week 12 post treatment initiation. In aspects, a subject receives 1500 mg-3000 mg of a Tn3 scaffold on Day 1, Day 15, Day 29, and Day 57 post treatment initiation. In aspects, a subject in need thereof is administered 1500 mg of a Tn3 scaffold on Day 1, Day 15, Day 29, and Day 57 post treatment initiation.

In aspects, a subject in need thereof is administered 1500 mg of a Tn3 scaffold on day 1 and day 57 post treatment initiation. In aspects, a subject in need thereof is administered 3000 mg of a Tn3 scaffold on Day 1, Day 15, Day 29, and Day 57 post treatment initiation. In aspects, a subject in need thereof is administered 3000 mg of a Tn3 scaffold on Day 1 and Day 57 post treatment initiation, and then every 6 months thereafter as needed. In aspects, a subject in need thereof is administered 1500 mg of a Tn3 scaffold at weeks 0, 2, 4, 8, and 12 post treatment initiation. In aspects, the administering comprises an induction dose, for example the 1500 mg of a Tn3 scaffold at weeks 0, 2, 4, 8, and 12 post treatment initiation. Any of the aforementioned administrations can deviate by about 0-5 days, 0-4 days, 0-3 days, 0-2 days, or by about 1 day.

In aspects, the induction dose comprises administering a Tn3 scaffold once about every 2 weeks for at least 2, 3, 4, 5, 6, 7, 8, 9, or up to about 10 doses. In aspects, the induction dose comprises administering a Tn3 scaffold once about every 2, 3, 4, 5, 6, 7, 8, 9, or 10 weeks for at least about 2, 3, 4, 5, 6, 7, 8, 9, or up to about 10 doses. In aspects, the induction dose comprises administering a Tn3 scaffold once about every 2 weeks for at least 3 doses.

In aspects, a Tn3 scaffold is administered at a dose of about 3000 mg every three months or quarterly.

In aspects, a quarterly administration of any of the provided compositions can be as efficacious as more frequent dosing, at the same dosage, with the added benefit of requiring less interventions of a subject being treated. Indeed, quarterly administration regimens can confer extended treatment as compared to otherwise comparable regimens requiring more frequent dosing as determined by any of the methodologies provided herein (e.g., adjusted mean change from baseline and the like).

In aspects, an administration is a quarterly administration of about 3000 mg of a Tn3 scaffold.

In aspects, a maintenance dose comprises administering a Tn3 scaffold once about every 2, 3, 4, 5, 6, 7, 8, 9, or 10 weeks for at least 2, 3, 4, 5, 6, 7, 8, 9, or up to about 10 doses. In aspects, the maintenance dose comprises administering a Tn3 scaffold once about every 4 weeks for at least 4 doses.

In aspects, a subject receives an effective dose of a Tn3 scaffold on Day 1, Day 15 (±1 day), Day 29 (±3 days), and Day 57 (±3 days) post treatment initiation. In aspects, a subject in need thereof is administered 1500 mg of a Tn3 scaffold on Day 1, Day 15 (±1 day), Day 29 (±3 days), and Day 57 (±3 days) post treatment initiation, and then every 6 months thereafter as needed. In aspects, a subject in need thereof is administered 1500 mg of a Tn3 scaffold on Day 1, Day 15 (±1 day), and Day 29 (±3 days) post treatment initiation. In aspects, a subject in need thereof is administered 1500 mg of a Tn3 scaffold on Day 1 and Day 57 (±3 days) post treatment initiation. In aspects, a subject in need thereof is administered 3000 mg of a Tn3 scaffold on Day 1, Day 15 (±1 day), Day 29 (±3 days), and Day 57 (±3 days) post treatment initiation, and then every 6 months thereafter as needed. In aspects, a subject in need thereof is administered 3000 mg of a Tn3 scaffold on Day 1, Day 15 (±1 day), and Day 29 (±3 days) post treatment initiation. In aspects, a subject in need thereof is administered 3000 mg of a Tn3 scaffold on Day 1 and Day 57 (±3 days) post treatment initiation, and then every 6 months thereafter as needed. In aspects, 3000 mg of a Tn3 scaffold is administered every 3 months.

In aspects, a Tn3 scaffold is administered at a dose of about 1500 mg once about every 2 weeks for at least 2 doses, and is administered about once a month thereafter. In aspects, a Tn3 scaffold is administered at a dose of about 1500 mg once about every 2 weeks for at least 3 doses, and is administered about once a month thereafter. In aspects, a Tn3 scaffold is administered at a dose of about 1500 once about every month, once about every two months, or once about every three months. In aspects, a Tn3 scaffold is administered at a dose of about 3000 mg once about every month, once about every two months, or once about every three months. In aspects, a Tn3 scaffold is administered for two or more doses.

In aspects, a subject in need thereof is administered an effective dose of a Tn3 scaffold once every 2-4 weeks. In aspects, a subject in need thereof is administered an effective dose of a Tn3 scaffold once every 2 weeks or 4 weeks. In aspects, a subject in need thereof is administered 1500 mg of a Tn3 scaffold once every 2 weeks for at least 3 doses, once every 4 weeks for at least 4 doses, once every 4 weeks for at least 5 doses, or a combination thereof. In aspects, a subject in need thereof is administered 3000 mg of a Tn3 Scaffold once every 2 weeks for at least 3 doses, once every 4 weeks for at least 4 doses, once every 4 weeks for at least 5 doses, or a combination thereof. In aspects, a subject in need thereof is administered an effective dose of a Tn3 scaffold as an induction dose and as a maintenance dose thereafter.

In aspects, a Tn3 scaffold of the disclosure comprises SEQ ID NO: 1. The Tn3 scaffold comprising SEQ ID NO: 1 can be administered to a subject in need thereof at an effective dose of about 1500 mg every 2 weeks for at least 3 doses, once every 4 weeks for at least 4 doses, once every 4 weeks for at least 5 doses, or a combination thereof. In aspects, the Tn3 scaffold comprising SEQ ID NO: 1 can be administered to a subject in need thereof at an effective dose of about 3000 mg once every 2 weeks for at least 3 doses, once every 4 weeks for at least 4 doses, once every 4 weeks for at least 5 doses, or a combination thereof. In aspects, a subject in need thereof is administered an effective dose of a Tn3 scaffold comprising SEQ ID NO: 1 as an induction dose and as a maintenance dose thereafter.

### Methods of Treatment

In aspects herein, methods are directed to treat, reduce, or eliminate RA. In aspects, a method comprises administering a Tn3 scaffold of the disclosure. In aspects, a Tn3 scaffold is used to treat RA. In aspects, a Tn3 scaffold is administered to a subject in need thereof to treat RA using any dosing schedules disclosed herein. In aspects, a Tn3 scaffold is administered at a dose of about 1500 mg once about every 2 weeks for at least 2 doses, and is administered about once a month thereafter. In aspects, a Tn3 scaffold is administered at a dose of about 1500 mg once about every 2 weeks for at least 3 doses, and is administered about once a month thereafter. In aspects, a Tn3 scaffold is administered at a dose of about 1500 once about every month, once about every two months, or once about every three months.

In aspects, a Tn3 scaffold is administered at a dose of about 3000 mg once about every month, once about every two months, or once about every three months. In aspects, a Tn3 scaffold is administered for two or more doses.

In aspects of the methods disclosed herein, a subject in need thereof has been administered one or more standard of care therapies for the treatment of RA prior to the administration of a Tn3 scaffold.

In aspects, a standard of care therapy comprises an anti-inflammatory agent, an anti-pain agent, and a regimen to reduce disability associated with RA. In aspects, a subject continues on one or more prior treatment therapies while being administered a Tn3 scaffold. In aspects, a standard of care therapy is administered as a second therapy. In aspects, any of the below referenced therapies may be combined in a therapeutic regimen for the treatment of RA. In aspects, a subject is treated with both a Tn3 scaffold and a TNFi. In aspects, a Tn3 scaffold and a TNFi can be administered within one day of each other but are not administered on the same day.

In aspects, any of the referenced therapies are combined as part of a treatment regimen or combination therapy. For example, anti-inflammatory medications, including nonsteroidal anti-inflammatory drugs and systemic and intraarticular corticosteroids, may be used in combination with DMARDs. In aspects, a Tn3 scaffold is administered sequentially with any therapy disclosed herein. In aspects, a Tn3 scaffold may generally be administered concurrently with any second therapy disclosed herein. In aspects, a subject in need thereof has been administered any standard of care therapy for the treatment of RA prior to the administration of a Tn3 scaffold.

In aspects, a Tn3 scaffold is administered to a subject in need thereof. In aspects, a subject in need thereof has moderate disease activity RA. In aspects, a subject in need thereof has high disease activity RA. Moderate or high disease activity is defined as a subject having a Simplified Disease Activity Index (SDAI) greater than or equal to 17 despite prior treatment for at least 12 weeks with a TNFi. In aspects, a TNFi may be selected from a group consisting of, but not limited to, infliximab, golimumab, certolizumab, etanercept, and adalimumab. Advantageously, methods and compositions disclosed herein are effective in achieving low disease activity, defined an SDAI less than or equal to 11, by 16 weeks of treatment. In aspects, methods and compositions disclosed herein are effective in achieving sustained remission, defined by an SDAI less than or equal to 3.3 as measured between 16 and 40 weeks of treatment. In aspects, a method allows tapering of a TNFi during the course of a Tn3 scaffold treatment. In aspects, a TNFi treatment may be halted.

### Anti-Inflammatory Agent

In aspects, a subject is administered an anti-inflammatory agent. In aspects, an anti-inflammatory agent comprises a nonsteroidal anti-inflammatory drug (NSAID). Exemplary NSAIDs are selected from the group consisting of: ibuprofen, naproxen, celecoxib, diclofenac, diflunisal, etodolac, indomethacin, ketoprofen, ketorolac, nabumetone, oxaprozin, piroxicam, salsalate, sulindac, tolmetin, and combinations thereof.

### Anti-Pain Agent

In aspects, a subject is administered an anti-pain agent. Anti-pain agents comprise NSAIDs, acetaminophen, corticosteroid, opioid (e.g., codeine, fentanyl, Vicodin, morphine, oxycodone, Percocet), anti-depressant, anti-histamine (e.g., diphenhydramine or cetirizine, and the like), anti-convulsant, lidocaine, and combinations thereof. In aspects, an anti-pain agent is an anti-histamine. In aspects, an anti-pain agent is acetaminophen.

Disease-modifying Antirheumatic Drug (DMARD). NSAIDs may be used as anti-pain agents as well as anti-inflammatory agents. Corticosteroids may be used as anti-pain agents as well as anti-inflammatory agents.

In aspects, a subject is administered a regimen to reduce disability associated with RA. In aspects, a subject is administered a disease-modifying antirheumatic drug (DMARD). In aspects, a DMARD is cDMARD. In aspects, a DMARD is bDMARD. Exemplary bDMARDs may be a TNFi; for example, selected from the group consisting of: etanercept, infliximab, adalimumab, certolizumab, and golimumab. In aspects, a TNFi is etanercept or adalimumab.

Suitable cDMARDs are selected from the group consisting of: methotrexate (MTX), hydroxychloroquine (Plaquenil), leflunomide (Arava), sulfasalazine (Azulfidine), and combinations thereof. In aspects, a cDMARD is selected from the group consisting of: methotrexate, sulfasalazine, leflunomide, and hydroxychloroquine. In aspects, a regimen to reduce disability comprises surgery. In aspects, a subject may undergo joint replacement surgery. Any body part may be subject of surgery including but not limited to hips, knees, shoulders, elbows, ankles, fingers, and combinations thereof. In aspects, joint fusion surgery is also performed. In aspects, a regimen comprises physical therapy. In aspects, a regimen comprises cognitive therapy. In aspects, a regimen comprises exercise.

### Corticosteroid

In aspects, a subject is administered a corticosteroid. In aspects, a corticosteroid comprises a glucocorticoid. Glucocorticoids (GCs) are steroid hormones used for the treatment of inflammation, autoimmune diseases, and/or cancer. To exert their broad physiological and therapeutic effects, GCs bind to the GC receptor (GR) which belongs to the nuclear receptor superfamily of transcription factors.

In aspects, one or more GCs are administered to a subject in need thereof. In aspects, one or more GCs is selected from the group consisting of: triamcinolone, methylprednisolone, budesonide, dexamethasone, triamcinolone, prednisone, hydrocortisone, dexamethasone, betamethasone, prednisolone, deflazacort, and combinations thereof. In aspects, a GC is prednisone. In aspects, a GC is methylprednisolone.

### Immune System Modifying Agent

In aspects, an immune system modifying agent comprises a cell-modifying agent, an interleukin, or a pathway modifier. In aspects, an immune system modifying agent is a cell-modifying agent. In aspects, a cell modifying agent modifies B cells or T cells. In aspects, a cell modifying agent modifies B cells and is selected from the group consisting of, but not limited to, rituximab, ofatumumab, obinutuzumab, and ibritumomab, and the like. In aspects, a cell modifying agent modifies B cells and comprises rituximab. In aspects, a cell modifying agent modifies T cells and is selected from the group consisting of, but not limited to, abatacept, belatacept, adalimumab, thymoglobulin, alemtuzumab, basiliximab, cyclosporine, sirolimus, tacrolimus, everolimus, and the like. In aspects, a cell modifying agent modifies T cells and comprises abatacept. In aspects, an immune system modifying agent binds an interleukin receptor selected from the group consisting of: IL-6R (tocilizumab), IL-2R, IL10R, IL-21R, and the like. In aspects, an immune system modifying agent is a pathway modifier that modifies a pathway such as Janus kinase (JAK; tofacitinib), and the like.

In aspects, a Tn3 scaffold of the disclosure is administered to a subject in need thereof in combination with a second therapy. In aspects, a second therapy comprises an anti-inflammatory agent, an anti-pain agent, a Disease-modifying Antirheumatic Drug (DMARD), a corticosteroid, or an immune system modifying agent. In aspects, a DMARD is a cDMARD or a bDMARD. In aspects, a cDMARD is selected from the group consisting of, but not limited to, hydroxychloroquine, leflunomide, sulfasalazine, tofacitinib, and combinations thereof. In aspects, a bDMARD is selected from the group consisting of, but not limited to, etanercept, infliximab, adalimumab, certolizumab, and golimumab.

In aspects, a Tn3 scaffold of the disclosure is administered to a subject in need thereof in combination with a TNF-α inhibitor. TNF-α plays a pro-inflammatory role in RA pathogenesis, with effects mainly on innate immunity. Among its many activities, it primes or directly mediates leukocyte activation, adhesion, and migration; endothelial activation and angiogenesis; chemokine expression; stromal cell activation; chondrocyte activation; and osteoclast differentiation in combination with receptor activator of NFκB ligand (RANKL). In contrast, IL-6 has prominent biological effects on both innate and adaptive immunity in RA. IL-6 promotes CD4+ T cells differentiation into T helper 17 (Th17) cells in combination with transforming growth factor-β (TGF-β) and inhibits TGF-β-driven T regulatory (Treg) cell differentiation, resulting in up-regulation of Th17/Treg balance and disruption of tolerance mechanisms. IL-6 also promotes T follicular helper cell differentiation and induces activated B cells differentiation of into antibody-secreting cells. Inhibition of the CD40-CD40L pathway by way of a Tn3 scaffold would be expected to specifically suppress the adaptive immune response. Combining a Tn3 scaffold with a TNF-α inhibitor provides an opportunity to determine if RA is driven by varied contributions of antigen-dependent (adaptive) and antigen-independent (innate) immune mechanisms.

Accordingly, combining TNF-α inhibition with blockade of the CD40-CD40L pathway by disclosed Tn3 scaffolds disrupts the pathologic innate and adaptive immune responses in a manner that restores normal tolerance mechanisms and promotes sustained disease control. In aspects, treatment with a TNFi in combination with a Tn3 scaffold will be more effective in controlling disease activity than maintaining a TNFi alone or replacing a partially effective TNFi with a Tn3 scaffold. In aspects, treatment with a TNFi in combination with a Tn3 scaffold is effective in reducing disease or a symptom in a subject in need thereof by at least about 1-fold, 10-fold, 50-fold, 100-fold, 200-fold, 500-fold, or up to about 1000-fold as compared to an otherwise comparable subject not administered a Tn3 scaffold. In aspects, treatment with a TNFi in combination with a Tn3 scaffold is effective in stabilizing disease than maintaining a TNFi alone or replacing a partially effective TNFi with a Tn3 scaffold.

In aspects, one or more standard of care therapies are administered for about 1, about 2, about 3, about 4, about 5, about 6, about 7, about 8, about 9, about 10, about 11, about 12, about 13, about 14, about 15, about 20, about 25, about 30, about 35, about 40, about 52, about 52 weeks or more prior to the administration of a Tn3 scaffold.

A dose and dosing regimen of a Tn3 scaffold as disclosed herein may be such that any therapeutic effect achieved from administration of a Tn3 scaffold to treat any autoimmune/inflammatory disease or disorder, may be considered to be “long-lasting.” A “long-lasting” effect of a Tn3 scaffold in a treatment of an autoimmune/inflammatory disease or disorder is one in which a therapeutic effect achieved by a Tn3 scaffold is maintained (although the Tn3 scaffold is no longer administered) over at least 4 weeks, at least 6 weeks, at least 8 weeks, at least 10 weeks, at least 12 weeks, at least 16 weeks, at least 20 weeks, or at least 24 weeks following administration of the last dose of a course of a Tn3 scaffold. In aspects, less frequent dosing of any of the compositions provided herein may be advantageous. Exemplary advantages of less frequent dosing include but are not limited to reduced frequency of side effects associated with an administered composition, reduced treatment-associated toxicity, increased quality of life for treated subjects, and the like. In aspects, a single administration of about 3000 mg of a Tn3 scaffold can be as equally efficacious or more efficacious than two or more administrations of the same composition as determined by the change from baseline in DAS28-CRP of a subject administered a Tn3 scaffold. In aspects, about 3000 mg once every 8 weeks (Q8W; administered twice) is about as efficacious as 1500 mg once every 4 weeks (Q4W). In aspects, provided are methods comprising a longer dosing interval as compared to otherwise comparable methods lacking administration of a Tn3 scaffold of the disclosure.

### Assessment of Treatment Efficacy

In aspects, a subject is assessed. An assessment can occur at any point before, during, or after administration with a Tn3 scaffold. In aspects, an assessment is performed before administration. In aspects, an assessment is performed during administration. In aspects, an assessment is performed post administration.

Any of the below-referenced assessments can occur at any time. In aspects, a subject is assessed by the minute, hourly, daily, weekly, monthly, quarterly, or yearly. In aspects, an assessment is completed twice daily, biweekly, bimonthly, or semiannually. In aspects, an assessment is performed from day −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308 or up to about day 309 post treatment. Timing of assessments are further described below.

In aspects, a treatment regimen that comprises quarterly administrations of a composition that comprises a Tn3 scaffold is as efficacious as an otherwise comparable composition, at the same dosage, lacking the quarterly administration. In aspects, a treatment regimen that comprises administrations every 8 weeks of a composition that comprises a Tn3 scaffold is as efficacious as an otherwise comparable composition, at the same dosage, lacking the administration every 8 weeks. In aspects, a treatment regimen that comprises administrations every 12 weeks of a composition that comprises a Tn3 scaffold is as efficacious as an otherwise comparable composition, at the same dosage, lacking the administration every 12 weeks. In aspects, quarterly administration of a composition that comprises a Tn3 scaffold confers sustained treatment efficacy following a prior administration of a Tn3 scaffold as determined by an assessment provided herein. In aspects, treatment efficacy is determined by any of the following assessments. In aspects, sustained treatment efficacy is maintained for a period of about 5-10 days, 10-30 days, 15-40 days, 1 month-3 months, 1 month-2 months, 2 months-3 months, 3 months-5 months, 1 month-5 months, 1 year, 2 years, or up to about 5 years following a prior administration of a Tn3 scaffold. In aspects, sustained treatment efficacy is maintained for a period of about 5,7,9,11,13,15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99,101,103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, or up to about 400 days post treatment. The sustained treatment efficacy may be characterized by an adjusted mean score of −0.5, −1.0 from baseline as measured at the period post treatment.

In aspects, a subject undergoing quarterly administrations of a Tn3 scaffold has comparable or greater treatment efficacy as compared to an otherwise comparable subject lacking the quarterly administration or undergoing more frequent administration of the same Tn3 scaffold. In aspects, a subject undergoing administrations of a Tn3 scaffold every 8 or 12 weeks has comparable or greater treatment efficacy as compared to an otherwise comparable subject lacking the every 8 or 12 week administration or undergoing more frequent administration of the same Tn3 scaffold.

In aspects, a treatment of RA may be characterized by a reduction of at least about 10%, about 20%, about 30%, about 40%, about 50% or more of clinical symptoms of the disease or disorder, or by a reduction in inflammation, or by a reduction in biomarkers of the disease or disorder, relative to their levels prior to the treatment with a Tn3 scaffold. A reduction of any of these symptoms, or inflammation, or biomarkers, may be a reduction in the symptoms, or inflammation or biomarkers of at least about 25%, about 30%, about 40%, about 50%, about 60%, about 70%, about 75%, or more relative to their levels prior to the initiation of treatment with a Tn3 scaffold. A reduction may be such that the autoimmune disease or disorder is characterized as being in remission. If a Tn3 scaffold is used in a method of reducing inflammation in an inflammatory disease or disorder, an inflammatory disease or disorder may comprise RA.

In aspects, a sample of a subject is analyzed. In aspects, a sample of a subject is analyzed pre-treatment. In aspects, a sample of a subject is analyzed post-treatment. In aspects, a sample of a subject is analyzed pre-treatment and post-treatment. In aspects, a sample of a subject is analyzed pre-treatment for soluble factors. In aspects, a sample of a subject is analyzed post-treatment for soluble factors. In aspects, a sample of a subject is analyzed pre-treatment and post-treatment for soluble factors. In aspects, a sample of a subject is analyzed pre-treatment and post-treatment for soluble factors. The aforementioned soluble factors may include but are not limited to: levels of antibodies or autoantibodies, such as rheumatoid factor (RF) and anti-cyclic citrullinated peptide antibody (ACPA); levels of sCD40L; levels of anti-drug antibodies targeting adalimumab, etanercept, and/or a Tn3 scaffold; levels of a Tn3 scaffold; levels of adalimumab or etanercept, as applicable; levels of CXCL13, sICAM, and/or any other relevant soluble mediators (e.g., IL-21, IL-10, IL-2, IL-17A, IFNγ, and the like.).

Levels of soluble immune parameters may be compared between a baseline value and time points following treatment with a composition disclosed herein. Comparisons may be made between treatment groups to evaluate an effect of the therapeutic intervention on changes in circulating levels of soluble immune parameters. In addition, a relationship between baseline sICAM and CXCL13 levels and treatment response can be determined. An impact of coadministration of a TNFi and a Tn3 scaffold on the drug levels of a Tn3 scaffold and adalimumab or etanercept may be assessed using parameters such as AUC, Cₘₐₓ, and t_{1/2} (half-life). Finally, levels of soluble immune parameters may also be evaluated for associations with frequency, phenotype, and/or functional profile of circulating cells, such as T cell, B cell and myeloid populations.

In aspects, a treatment of RA may be a reduction of one or more of RF autoantibodies, anti-citrullinated peptide antibodies, Vectra DA biomarker score (Vectra DA biomarker score being a composite score of expression levels of interleukin-6, tumor necrosis factor receptor type I, vascular cell adhesion molecule 1, epidermal growth factor, vascular endothelial growth factor A, YKL-40, matrix metalloproteinase 1, MMP-3, serum reactive C protein (CRP), serum amyloid A, leptin, and resistin), plasma cell (PC) signature, CRP, DAS28-CRP, or clinical disease activity index (CDAI), or may be a reduction in number of tender joints, intensity of joint tenderness, number of swollen joints, or intensity of joint swelling, or any combinations thereof. In aspects, a treatment may be achievement of American College of Rheumatology (ACR) response criteria ACR20, ACR50, or ACR70.

### Immunogenicity

In aspects, an assessment comprises determining a level of immunogenicity, if any, of a Tn3 scaffold of the disclosure. Immunogenicity comprises determining the presence of anti-drug antibodies (ADA) to a Tn3 scaffold. Immunogenicity may comprise determining the presence of anti-drug antibodies (ADA) to a TNFi. The presence of ADA can be evaluated using a plasma sample from a subject administered a Tn3 scaffold. In aspects, ADA are not detected post administration of a Tn3 scaffold and/or a TNFi. In aspects, ADA levels are reduced as compared to an otherwise comparable method wherein the subject of the otherwise comparable method is not administered a Tn3 scaffold; for example, the reduction may be about: 10%, 15%, 20%, 25%, 50%, 60%, 70%, 80%, 90%, 95%, 96%, 97%, 98%, 99%, or up to 100% as compared to ADA levels in an otherwise comparable method wherein the subject of the otherwise comparable method is not administered a Tn3 scaffold.

In aspects, determining ADA levels to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, determining ADA levels to a Tn3 scaffold can be assessed on about Day 1, Day 15±1, Day 29±3, Day 85±3, Day 169 f 5, Day 253±7, Day 309±7 post treatment initiation.

### Autoantibodies

In aspects, a method for treating, reducing, or eliminating RA may be characterized by an assessment comprises determining a level of autoantibodies in a subject. Autoantibodies comprise those that react with self-antigens. Exemplary autoantibodies comprise RF isotypes such as IgG, IgA, IgM, and combinations thereof. In aspects, an autoantibody can also be an anti-carbamylated protein antibody (anti-CarP) or antibodies against citrullinated protein and peptides (ACPA), or combinations thereof.

Exemplary methods of evaluating levels of autoantibodies may comprise EliA immunoassay, microarray, ELISA, or combinations thereof. In aspects, autoantibodies are not detected post administration of a Tn3 scaffold. In aspects, autoantibodies are not detected post administration of a Tn3 scaffold and a TNFi. In aspects, autoantibodies are detected at levels of at most about: 1-fold, 2-fold, 3-fold, 4-fold, 5-fold, 10-fold, 15-fold, 20-fold, 25-fold, 30-fold, 35-fold, 40-fold, 45-fold, or 50-fold as compared to an otherwise comparable method wherein the subject of the otherwise comparable method is not administered a Tn3 scaffold. In aspects, treatment comprises reducing autoantibodies in a subject by about: 20%, 30%, 40%, 45%, 50%, 60%, 75%, 80%, 90%, 95%, 96%, 97%, 98%, 99%, or up to 100% as compared to of the levels of autoantibodies in an otherwise comparable method wherein the subject of the otherwise comparable method is not administered a Tn3 scaffold.

In aspects, determining autoantibodies levels to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, determining autoantibodies levels to a Tn3 scaffold can be assessed on about Day 1, Day 15±1, Day 29±3, Day 57±3, Day 85±3, Day 113±5, Day 141±5, Day 169±5, Day 197±±, Day 225±±, Day 253±7, Day 281±7, Day 309±7 post treatment initiation.

### Inflammation

In aspects, an assessment comprises determining a level of markers of inflammation post treatment with a Tn3 scaffold. Exemplary markers of inflammation include but are not limited to: IgM, IgG, IgA, C reactive protein (CRP), and combinations thereof. In aspects, suitable assays to assess a level of inflammation comprise: ELISA, hs-CRP test, CRP test, Luminex, and combinations thereof. In aspects, treated, reduced, or eliminated inflammation is detected in a subject administered a Tn3 scaffold. In aspects, a change as compared to a baseline is determined. In aspects, inflammation is reduced by at least about 1-fold, 2-fold, 3-fold, 4-fold, 5-fold, 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or up to about 300-fold post administration as compared to an otherwise comparable method wherein the subject of the otherwise comparable method is not administered a Tn3 scaffold. In aspects, treatment comprises reducing markers of inflammation in a subject by about: 20%, 30%, 40%, 45%, 50%, 60%, 75%, 80%, 90%, 95%, 96%, 97%, 98%, 99%, or up to 100% as compared to the levels of markers of inflammation in an otherwise comparable method wherein the subject of the otherwise comparable method is not administered a Tn3 scaffold.

In aspects, determining levels of markers of inflammation levels to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation.

In aspects, determining levels of markers of inflammation to a Tn3 scaffold can be assessed on about Day 1, Day 15 1, Day 29 3, Day 57 3, Day 85 3, Day 113 5, Day 141 5, Day 169±, Day 197±7, Day 225±7, Day 253±7, Day 281±7, Day 309±7 post treatment initiation.

### Response Duration (Duration of Clinical Response)

In aspects, an assessment comprises determining duration of a response to a Tn3 scaffold by way of quantifying time to initiation of a rescue therapy. In aspects, rescue therapy comprises one or more of: administration of a new or intensified immunosuppressive, cDMARD, or bDMARD treatment for RA (e.g., initiation of or increase in dose of any cDMARD; initiation of a bDMARD therapy or JAK inhibitor therapy), increase in baseline corticosteroid dose, intraarticular steroid injection (e.g., methylprednisolone (or an equivalent)), or an intraarticular steroid injection of any dose. In aspects, an intraarticular steroid injection of any dose is administered at least once. In aspects, administration of a Tn3 scaffold is effective in treating, reducing, or eliminating initiation of a rescue therapy in a treated subject as compared to an otherwise comparable method wherein the subject of the otherwise comparable method is not administered a Tn3 scaffold. In aspects, administration of a Tn3 scaffold is effective at extending time to initiation of rescue therapy by at least about: 1 day, 6 days, 11 days, 16 days, 21 days, 26 days, 30 days, 2 months, 3 months, 4 months, 5 months, 6 months, 9 months, 11 months, 1 year, 2 years, or up to about 5 years post administration.

### Cytokine and Immune Cell Level

In aspects, an assessment can comprise determining a level of a cytokine or an immune cell. In aspects, an immune cell comprises a leukocyte. In aspects, an immune cell comprises a lymphocyte. In aspects, an immune cell comprises a T cell or a B cell. In aspects, a level of a T cell is determined. Exemplary T cells comprise any of CD3, CD4, or CD8 positive cells. In aspects, levels of CD4+ T cells are determined including but not limited to: Th1, Th17, T reg, T helper, Th22, Th2, and combinations thereof. In aspects, levels of CD8 T cells are determined including but not limited to Tc1, Tc2, Tc9, Tc17, Tc22, and combinations thereof. In aspects, a cytokine level is determined. Exemplary cytokines include but are not limited to: CXCL13, free sCD40L, IL-6, interleukin-1 (IL-1), IL-12, and IL-18, tumor necrosis factor alpha (TNF-α), interferon gamma (IFNγ), interferon alpha (IFNα) granulocyte-macrophage colony stimulating factor (GM-CSF), and combinations thereof. In aspects, a change in level of any of the above referenced cytokines or immune cells is quantified and compared to a baseline level. In aspects, cytokine and/or immune cell levels are reduced by at least about 1-fold, 2-fold, 3-fold, 4-fold, 5-fold, 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or up to about 300-fold post administration as compared to an otherwise comparable method wherein the subject of the otherwise comparable method is not administered a Tn3 scaffold. In aspects, cytokine and/or immune cell levels are reduced by about: 10%, 15%, 20%, 25%, 50%, 60%, 70%, 80%, 90%, 95%, 96%, 97%, 98%, 99%, or up to 100% as compared to an otherwise comparable method wherein the subject of the otherwise comparable method is not administered a Tn3 scaffold. Suitable methods to determine immune cell levels or cytokine levels may comprise flow cytometry, ELISA, Luminex, and combinations thereof by way of collecting blood and/or serum from a subject.

In aspects, determining levels of a cytokine and/or an immune cell to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation.

In aspects, determining levels of a cytokine and/or an immune cell to a Tn3 scaffold can be assessed on about Day 1, Day 15 1, Day 29±3, Day 57±3, Day 85±3, Day 113±5, Day 141±5, Day 169±5, Day 197±7, Day 225±7, Day 253±7, Day 281±7, Day 309±7 post treatment initiation.

### RNA and DNA Analysis

In aspects, an assessment comprises quantifying expression levels of genes associated with disease activity by way of RNA and/or DNA analysis. In aspects, RNA testing may be performed to measure expression levels of genes associated with disease activity, specific cell types including plasma cells, T follicular helper cells, and pathways implicated in the pathogenesis of RA, including the CD40L/CD40 pathway. RNA may be isolated at baseline from blood to test for changes in expression levels of the aforementioned genes associated with disease activity. In aspects, transcriptome profiling is assessed by methods including, but not limited to, qPCR, RNAseq, and exome sequencing. In aspects, DNA testing may be performed. DNA may be isolated at baseline from blood to test for CD40/CD40L polymorphisms or polymorphisms in other genes relevant to the mechanism of action of a Tn3 scaffold.

In aspects, DNA epigenetics analysis may be performed. In aspects, DNA epigenetics analysis includes assessing changes in DNA methylation in immune-related genes. In aspects, changes are determined as compared to a baseline level. In aspects, epigenome profiling is assessed by methods including, but not limited to, ATACseq and DNA methylation sequencing.

In aspects, DNA may be collected for genotyping or sequencing of relevant disease- or immune-associated genes, such as HLA Class I/II alleles, genes with reported associations to RA, or genes related to the CD40L-CD40 pathway to investigate correlations with disease activity and therapeutic response. Similarly, whole blood may be used to examine epigenetic status of relevant disease- or immune-associated genes and to investigate relationships between epigenetics and disease activity or therapeutic response.

In aspects, whole blood can be collected and may be used to evaluate gene expression profiles before, during, and after treatment with any of the compositions provided herein. Gene expression of molecules found to be modulated by treatment in blood leukocytes may be analyzed in whole blood using quantitative methods. Samples may be used to examine gene expression signatures of various cell types and their changes over time, as well as to explore whether the circulating fibroblast and activated B cell gene signatures detected shortly before a disease flare as assessed by RAPID3.

In aspects, expression levels of genes associated with disease activity by way of RNA and/or DNA analysis may be reduced by at least about 1-fold, 2-fold, 3-fold, 4-fold, 5-fold, 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or up to about 300-fold post administration as compared to an otherwise comparable method wherein the subject of the otherwise comparable method is not administered a Tn3 scaffold. In aspects, expression levels of genes associated with disease activity by way of RNA and/or DNA analysis may be reduced by at least about 10%, 15%, 20%, 25%, 50%, 60%, 70%, 80%, 90%, 95%, 96%, 97%, 98%, 99%, or up to 100% as compared to an otherwise comparable method wherein the subject of the otherwise comparable method is not administered a Tn3 scaffold.

In aspects, determining expression levels of genes associated with disease activity by way of RNA and/or DNA analysis can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, determining expression levels of genes associated with disease activity by way of RNA and/or DNA analysis can be assessed on about Day 1, Day 15±1, Day 29 3, Day 57 3, Day 85 3, Day 113 5, Day 141 5, Day 169±, Day 253 7, and Day 309 f 7 post treatment initiation.

### Pharmacokinetic Assessment

In aspects, a method provided herein can comprise determining a concentration of a Tn3 scaffold in a subject in need thereof post administration. In aspects, a method comprises a pharmacokinetic assessment. In aspects, a sample is a blood sample or a plasma sample, or a combination of both. In aspects, a suitable assay to measure pharmacokinetics may comprise electrochemiluminescence (ECL) assay, a bead-based assay, a cell-based assay, and combinations thereof. In aspects, a sample may comprise plasma and the plasma is assessed for a Tn3 scaffold concentration by measuring: maximum observed concentration (C.), area under the concentration-time curve (AUC), CL, and terminal elimination half-life (tim).

In aspects, a pharmacokinetic assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a pharmacokinetic assessment to a Tn3 scaffold can be assessed on about Day 1, Day 15±1, Day 29±3, Day 57±3, Day 85±3, Day 113±5, Day 141±5, Day 169±5, Day 197±7, and Day 225±7 post treatment initiation.

### Pharmacodynamic Assessment

In aspects, a method comprises a pharmacodynamic assessment. In aspects, an assessment can occur over a period of time. In aspects, a method provided herein can comprise determining an amount of reduction or elimination of sCD40L by a Tn3 scaffold in a subject in need thereof post administration.

In aspects, a reduction of sCD40L may be detected as compared to an otherwise comparable method wherein the subject of the otherwise comparable method is not administered a Tn3 scaffold. In aspects, elimination of sCD40L may be detected as compared to an otherwise comparable method wherein the subject of the otherwise comparable method is not administered a Tn3 scaffold as compared to an otherwise comparable method lacking the administering of the Tn3 scaffold. In aspects, reduction of sCD40L comprises at least about or at most about: 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 105-fold, 110-fold, 115-fold, 120-fold, 125-fold, 130-fold, 135-fold, 140-fold, 145-fold, 150-fold, or up to about 200-fold reduction as compared to an otherwise comparable method lacking the administering.

The disclosure provides for a Tn3 scaffold containing compositions that efficiently reduce or deplete sCD40L in a subject. Because s Tn3 scaffold binds to and depletes sCD40L, the reduction or elimination can be used as a measure of treatment efficacy. In aspects, treatment efficacy of a Tn3 scaffold on sCD40L can be assessed over time using a suitable immunoassay. In aspects, effects of a Tn3 scaffold are assessed over time using a qualified immunoassay. In aspects, effects of a Tn3 scaffold with and without a TNFi are assessed over time using a qualified immunoassay. In aspects, total sCD40L is a measure of target engagement. Suitable immunoassays may comprise flow cytometry, histology, immunohistochemistry, blood analysis, microscopy, PCR, ELISA, and combinations thereof.

In aspects, a Tn3 scaffold may achieve at least about 10%, 15%, 20%, 25%, 50%, 60%, 70%, 80%, 90%, 95%, 96%, 97%, 98%, 99%, or up to 100% reduction in sCD40L levels as compared to an otherwise comparable method wherein the subject of the otherwise comparable method is not administered a Tn3 scaffold. Reduction or elimination of sCD40L may persist for extended periods of time. In aspects, sCD40L depletion may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, sCD40L depletion may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In aspects, sCD40L depletion may persist for at least 1 month, at least 2 months, at least 3 months, at least 4 months, at least 5 months, at least 6 months, at least 7 months, at least 8 months, at least 9 months, at least 10 months, at least 11 months or at least 12 months. In aspects, sCD40L depletion may be improved when a Tn3 scaffold is combined with a TNFi.

In aspects, an assessment comprises determining composite disease score. Composite disease scores are selected from the group consisting of, but not limited to: DAS28-CRP, CDAI, SDAI, 28-Joint Count (TJC and SJC), MDGA, PGA, CRP, and combinations thereof. In aspects, a change from baseline may be determined in any of the aforementioned disease scores. In aspects, a disease score may be reduced post administration of a composition provided herein. In aspects, a disease may be treated post administration of a composition provided herein. In aspects, a disease may be reduced post administration of a composition provided herein. In aspects, a disease may go into remission post administration of a composition provided herein. In aspects, a disease may be eliminated post administration of a composition provided herein. In aspects, low disease activity comprises a DAS28-CRP≤3.2 and an improvement of DAS28-CRP score>0.6 from baseline, and/or a proportion of subjects with remission defined as CDAI≤2.8 or SDAI≤3.3.

In aspects, a pharmacodynamic assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a pharmacodynamic assessment to a Tn3 scaffold can be assessed on about Day 1, Day 15±1, Day 29±3, Day 57±3, Day 85±3, Day 113±5, Day 1415, Day 169±5, Day 197±7, Day 225±7, Day 253±7, Day 281±7, and Day 309±7 post treatment initiation.

### Disease Activity Score in 28 Joints Using C-reactive Protein (DAS28-CRP)

In aspects, an assessment comprises DAS28-CRP. DAS28-CRP is a composite score that includes an assessment of 28 specified joints for tenderness and swelling (TJC and SJC), the PGA, and CRP levels (mg/L) (Table 5). Calculation of a DAS28-CRP score may be as follows: DAS28-CRP=0.56×(TJC28)+0.28×(SJC28)+0.014×PGA+0.36×ln(CRP+1)+0.96 (Formula 1), where TJC28 is a TJC using 28 joints, SJC28 is a SJC using 28 joints, and PGA is a patient global assessment on a scale 0-100 mm. The range of a DAS28-CRP score is 0.96-9.31.

In aspects, remission by DAS28-CRP score may be DAS28-CRP≤2.6 (Anderson J, Caplan L, Yazdany J, Robbins ML, Neogi T, Michaud K, et al. Rheumatoid arthritis disease activity measures: American College of Rheumatology recommendations for use in clinical practice. Arthritis Care Res. 2012; 64(5):640-7). Low disease activity may be defined as DAS28 CRP≤3.2, and an improvement of DAS28 CRP score>0.6 may define a responder (Wells G, Becker J C, Teng J, Dougados M, Schiff M, Smolen J, et al. Validation of the 28-joint Disease Activity Score (DAS28) and European League Against Rheumatism response criteria based on C-reactive protein (CRP) against disease progression in subjects with rheumatoid arthritis, and comparison with the DAS28 based on erythrocyte sedimentation rate. Ann Rheum Dis. 2009; 68(6):954-60). In aspects, a change from baseline is determined

In aspects, a DAS28-CRP assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a DAS28-CRP assessment to a Tn3 scaffold can be assessed on about Day 1, Day 15±1, Day 29±3, Day 57±3, Day 85±3, Day 113±5, Day 1415, Day 169±5, Day 197±7, Day 225±7, Day 253±7, Day 281±7, and Day 309±7 post treatment initiation.

### Clinical Disease Activity Index (CDAI)

In aspects, an assessment comprises CDAI. CDAI may be a clinical composite score that includes but is not limited to: 28-joint count, MDGA, PGA, and combinations thereof. Remission comprises an CDAI≤about 2.8. In aspects, remission comprises an CDAI of about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4. In aspects, remission comprises a CDAI of at most or at least about 3.3. In aspects, remission comprises a CDAI from about: 1-2.8, 1.5-3, 2-3, 2-2.8, 1.8-2.8, or 2.2-3. In aspects, a change from baseline is determined.

In aspects, CDAI assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, CDAI assessment to a Tn3 scaffold can be assessed on about Day 1, Day 15 1, Day 29 3, Day 57 3, Day 85 3, Day 113 5, Day 141 f 5, Day 169±5, Day 253±7, and Day 309±7 post treatment initiation.

### Simplified Disease Activity Index (SDAI)

In aspects, an assessment comprises SDAI. SDAI may be a composite score that includes, but is not limited to: DAS28-CRP, 28-joint count, PGA, and CRP, and combinations thereof. In aspects, remission comprises an SDAI of about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4. In aspects, remission comprises SDAI≤about 3.3. In aspects, remission comprises an SDAI of at most or at least about 3.3. In aspects, remission comprises an SDAI from about: 2-3.3, 3-3.3, 3-3.4, 3.1-3.4, 3.1-3.3, 3-4, 3.1-3.4, or 3.2-3.4. Change may also be measured versus baseline.

In aspects, SDAI assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, SDAI assessment to a Tn3 scaffold can be assessed on about Day 1, Day 15 1, Day 29 3, Day 57±3, Day 85±3, Day 113±5, Day 141 f 5, Day 169±5, Day 253±7, and Day 309±7 post treatment initiation. 28-Joint Count

In aspects, an assessment comprises 28-Joint Count (TJC and SJC). TJC and SJC assess 28 specified joints for tenderness and swelling. In aspects, 28 specified joints comprise, but are not limited to: the 8 proximal interphalangeal joints of the fingers, the 2 interphalangeal joints of the thumbs, the 10 metacarpophalangeal joints, and the wrists, elbows, shoulders, and knees. Change may be measured versus baseline.

In aspects, a TJC and SJC assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a TJC and SJC assessment to a Tn3 scaffold can be assessed on about Day 1, Day 15±1, Day 29±3, Day 57±3, Day 85±3, Day 113±5, Day 141±5, Day 169±5, Day 197±7, Day 225±7, Day 253±7, Day 281±7, and Day 309±7 post treatment initiation.

### Physician Global Assessment of Disease Activity (MDGA)

In aspects, an assessment comprises MDGA. In aspects, MDGA may comprise determining a subject's level of disease activity due to RA on a scale of 0 (no disease) activity to 100 (maximum disease activity) on a 100 mm visual analog scale (VAS) (Rohekar and Pope, 2009). In aspects, MDGA may be scored from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and up to about 100.

In aspects, MDGA is from about 1-10, about 10-30, about 20-40, about 30-50, about 40-60, about 50-70, about 60-80, about 70-90, or about 80-100. In aspects, a change from baseline in MDGA is determined.

In aspects, a MDGA assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a MDGA assessment to a Tn3 scaffold can be assessed on about Day 1, Day 15±1, Day 29±3, Day 57±3, Day 85±3, Day 113±5, Day 141±5, Day 169±5, Day 197±7, Day 225±7, Day 253±7, Day 281±7, and Day 309±7 post treatment initiation.

### Patient Global Assessment of Disease Activity (PGA)

In aspects, an assessment comprises PGA. PGA is a measure of a subject's general health (Rohekar G, Pope J. Test-retest reliability of patient global assessment and physician global assessment in rheumatoid arthritis. J Rheumatol. 2009; 36(10):2178-82). In aspects, a subject can be asked to rate their current quality of life by making a mark on a 100 mm VAS in response to these exemplary instructions: “Considering all the ways that your rheumatoid arthritis affects you, please rate how well you are doing on a scale of 0 (very well) to 100 (very poorly).” In aspects, PGA is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or up to about 100. In aspects, GA is from at least about 1-10, about 10-30, about 20-40, about 30-50, about 40-60, about 50-70, about 60-80, about 70-90, or about 80-100. In aspects, a change from baseline in PGA is determined.

In aspects, a PGA assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a PGA assessment to a Tn3 scaffold can be assessed on about Day 1, Day 15±1, Day 29±3, Day 57±3, Day 85±3, Day 113±5, Day 141 f 5, Day 169±5, Day 197±7, Day 225±7, Day 253±7, Day 281±7, and Day 309±7 post treatment initiation.

### FACIT-Fatigue Scale and HAQ

In aspects, an assessment comprises a FACIT-Fatigue scale and/or a HAQ. A FACIT-Fatigue Scale is a 13-item subject-completed questionnaire used to assess the impact of fatigue. The FACIT-Fatigue scale recall period is about 7 days. Responses to a FACIT-Fatigue questionnaire range from 0 (Not at all) to 4 (Very much). In aspects, a FACIT-fatigue score is at least about 0, about 1, about 2, about 3, or about 4.

In aspects, a HAQ is a subject-completed questionnaire used to assess a subject's ability to perform activities of daily living and a subject's pain due to illness. The HAQ recall period is about 7 days. HAQ questions may be selected from the following exemplary groups: Dressing & Grooming, Arising, Eating, Walking, Hygiene, Reach, Grip, and Activities, or combinations thereof. Four response categories are available for each question, from 0 (Without any difficulty) through 4 (Unable to do).

Subjects are asked about their use of aids and devices and if they need help from another person to complete activities. A HAQ also includes a VAS pain score asking, “How much pain have you had because of your illness in the past week”, with a line for the subject to mark between 0 (No pain) and 100 (Severe pain). In aspects, a baseline level of any one of the aforementioned assays is reduced by at least about 1, about 2, about 3, or about 4 points post treatment with a composition provided herein. In aspects, a subject's score is reduced as compared to an otherwise comparable method wherein the subject of the otherwise comparable method is not administered a Tn3 scaffold.

In aspects, a FACIT-Fatigue scale and/or HAQ assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a FACIT-Fatigue scale and/or HAQ assessment to a Tn3 scaffold can be assessed on about Day 1, Day 1, Day 29 3, Day 57 3, Day 85 3, Day 113 5, Day 141 5, Day 169±, Day 197 7, Day 225±7, Day 253±7, Day 281±7, and Day 309±7 post treatment initiation.

### PROMKS-29 Profile

In aspects, an assessment comprises a PROMIS-29 profile. A PROMIS-29 adult profile is a brief generic health measure comprising 29-items from the PROMIS domains of anxiety, depression, fatigue, pain (intensity and interference), physical function, sleep disturbance, satisfaction with participation in social roles (social participation), or combinations thereof. In aspects, a treated subject has an improved score on a PROMIS-29 as compared to an otherwise comparable method wherein the subject of the otherwise comparable method is not administered a Tn3 scaffold.

In aspects, a PROMIS-29 assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a PROMIS-29 assessment to a Tn3 scaffold can be assessed on about Day 1, Day 15±1, Day 29±3, Day 57±3, Day 85±3, Day 113±5, Day 141±5, Day 169±5, Day 197±7, Day 225±7, Day 253±7, Day 281±7, and Day 309±7 post treatment initiation.

### RAPID3

In aspects, an assessment comprises a RAPID3 (routine assessment of patient index data 3). RAPID is a pooled index of the 3 patient-reported American College of Rheumatology RA Core Data Set measures: function, pain, and patient global estimate of status. Each of the 3 individual measures may be scored from at least about 0, about 1, about 2, about 3, about 4, about 5, about 6, about 7, about 8, about 9, to about 10, for a maximum total of 30. Disease severity may be classified based on the following RAPID3 scores: >12=high; 6.1-12=moderate; 3.1-6=low; <or =3=remission. In aspects, a RAPID3 scores is correlated with a disease activity score 28 (DAS28) and clinical disease activity index (CDAI), or a combination thereof. In aspects, a treated subject comprises an improved RAPID3 score as compared to an otherwise comparable method wherein the subject of the otherwise comparable method is not administered a Tn3 scaffold.

In aspects, a RAPID3 assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, or at least about 48 weeks post treatment initiation. In aspects, a RAPID3 assessment to a Tn3 scaffold can be assessed on about Day 1, Day 15±1, Day 29±3, Day 57±3, Day 85±3, Day 113±, Day 141±, Day 169±, Day 197±, Day 225±, Day 253±, Day 281±, and Day 309±7 post treatment initiation.

### Pharmaceutical Composition

In aspects, provided is a pharmaceutical composition. A pharmaceutical composition can comprise a Tn3 scaffold and/or a TNFi. In aspects a pharmaceutical composition is part of a therapeutic regimen that comprises a Tn3 scaffold and one or more additional therapeutics provided herein. In aspects, the one or more additional therapeutics can comprise a TNFi.

Many drugs can be administered orally as liquids, capsules, tablets, or chewable tablets. Because the oral route is the most convenient and usually the safest and least expensive, it is the one most often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver before being transported via the bloodstream to its target site. The intestinal wall and liver chemically alter (metabolize) many drugs, decreasing the amount of drug reaching the bloodstream. Consequently, these drugs are often given in smaller doses when injected intravenously to produce the same effect.

For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm.

In aspects, a pharmaceutical composition provided herein is administered via infusion. An infusion can take place over a period of time. For example, an infusion can be an administration of a pharmaceutical over a period of about 5 minutes to about 10 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, 5 hours, 6 hours, 7 hours, 8 hours, 9 hours, or up to about 10 hours.

In aspects, intravenous administration is used to deliver a precise dose quickly and in a well-controlled manner throughout the body. It is also used for irritating solutions, which would cause pain and damage tissues if given by subcutaneous or intramuscular injection. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs must be given by continuous infusion to keep their effect constant. In aspects, infusion reactions can occur and include headache, nausea, somnolence, dyspnea, fever, myalgia, rash, or other symptoms. Potential risks associated with administration of a Tn3 scaffold are infection, redness, swelling, pain, and induration at the administration site. Prior to each IV infusion subjects may receive prophylaxis with IV methylprednisolone, oral diphenhydramine, and oral acetaminophen, or equivalent(s) to reduce the risk or severity of potential reactions.

In aspects, a pharmaceutical is administered intrathecally. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

Drugs administered by inhalation through the mouth can be atomized into smaller droplets than those administered by the nasal route, so that the drugs can pass through the windpipe (trachea) and into the lungs. How deeply into the lungs they go depends on the size of the droplets. Smaller droplets go deeper, which increases the amount of drug absorbed. Inside the lungs, they are absorbed into the bloodstream. Drugs applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In aspects, a treatment regime comprising a pharmaceutical composition may be dosed according to a body weight of a subject. In subjects who are determined obese (BMI>35) a practical weight may need to be utilized. BMI is calculated by BMI=weight (kg)/[height (m)]2. An ideal body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight—ideal body weight)). In aspects, body surface area may be utilized to calculate a dosage. A body surface area (BSA) may be calculated by: BSA (m2)=VHeight (cm)*Weight (kg)/3600.

In aspects, a pharmaceutical composition can be administered either alone or together with a pharmaceutically acceptable carrier or excipient, by any routes, and such administration can be carried out in both single and multiple dosages. More particularly, a pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, pharmaceutical formulations can be suitably sweetened and/or flavored by means of various agents of the type commonly employed for such purposes. Exemplary carriers and excipients can include dextrose, sodium chloride (NaCl), sucrose, lactose, cellulose, xylitol, sorbitol, maltitol, gelatin, PEG, PVP, histidine/histidine hydrochloride, trehalose dihydrate, polysorbate 80, and any combination thereof. In aspects, an excipient comprises: histidine/histidine hydrochloride, NaCl, trehalose dihydrate, and polysorbate 80.

### NUMBERED EMBODIMENTS

Notwithstanding the appended claims, the following numbered embodiments also form part of the instant disclosure.

### Embodiment Set 1

1. A method for treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, wherein the Tn3 scaffold comprising the CD40L-specific monomer subunit is administered at a dose of about 1500 mg, and wherein the Tn3 scaffold is administered once about every 2 weeks for at least 2 doses, and is administered about once a month thereafter.
2. A method for treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 3 doses, and is administered about once a month thereafter.
3. A method for treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 months for at least 2 doses.
4. A method for treating rheumatoid arthritis in a subject in need thereof comprising: administering a Tn3 scaffold comprising a CD40L-specific monomer subunit to the subject; wherein the Tn3 scaffold specifically binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 3000 mg, and wherein the Tn3 scaffold is administered about once, once a month, once about every two months, or once about every three months.
5. The method of any one of embodiments 1-4, wherein the Tn3 scaffold is administered in combination with a second therapy.
6. The method of embodiment 5, wherein the second therapy comprises an anti-inflammatory agent, an anti-pain agent, a Disease-modifying Antirheumatic Drug (DMARD), a corticosteroid, or an immune system modifying agent.
7. The method of embodiment 6, comprising the DMARD, wherein the DMARD is a conventional DMARD or a biologic DMARD.
8. The method of embodiment 7, wherein the DMARD is the conventional DMARD, and wherein the conventional DMARD is selected from the group consisting of: hydroxychloroquine, leflunomide, sulfasalazine, tofacitinib, and combinations thereof.
9. The method of embodiment 7, wherein the DMARD is the biologic DMARD, and wherein the biologic DMARD is selected from the group consisting of: etanercept, infliximab, adalimumab, certolizumab, and golimumab.
10. The method of any of embodiments 1-9, wherein the Tn3 scaffold is administered intravenously.
11. The method of embodiment 10, wherein the intravenous administration comprises an infusion.
12. The method of any one of embodiments 1-11, wherein the Tn3 scaffold comprises two CD40L-specific monomer subunits connected in tandem.
13. The method of any one of embodiments 1-12, wherein the Tn3 scaffold binds CD40L and prevents binding of CD40L to CD40 and/or disrupts CD40 mediated signaling.
14. The method of any one of embodiments 1-13, wherein at least one CD40L-specific monomer subunit is fused or conjugated to a heterologous moiety selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, and a cytokine.
15. The method of any one of embodiments 1-14, wherein at least one CD40L-specific monomer subunit is conjugated to PEG or is fused to a human serum albumin (HSA).
16. The method of embodiment 15, wherein the HSA is a variant HSA comprising the amino acid sequence of SEQ ID NO: 4.
17. The method of any one of embodiments 1-16, wherein the Tn3 scaffold comprises the sequence of SEQ ID NO: 1.
18. The method of any one of embodiments 1-17, wherein the Tn3 scaffold is Dazodalibep.
19. The method of any of embodiments 1-18 wherein a first dose of the Tn3 scaffold is administered at about 13 to about 25 mg/min, a second dose and third dose are administered at about 25 mg/min, and a fourth dose is administered at about 17 to about 33 mg/min.
20. The method of any of embodiments 1-18, wherein a first dose of the Tn3 scaffold is administered at about 8 to about 17 mg/min, and a second dose and third dose are administered at about 13 mg/min.
21. The method of any of embodiments 1-20, wherein a first dose of the Tn3 scaffold is about 1500 mg and subsequent doses are about 1500 mg or about 3000 mg.
22. The method of embodiment 21, wherein the first and a second dose of the Tn3 scaffold are about 1500 mg and the subsequent doses are about 3000 mg.
23. The method of any of embodiments 1-22, wherein a first dose is administered over 120 min or 180 min and subsequent doses are administered over 60 or 90 min.
24. The method of any one of embodiments 4-18, wherein the Tn3 scaffold is administered once.
25. The method of any one of embodiments 4-18, wherein the Tn3 scaffold is administered quarterly.
26. The method of embodiment 25, wherein the quarterly administration of the Tn3 scaffold is equally efficacious or more efficacious than more frequent administrations of the Tn3 scaffold as determined by change from baseline in DAS28-CRP in a treated subject.
27. The method of embodiment 26, wherein the change from baseline of the DAS28-CRP is determined quarterly.
28. The method of embodiment 26, wherein the change from baseline of the DAS28-CRP is determined monthly.
29. The method of embodiment 26, wherein the change from baseline of the DAS28-CRP is determined yearly.
30. The method of embodiment 25, wherein the quarterly administration of the Tn3 scaffold confers sustained treatment efficacy in the subject in need as compared to an otherwise comparable subject undergoing more frequent administrations of the Tn3 scaffold as determined by a treatment assessment, and wherein the sustained treatment efficacy is of at least or at most about 15 days, 1 month, 2 months, 3 months, 4 months, 5 months, 6 months, 8 months, 10 months, 1 year, or 1.5 years.
31. The method of embodiment 30, wherein the treatment assessment is determined quarterly.

### Embodiment Set 2

Embodiment 1. A method for treating rheumatoid arthritis in a subject in need thereof comprising: administering a Tn3 scaffold comprising a CD40L-specific monomer subunit to the subject; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, wherein the Tn3 scaffold comprising the CD40L-specific monomer subunit is administered at a dose of about 1500 mg, and wherein the Tn3 scaffold is administered once about every 2 weeks for at least 2 doses, and is administered about once a month thereafter.
Embodiment 2. A method for treating rheumatoid arthritis in a subject in need thereof comprising: administering a Tn3 scaffold comprising a CD40L-specific monomer subunit to the subject; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 3 doses, and is administered about once a month thereafter.
Embodiment 3. A method for treating rheumatoid arthritis in a subject in need thereof comprising: administering a Tn3 scaffold comprising a CD40L-specific monomer subunit to the subject; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 months for at least 2 doses.
Embodiment 4. A method for treating rheumatoid arthritis in a subject in need thereof comprising: administering a Tn3 scaffold comprising a CD40L-specific monomer subunit to the subject; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 3000 mg, and wherein the Tn3 scaffold is administered about once, once a month, once about every two months, or once about every three months.
Embodiment 5. The method of any one of embodiments 1-4, wherein the Tn3 scaffold is administered in combination with a second therapy.
Embodiment 6. The method of embodiment 5, wherein the second therapy comprises an anti-inflammatory agent, an anti-pain agent, a Disease-modifying Antirheumatic Drug (DMARD), a corticosteroid, or an immune system modifying agent.
Embodiment 7. The method of embodiment 6, wherein the second therapy comprises the DMARD, and wherein the DMARD is a conventional DMARD or a biologic DMARD.
Embodiment 8. The method of embodiment 7, wherein the DMARD is the conventional DMARD, and wherein the conventional DMARD is selected from the group consisting of: hydroxychloroquine, leflunomide, sulfasalazine, tofacitinib, and combinations thereof.
Embodiment 9. The method of embodiment 7, wherein the DMARD is the biologic DMARD, and wherein the biologic DMARD is selected from the group consisting of: etanercept, infliximab, adalimumab, certolizumab, and golimumab.
Embodiment 10. The method of any of embodiments 1-9, wherein the Tn3 scaffold is administered intravenously.
Embodiment 11. The method of embodiment 10, wherein the intravenous administration comprises an infusion.
Embodiment 12. The method of any one of embodiments 1-11, wherein the Tn3 scaffold comprises two CD40L-specific monomer subunits connected in tandem.
Embodiment 13. The method of any one of embodiments 1-12, wherein the Tn3 scaffold binds CD40L and prevents binding of CD40L to CD40 and/or disrupts CD40 mediated signaling.
Embodiment 14. The method of any one of embodiments 1-13, wherein at least one CD40L-specific monomer subunit is fused or conjugated to a heterologous moiety selected from the group consisting of: a protein or portion thereof, a linker, a drug, a toxin, a cytotoxic agent, an imaging agent, a radionuclide, a radioactive compound, an organic polymer, an inorganic polymer, a polyethylene glycol (PEG), biotin, an albumin or binding domain thereof, a human serum albumin FcRn binding portion, an antibody or portion thereof, an enzyme, a ligand, a receptor, a binding peptide, a non-FnIII scaffold, an epitope tag, a recombinant polypeptide polymer, and a cytokine.
Embodiment 15. The method of any one of embodiments 1-14, wherein the CD40L-specific monomer subunit is conjugated to PEG.
Embodiment 16. The method of embodiment 15, wherein the CD40L-specific monomer subunit is fused to a human serum albumin (HSA).
Embodiment 17. The method of embodiment 16, wherein the HSA is a variant HSA comprising the amino acid sequence of SEQ ID NO: 4.
Embodiment 18. The method of any one of embodiments 1-16, wherein the Tn3 scaffold comprises the sequence of SEQ ID NO: 1.
Embodiment 19. The method of any of embodiments 1-18 wherein a first dose of the Tn3 scaffold is administered at about 13 to about 25 mg/min, a second dose and third dose are administered at about 25 mg/min, and a fourth dose is administered at about 17 to about 33 mg/min.
Embodiment 20. The method of any of embodiments 1-18, wherein a first dose of the Tn3 scaffold is administered at about 8 to about 17 mg/min, and a second dose and third dose are administered at about 13 mg/min.
Embodiment 21. The method of any of embodiments 1-20, wherein a first dose of the Tn3 scaffold is about 1500 mg and subsequent doses are about 1500 mg or about 3000 mg.
Embodiment 22. The method of embodiment 21, wherein the first and a second dose of the Tn3 scaffold are about 1500 mg and the subsequent doses are about 3000 mg.
Embodiment 23. The method of any of embodiments 1-22, wherein a first dose is administered over 120 min or 180 min and subsequent doses are administered over 60 or 90 min.
Embodiment 24. The method of any one of embodiments 4-18, wherein the Tn3 scaffold is administered once.
Embodiment 25. The method of any one of embodiments 4-18, wherein the Tn3 scaffold is administered quarterly.
Embodiment 26. The method of embodiment 25, wherein the quarterly administration of the Tn3 scaffold is equally efficacious or more efficacious than more frequent administrations of the Tn3 scaffold as determined by change from baseline in DAS28-CRP in a treated subject.
Embodiment 27. The method of embodiment 26, wherein the change from baseline of the DAS28-CRP is determined quarterly.
Embodiment 28. The method of embodiment 26, wherein the change from baseline of the DAS28-CRP is determined monthly.
Embodiment 29. The method of embodiment 26, wherein the change from baseline of the DAS28-CRP is determined yearly.
Embodiment 30. The method of embodiment 25, wherein the quarterly administration of the Tn3 scaffold confers sustained treatment efficacy in the subject in need as compared to an otherwise comparable subject undergoing more frequent administrations of the Tn3 scaffold as determined by a treatment assessment, and wherein the sustained treatment efficacy is of at least or at most about 15 days, 1 month, 2 months, 3 months, 4 months, 5 months, 6 months, 8 months, 10 months, 1 year, or 1.5 years.
Embodiment 31. The method of embodiment 30, wherein the treatment assessment is determined quarterly.
Embodiment 32. The method of any one of embodiments 1-31, wherein the CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21.
Embodiment 33. The method of any one of embodiments 12-32, wherein the CD40L-specific monomer subunit comprises the polypeptide of SEQ ID NO: 22, and wherein the second CD40L-specific monomer subunit comprises the polypeptide of SEQ ID NO: 25.
Embodiment 34. The method of any one of embodiments 1-11, wherein the CD40L-specific monomer subunit comprises the polypeptide of SEQ ID NO: 22
Embodiment 35. The method of any one of embodiments 1-11, wherein the CD40L-specific monomer subunit comprises the polypeptide of SEQ ID NO: 25.

### Examples

Example 1—A phase 2, randomized, double-blind, placebo-controlled, mechanistic insight and dosage optimization study of the efficacy and safety of Dazodalibep (VIB4920), in subjects with rheumatoid arthritis (RA) Study Design

A multicenter, randomized, double-blind, placebo-controlled, parallel-cohort study to evaluate the safety, efficacy, and PK of VIB4920, an exemplary Tn3 scaffold comprising an anti-CD40L-Tn3 fusion protein, in adults with active, moderate-to-severe adult-onset RA (DAS28 CRP>3.2;≥4 TJC and >4 SJC), and presence of serum RF and/or ACPA who have had an inadequate response to MTX, cDMARD, or an anti-TNFα agent; who are not currently receiving an anti-TNFα agent; and who have had no prior treatment with rituximab or B-cell depletive agents. The effect of VIB4920 on disease activity as assessed by a composite measure in subjects with adult-onset RA is evaluated and/or the tolerability and safety of VIB4920 in subjects with adult-onset RA.

The primary efficacy endpoint was change from baseline to dl 13 in DAS28-CRP. Secondary endpoints included proportions with clinical remission (CR) by DAS28-CRP<2.6) at dl 13, time to rescue medication, change from baseline to dl 13 in RF/ACPAs, treatment-emergent adverse events (AE), serious AE(SAE) and AE of special interest (AESI).

After a screening period of up to 28 days, approximately 75 subjects are randomized in a 1:1:1:1:1 ratio into 5 cohorts:
Cohort 1: VIB4920 1500 mg on Days 1, 15, 29, and 57 (N=15)
Cohort 2: VIB4920 1500 mg on Days 1 and 57, placebo on Days 15 and 29 (N=15)
Cohort 3: VIB4920 3000 mg on Days 1 and 57, placebo on Days 15 and 29 (N=15)
Cohort 4: VIB4920 3000 mg on Day 1 and placebo on Days 15, 29, and 57 (N=15)
Cohort 5: Placebo on Days 1, 15, 29, and 57 (N=15)

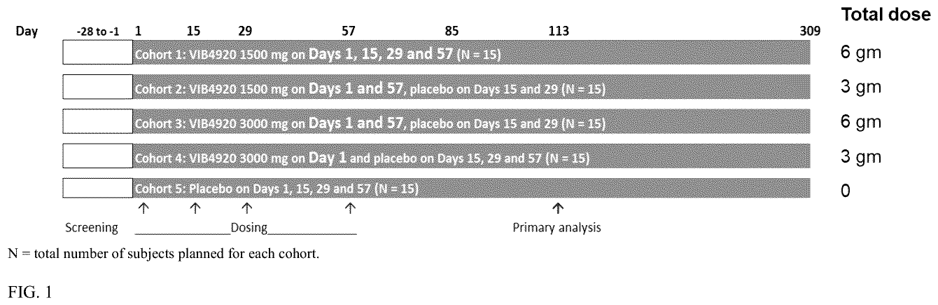

An exemplary study schematic is presented in FIG. 1.

Subjects receive the background therapy that was established for them by their personal physician. Subjects are asked, but not required, to delay institution of any new treatment for RA for 12 weeks (Day 85), at which time rescue therapy may be instituted. Rescue therapy is any new or intensified immunosuppressive, conventional DMARD (cDMARD), or biologic DMARD (bDMARD) treatment for RA, including:
Initiation of or increase in dose of any cDMARD
Initiation of a bDMARD therapy or JAK inhibitor therapy
Increase in baseline corticosteroid dose
Intraarticular steroid injection>40 mg methylprednisolone (or its equivalent) or more than one intraarticular steroid injection of any dose. One intraarticular steroid injection<40 mg methylprednisolone in one joint is permitted and not considered rescue therapy.

If a subject receives rescue therapy prior to the final dose of VIB4920, VIB4920 is discontinued.

Subjects administered rescue therapy do not need to be followed until Day 309 but can complete the study at or after Day 113. Subjects must also complete a 3-month safety follow-up period after the final dose of VIB4920 and must return for at least one visit after initiating rescue therapy to complete remaining assessments (Table 2).

TABLE 2
Alternate Study Completion Schedules Following Rescue Therapy

| Last Dose Prior to | |
|---|---|
| Rescue Therapy | Last Study Visit |
| ≤Dose 3 (Day 29 + 3 d) | Day 113 OR +1 visit post-initiation of |
| | rescue therapy, whichever is later |
| ≥Dose 4 (Day 57 + 3 d) | Day 141 OR +1 visit post-initiation of |
| | rescue therapy, whichever is later |

Note: All actions include the required Day 113 visit, 3 months of follow-up, and one visit post-initiation of rescue therapy.

All subjects are followed at least through the primary (interim) analysis (Day 113), and those who have not instituted rescue therapy are followed through Day 309 to determine the duration of clinical response. The primary analysis is after all subjects have completed Day 113, and the final analysis is after all subjects have completed follow-up.

### Population

### Inclusion Criteria

To be included in the study, each subject must satisfy all the following criteria:
1. Male or female adults, >18 years of age at time of informed consent.
2. Written informed consent and any locally required authorization obtained from the subject prior to performing any protocol-related procedures, including screening evaluations.
3. Diagnosed with RA according to the EULAR/ACR 2010 criteria (Aletaha et al, 2010)>6 months prior to screening.
4. DAS28-CRP>3.2 at screening with≥4 TJC and >4 SJC out of the 28 joints assessed for DAS28 present at screening and confirmed present at Visit 2 prior to randomization.
5. Positive for RF and/or ACPA at screening, in accordance with criteria at the central laboratory.
6. Treated with MTX given orally, SC, or intramuscularly at a dose of 7.5-25.0 mg/week, with or without a concomitant cDMARD other than leflunomide, with MTX and the cDMARD delivered by the same route for >12 weeks without change in dose for >6 weeks prior to screening, OR, if MTX intolerant or if MTX is contraindicated, treated with one or more cDMARD for≥12 weeks without change in dose for >6 weeks prior to screening. (JAK inhibitors are not considered cDMARDs)
7. Willing and able to comply with the protocol, complete study assessments, and complete the study period.
8. Females of childbearing potential who are sexually active with a non-sterilized male partner must use a highly effective method of contraception from signing informed consent and must agree to continue using such precautions through the end of the follow-up of the study; cessation of contraception after this point should be discussed with a responsible physician. Periodic abstinence, the rhythm method, and the withdrawal method are not acceptable methods of contraception. A recommendation that the female partners (of childbearing potential) of male study subjects should use a highly effective method of contraception (Table 3) other than a barrier method is made.

TABLE 3
Highly Effective Methods of Contraception for Females of Childbearing Potential

| Physical Methods | Hormonal Methods |
|---|---|
| Intrauterine device (IUD) | Combined (estrogen and progestogen-containing |
| Intrauterine hormone-releasing system (IUS) ^{a} | hormonal contraception) |
| Bilateral tubal occlusion | Oral (combined pill) |
| Vasectomized partner ^{b} | Injectable |
| Sexual abstinence ^{c} | Transdermal (patch) |
| | Progestogen-only hormonal contraception |
| | associated with inhibition of ovulation ^{d} |
| | Injectable |
| | Implantable |
| | Intravaginal |

^{a} This is also considered to be a hormonal method.
^{b} With appropriate post-vasectomy documentation of surgical success (absence of sperm in ejaculate).
^{c} Sexual abstinence is considered to be a highly effective method only if defined as refraining from heterosexual intercourse during the entire period of the study and if it is the preferred and usual lifestyle of the subject.
^{d} Progestogen-only hormonal contraception, where inhibition of ovulation is not the primary mode of action (minipill) is not accepted as a highly effective method.

a. Females of childbearing potential are defined as those who are not surgically sterile (i.e., surgical sterilization includes bilateral tubal ligation, bilateral oophorectomy, or hysterectomy) or those who are not postmenopausal (defined as 12 months with no menses without an alternative medical cause and a follicle-stimulating hormone within the postmenopausal range as established by the clinical laboratory).
9. Non-sterilized male subjects who are sexually active with a female partner of childbearing potential must use a condom with spermicide from Day 1 through to the end of the study.

### Exclusion Criteria

Any of the following excludes a subject from participation in the study:
1. Prior or current (1) inflammatory joint disease other than RA (e.g., gout, reactive arthritis, psoriatic arthritis, seronegative spondyloarthropathy, Still's disease, or Lyme disease); (2) other systemic autoimmune disorder (e.g., systemic lupus erythematosus, inflammatory bowel disease, scleroderma, inflammatory myopathy, mixed connective tissue disease, or other overlapping syndrome) or polymyalgia rheumatica except that subjects with RA and secondary Sj6gren's syndrome may enroll.
2. Severe interstitial lung disease.
3. Prior receipt of any biologic B-cell-depleting therapy (e.g., rituximab, ocrelizumab, ofatumumab).
4. Receipt of any anti-TNFα biologic agent<8 weeks prior to screening (discontinuation could have been for any reason: lack of efficacy, safety/tolerability issues, or lack of access to drug).
5. Receipt of any bDMARD with a mechanism of action other than direct TNF blockade, including any JAK inhibitor, <12 weeks or <5 half-lives of the drug (whichever is longer) prior to screening.
6. Receipt of any experimental therapy<12 weeks or <5 half-lives of the drug (whichever is longer) prior to screening.
7. Injectable corticosteroids (including intraarticular) or treatment with≥10 mg/day dose of oral prednisolone or equivalent within 4 weeks prior to screening. Concomitant treatment with oral corticosteroids<10 mg/day prednisone or equivalent is permitted provided that the dose is stable for >4 weeks prior to screening and during the screening period and is expected to remain stable for the duration of the treatment period. Inhaled or topical corticosteroids given for asthma, chronic obstructive pulmonary disease, or dermatological conditions are allowed, provided doses are expected to be stable during the study.
8. Previous treatment with anti-CD40L compounds at any time before randomization.
9. History of confirmed deep venous thrombosis or arterial thromboembolism within 2 years of enrollment OR history of recurrent deep venous thrombosis or arterial thromboembolism OR subjects with risk factors for venous thromboembolism or arterial thrombosis (e.g., immobilization or major surgery within 12 weeks before screening), prothrombotic status (including, but not limited to, known congenital or inherited deficiency of antithrombin III, protein C, protein S, or confirmed diagnosis of catastrophic antiphospholipid syndrome).
10. Treatment with anticoagulant drugs (clopidogrel, prasugrel, warfarin, low molecular weight heparin, others). Low-dose aspirin treatment (up to 325 mg/day) is allowed.
11. History of solid organ or cell-based transplantation.
12. Active malignancy or history of malignancy that was active within the last 15 years, except as follows:
   a. In situ carcinoma of the cervix following apparently curative therapy>12 months prior to screening; or
   b. Cutaneous basal cell or squamous cell carcinoma following apparently curative therapy.
13. Pregnancy, lactation, or planning to become pregnant during the duration of the study.
14. Positive test for, or prior treatment for, hepatitis B, hepatitis C, or HIV infection. A positive test for hepatitis B is detection of either (1) hepatitis B surface antigen (HBsAg); or (2) hepatitis B core antibody (anti-HBc).
15. Evidence of active tuberculosis (TB) or being at high risk for TB based on:
   c. History of active TB or untreated/incompletely treated latent TB. Subjects with latent TB who have documentation of completion of treatment according to local guidelines may be enrolled.
   d. History of recent (<12 weeks prior to screening) close contact with someone with active TB (close contact is defined as >4 hours/week OR living in the same household OR in a house where a person with active TB is a frequent visitor).
   e. Signs or symptoms that could represent active TB by medical history or physical examination.
   f Positive, indeterminate or invalid interferon-gamma release assay test result at screening, unless previously adequately treated for latent TB. Subjects with an indeterminate test result can repeat the test once, but if the repeat test is also indeterminate, the subject is excluded.
   g. Chest radiograph that suggests a possible diagnosis of TB or suggests that a work-up for TB should be considered; all subjects must have had a chest radiograph with an acceptable reading within 6 months prior to screening or at screening.
16. History of (a) more than one episode of herpes zoster in the 12 months prior to screening or (b) any opportunistic infection in the 12 months prior to screening, excluding localized mucocutaneous candidiasis.
17. Known history of severe allergy or reaction to any component of the VIB4920 formulation.
18. Severe cardiovascular, respiratory, endocrine, gastrointestinal, hematological, neurological, psychiatric, or systemic disorder or any other condition that, in the opinion of the Investigator, would place the subject at unacceptable risk of complications, interfere with evaluation of the IP, or confound the interpretation of subject safety or study results.

Subjects should be assessed for epidemiologic risk of COVID-19 (recent exposures, high-risk housing) and for health-related risk of COVID-19 severity based on current understanding of risk factors for severe disease when making a decision regarding the subject subject's risk of participation. Subjects who have active COVID-19 infection or disease or other significant infection, or, in the judgment of the investigator, who may be at unacceptable risk of COVID-19 or its complications should not be randomized.

Ensure that the subject has a documented negative SARS-CoV-2 test within two weeks prior to randomization. Subjects with a positive test for SARS-CoV-2 may be rescreened at least 2 weeks after a positive test if asymptomatic and at least 3 weeks after symptomatic COVID-19 illness.
19. Inflammatory osteoarthritis.
20. Receipt of live vaccine or live therapeutic infectious agent within the 4 weeks prior to screening.
21. Blood tests at screening that meet any of the following criteria:

$$Aspartate aminotransferase ( AST ) > 2 \times upper limit of normal ( ULN ) for the central laboratory . \qquad h$$

$$Alanine aminotransferase ( ALT ) > 2 \times ULN . \qquad i$$

$$Total bilirubin ( TBL ) > 2 \times ULN unless AST , ALT , \\ and hemoglobin are within central laboratory normal range and the subject has a known history of Gilbert syndrome . \qquad j$$

$$Hemoglobin < 85 g / L . \qquad k$$

$$Neutrophils < 1.5 \times {10}^{9} / L . \qquad l$$

$$Platelets < 100 \times {10}^{9} / L . \qquad m$$

$$Prothrombin time or partial thromboplastin time ( PTT ) > 1.2 \times ULN . \qquad n$$

22. History of alcohol or drug abuse that, in the opinion of the Investigator, might affect subject safety or compliance with visits, or interfere with safety or other study assessments.

Repeat of study laboratory tests is acceptable in the event that an initial screening safety laboratory result is outside of acceptable limits for the study. If the investigator considers the value to be a potential outlier not representative of the subject's true state of health, testing may be repeated once, using the central laboratory, at the investigator's discretion. Subjects may be rescreened once if, in the Investigator's judgment, the reason for ineligibility is likely to have resolved at the time of rescreening.

Subjects must, based on moderate-to-severe disease despite DMARD therapy, have failed a prior regimen. To avoid confounding interpretation of safety and efficacy data based on the use of prior biologics, especially B-cell depletive agents, subjects with prior use of these drugs, other than prior but not current use of TNFα inhibitors, are excluded. Subjects must have at least 4 tender and 4 swollen joints to permit assessment of joint response during the study.

A complete physical examination (with the exception of rectal and pelvic examinations) is conducted, including vital signs, height, and weight. DAS28-CRP assessments are performed at screening along with testing for RF and ACPA. Laboratory tests (serum chemistry, hematology, and urinalysis), coagulation parameters, chest X-ray (unless recent reading available [prior 6 months]), ECG, and serum human chorionic gonadotropin (β-hCG) pregnancy test for females are performed.

Screening tests comprise: Hepatitis B testing: HbsAg, anti-HBc; Hepatitis C antibody; HIV testing: HIV-1 antibody, HIV-2 antibody; TB testing (e.g., QuantiFERON®-TB Gold Test or other interferon-gamma release assay test) as per local standard of care guidelines.

### Safety Assessments

### Adverse Event (AE)

An AE is any untoward medical occurrence associated with the use of an intervention in humans whether or not it is considered intervention-related.

### Serious Adverse Event (SAE)

A SAE is considered “serious” if it results in any of the following outcomes:
Death
A life-threatening AE. (An event is considered “life-threatening” if, in the view of either the Investigator or Sponsor, its occurrence places the subject or subject at immediate risk of death. It does not include an AE or suspected adverse reaction (SAR) that, had it occurred in a more severe form, might have caused death.)
In subject hospitalization or prolongation of existing hospitalization
A persistent or significant incapacity or substantial disruption of the ability to conduct normal life functions
A congenital anomaly/birth defect
Important medical events that may not result in death, be life-threatening, or require hospitalization may be considered serious when, based upon appropriate medical judgment, they may jeopardize the subject or subject and may require medical or surgical intervention to prevent one of the outcomes listed in this definition. Examples of such medical events include allergic bronchospasm requiring intensive treatment in an emergency room or at home, blood dyscrasias or convulsions that do not result in in subject hospitalization, or the development of drug dependency or drug abuse.

### Causality or Relatedness

An assessment of the relationship of AEs and SAEs to the IP is determined. An event is considered “not related” to use of VIB4920 if any of the following tests are met:
a. An unreasonable temporal relationship between administration of the IP and the onset of the event (e.g., the event occurred either before, or too long after, administration of the IP for it to be considered IP-related)
b. A causal relationship between the IP and the event is biologically implausible (e.g., death as a passenger in an automobile accident)
c. A clearly more likely alternative explanation for the event is present (e.g., typical adverse reaction to a concomitant drug and/or typical disease-related event)

Subject AE/SAE reports are considered “related” to use of the VIB4920 if the “not related” criteria are not met. “Related” implies that the event is considered to be “associated with the use of the drug” meaning that there is “a reasonable possibility” that the event may have been caused by the product (i.e., there are facts, evidence, or arguments to suggest possible causation).

### Severity/Intensity

### Severity is Assessed According to the Following Scale:

a. Grade 1: An event of mild intensity that is usually transient and may require only minimal treatment or therapeutic intervention. The event does not generally interfere with usual activities of daily living.
b. Grade 2: An event of moderate intensity that is usually alleviated with additional, specific therapeutic intervention. The event interferes with usual activities of daily living, causing discomfort but poses no significant or permanent risk of harm to the subject.
c. Grade 3: A severe event that requires intensive therapeutic intervention. The event interrupts usual activities of daily living, or significantly affects the clinical status of the subject.
d. Grade 4: An event, and/or its immediate sequelae, that is associated with an imminent risk of death or with physical or mental disabilities that affect or limit the ability of the subject to perform activities of daily living (eating, ambulation, toileting, etc.).
e. Grade 5: Death (loss of life) as a result of an event.

### Dose Formulation

The formulation of VIB4920 is shown in Table 4.

TABLE 4
Exemplary formulation of VIB4920. The formulation is infused
through a low-protein binding 0.2-0.22 μm filter.

| IPs | Concentration and Formulation as Supplied |
|---|---|
| VIB4920 (500 | VIB4920 for IV administration is supplied as a sterile liquid filled at a nominal |
| mg per vial, | of 5 mL in 6R vials. Each vial contains VIB4920 formulated at 100 mg/mL, in |
| nominal) | 10 mM sodium phosphate buffer, 250 mM sucrose, 0.02% (w/v) poloxamer |
| | 188, pH 7.4 |
| Placebo | 0.9% (w/v) saline |

### Treatment

Treatment administration should, wherever possible, be at a consistent time of day for each dose. Vital signs are obtained prior to the start of each infusion, every 30 (±5) minutes during the infusion, and at the end of the infusion (±5 minutes). Vital signs also are checked every hour (±10 minutes) during the 4-hour observation period after Dose 1 and at the end (±10 minutes) of the one-hour observation period after doses 2, 3, and 4. If vital signs are abnormal, they should be rechecked.

Treatment is infused using an IV infusion pump. Infusion times are as per Table 5. For Doses 1 and 4, IP is either VIB4920 1500 mg, VIB4920 3000 mg, or placebo. For Doses 2 and 3, IP are either VIB4920 1500 mg or placebo. Table 6 shows the study treatment administration scheme.

TABLE 5
Exemplary Infusion Times

| Dose 1, Day 1 | Dose 2, Day 15 | Dose 3, Day 29 | Dose 4, Day 57 |
|---|---|---|---|
| Infusion Time | Infusion Time | Infusion Time | Infusion Time |
| (VIB4920 mg/min)^{a, b} | (VIB4920 mg/min)^{a, c} | (VIB4920 mg/min)^{a} | (VIB4920 mg/min)^{a} |
| 120 min | 60 min | 60 min | 90 min |
| (~13-25 mg/min) | (25 mg/min) | (25 mg/min) | (~17-33 mg/min) |

SDMC = Safety Data Monitoring Committee.
^{a}Or placebo matched to that dose of VIB4920.
^{b}If, after dosing of at least 4 subjects with Dose 1, >25% of the subjects dosed have a Grade 2 or higher infusion reaction, all subsequent doses that could include 3000 mg (i.e., Dose 4) is administered over 180 minutes(~8-17 mg/min) and Doses 2 and 3 is administered over 120 minutes (~13 mg/min).
^{c}If >25% of the subjects dosed have a Grade 2 or higher infusion reaction, Dose 3 is administered over 120 minutes and Dose 4 is administered over 180 minutes.

TABLE 6
Exemplary Treatment Scheme

| Cohort | Dose 1, Day 1 | Dose 2, Day 15 | Dose 3, Day 29 | Dose 4, Day 57 |
|---|---|---|---|---|
| Cohort 1 | VIB4920 1500 mg | VIB4920 1500 mg | VIB4920 1500 mg | VIB4920 1500 mg |
| Cohort 2 | VIB4920 1500 mg | Placebo | Placebo | VIB4920 1500 mg |
| Cohort 3 | VIB4920 3000 mg | Placebo | Placebo | VIB4920 3000 mg |
| Cohort 4 | VIB4920 3000 mg | Placebo | Placebo | Placebo |
| Cohort 5 | Placebo | Placebo | Placebo | Placebo |

Subjects who have had an infusion-related reaction of Grade 1 or Grade 2 may be premedicated prior to subsequent dosing with antihistamines and/or acetaminophen in accordance with doses in the package instructions.

### Post-Treatment Assessment

To evaluate the effect of VIB4920 on disease activity as assessed by a composite measure in subjects with adult-onset RA and to evaluate the safety and tolerability of VIB4920 in subjects with adult-onset RA, post-treatment, subjects are assessed for one or more of the following:
change in DAS28-CRP from baseline to Day 113;
The incidence of treatment-emergent adverse events (TEAEs) and treatment-emergent serious adverse events and TEAEs of special interest during the study

To evaluate the duration of clinical response to VIB4920 as assessed by time to institution of rescue therapy and to characterize the pharmacokinetics (PK) of VIB4920, evaluate the pharmacodynamic effect of VIB4920, evaluate the immunogenicity of VIB4920, evaluate the effect of VIB4920 on autoantibodies, and assess the effect of VIB4920 on clinical remission as assessed by a composite measure in subjects with adult-onset RA, subjects are assessed for one or more of the following:
PK, PD, and ADA of VIB4920
The time-concentration profile of total soluble CD40L
The proportion of subjects with anti-drug antibodies to VIB4920 (immunogenicity of VIB4920)
Change in RF and ACPAs from baseline to Day 113
The proportion of subjects with clinical remission defined as DAS28-CRP≤2.6 at Day 113
Time to start of new treatment for RA (rescue medication; duration of clinical response to VIB4920)

To evaluate the effect of VIB4920 on markers of inflammation; immune cell populations and cytokines, RNA, DNA, and protein biomarkers; subject outcomes as recorded in subject-reported outcome (PRO) instruments; and on additional measures of efficacy, and to evaluate the duration of clinical response to VIB4920 in subjects who have had a clinical response subjects are assessed for one or more of the following:
Change from baseline in levels of markers of inflammation (e.g., immunoglobulins)
Change from baseline in immune cell populations, cytokines, blood gene expression, and protein biomarkers
Change from baseline to Day 113 in 2 PROs: the Health Assessment Questionnaire (HAQ) and the Functional Assessment of Chronic Illness Therapy (FACIT)—Fatigue Scale, duration of clinical remission based on DAS28-CRP≤2.6, Clinical Disease Activity Index (CDAI)<2.8, or Simplified Disease Activity Index (SDAI)≤3.3
Change from baseline in 28 tender joint count (TJC) and 28 swollen joint count (SJC)
Change from baseline in CDAI
The proportion of subjects with low disease activity defined as DAS28-CRP≤3.2 and an improvement of DAS28-CRP score>0.6 from baseline
The proportion of subjects with remission defined as CDAI≤2.8 or SDAI≤3.3
Change from baseline in the physician global assessment of disease activity (MDGA)
Change from baseline in the patient global assessment of disease activity (PGA)
Change from baseline in serum biomarkers of disease activity: RF isotypes, CRP, IL-6, and other biomarkers
Change from baseline in CXCL13
Relationship between genetic data and efficacy
Change from baseline in RNA and DNA

TABLE 7
Summary of Composite Disease Scores and Assessments

| | Composite Disease Scores | | |
|---|---|---|---|
| Assessment | DAS28-CRP | CDAI | SDAI |
| TJC (0-28) | X | X | X |
| SJC (0-28) | X | X | X |
| MDGA (0-100) | | X | X |
| PGA (0-100) | X | X | X |
| CRP (mg/dL) | X | | X |

CDAI = Clinical Disease Activity Index; CRP = C-reactive protein; DAS28-CRP = Disease Activity Score in 28 Joints Using C-reactive Protein; MDGA = physician's global assessment; PGA = patient's global assessment; SDAI = Simplified Disease Activity Index SJC = swollen joint count of 28 joints; TJC = tender joint count of 28 joints.
DAS28-CRP = 0.56 × √(TJC28) + 0.28 × √(SJC28) + 0.014 × PGA(0-100 mm VAS) + 0.36 × In[CRP(mg/L) + 1] + 0.96
CDAI = TJC28 + SJC28 + PGA (0-10 cm VAS) + MDGA (0-10 cm VAS)
SDAI = TJC28 + SJC28 + PGA (0-10 cm VAS) + MDGA (0-10 cm VAS) + CRP (mg/dL)

### Clinical Laboratory

Post-treatment, blood, urine and respiratory (swab or saliva) samples are collected for laboratory safety tests. A hematology panel includes a complete blood count, with white blood cell count (WBC) and differential (basophils, eosinophils, lymphocytes, monocytes, and neutrophils), hemoglobin, hematocrit, and platelet count. Serum chemistry will also be analyzed for:

Albumin
Alkaline phosphatase (ALP)
ALT
AST
Bicarbonate
Blood urea nitrogen
CRP
Calcium
Chloride
Cholesterol
Creatinine
Gamma-glutamyl transferase
Glucose (random)
HbA1C
Immunoglobulins: Total, IgA, IgG, and IgM
Magnesium
Phosphorus
Potassium
Sodium
TBL (if >1.5 ULN, indirect and direct bilirubin is measured)
Total protein
Triglycerides (fasting not required unless abnormal at baseline, in which
case an 8-hour fast is required)
Uric acid

Coagulation parameters will also be assessed: prothrombin time and PTT.

Urinalysis evaluates color, appearance, and specific gravity. Dipstick analysis includes pH, protein, glucose, blood, ketones, and bilirubin. Samples with abnormal dipstick will have microscopy performed. Microscopy includes WBC/HPF (high power field) and red blood cell count/HPF.

Testing for SARS-COV-2 is performed.

Vital signs, including systolic and diastolic blood pressure (mmHg), pulse rate (beats/min), respiratory rate (breaths/min), body temperature (° C.), and body weight (kg), are measured.

An electrocardiogram (ECG) is performed. ECG analysis includes ventricular heart rate and intervals (PR, QRS, QT, QTc).

TABLE 8
Exemplary Schedule of Study Assessments and Procedures

| | Visit number | | | | | | | | | | | | |
|---|---|---|---|---|---|---|---|---|---|---|---|---|---|
| | | | | | | | | | | | | | V14 or |
| | V2 | V3 | V4 | V5 | V6 | V7 | V8 | V9 | V10 | V11 | V12 | V13 | EDV |
| | Study Day Procedure | | | | | | | | | | | | |
| | 1 | 15 ± 1d | 29 ± 3d | 57 ± 3d | | | 141 ± | 169 ± | 197 ± | 225 ± | 253 ± | 281 ± | 309 ± |
| | Dose 1 | Dose 2 | Dose 3 | Dose 4 | 85 ^{g} ± 3d | 113 ± 5d | 5d | 5d | 7d | 7d | 7d | 7d | 7d |
| Assessment of | X | X | X | X | X | X | X | X | X | X | X | X | X |
| AEs/SAEs/AESIs | | | | | | | | | | | | | |
| Concomitant medications | X | X | X | X | X | X | X | X | X | X | X | X | X |
| Patient global assessment of | X | X | X | X | X | X | X | X | X | X | X | X | X |
| disease activity | | | | | | | | | | | | | |
| Completion of HAQ and | X | X | X | X | X | X | X | X | X | X | X | X | X |
| FACIT-Fatigue Scale | | | | | | | | | | | | | |
| ECG | X | | | | X | | | | | | | | X |
| Vital Signs | X ^{e} | X ^{e} | X ^{e} | X ^{e} | X | X | X | X | X | X | X | X | X |
| Weight | X | | | | X | | | | | | | | X |
| Full physical examination | X | | | | X | | | | | | | | X |
| Symptom-driven physical | | X | X | X | | X | X | X | X | X | X | X | |
| examination | | | | | | | | | | | | | |
| 28-joint assessment including | X | X | X | X | X | X | X | X | X | X | X | X | X |
| TJC and SJC | | | | | | | | | | | | | |
| Physician global assessment | X | X | X | X | X | X | X | X | X | X | X | X | X |
| of disease activity | | | | | | | | | | | | | |
| DAS28-CRP for RA, CDAI, | | X | X | X | X | X | X | X | X | X | X | X | X |
| and SDAI (calculated) | | | | | | | | | | | | | |
| Urine pregnancy test ^{a} | X | X | X | X | X | X | X | X | X | X | X | X | X |
| Urinalysis | X | X | X | X | X | X | X | X | X | X | X | X | X |
| Safety labs (chemistry, | X | X | X | X | X | X | X | X | X | X | X | X | X |
| hematology, coagulation) | | | | | | | | | | | | | |
| Total sCD40L (plasma) | X | X | X | X | X | X | X | X | X | X | X | X | X |
| Autoantibody panel | X | X | X | X | X | X | X | X | X | X | X | X | X |
| (RF, RF isotypes) | | | | | | | | | | | | | |
| Biomarkers-other (serum) | X | X | X | X | X | X | X | X | X | X | X | X | X |
| CRP | X | X | X | X | X | X | X | X | X | X | X | X | X |
| IgM, IgG, IgA | X | X | X | X | X | X | X | X | X | X | X | X | X |
| Flow cytometry sample | X | X | X | X | X | X | X | X | X | X | | | X |
| DNA PAXgene (optional, | X | | | | X | | | X | | | | | X |
| with consent) | | | | | | | | | | | | | |
| RNA PAXgene | X | X | X | X | X | X | X | X | | | X | | X |
| Plasma PK sample ^{b} | X ^{f} | X ^{f} | X ^{f} | X ^{f} | X | X | X | X | X | X | | | |
| ADA (plasma) | X | X | X | | X | | | X | | | X | | X |
| Biomarker samples (plasma | X | X | X | X | X | X | X | X | X | X | X | X | X |
| and serum) | | | | | | | | | | | | | |
| PBMC collection ^{c} | X | X | | | X | | | X | | | | | X |
| Verify eligibility criteria | X | | | | | | | | | | | | |
| Randomization | X | | | | | | | | | | | | |
| IP (VIB4920/placebo) | X | X | X | X | | | | | | | | | |
| administration ^{d, e} | | | | | | | | | | | | | |

ACPA = antibodies to citrullinated peptides;
ADA = anti-drug antibody;
AE = adverse event;
AESI = adverse event of special interest;
CDAI = Clinical Disease Activity Index;
cDMARD = conventional disease-modifying anti-rheumatic drug;
ECG = electrocardiogram;
EDV = early discontinuation visit;
FACIT = Functional Assessment of Chronic Illness Therapy;
HAQ = Health Assessment Questionnaire;
Ig = immunoglobulin;
IP = investigational product;
MBDA = multi-biomarker disease activity test;
MTX = methotrexate;
PBMC = peripheral blood mononuclear cells;
PK = pharmacokinetics;
RA = rheumatoid arthritis;
RF = rheumatoid factor;
SAE = serious adverse event;
sCD40L = soluble CD40 ligand;
SDAI = Simplified Disease Activity Index;
SJC = swollen joint count;
TJC = tender joint count;
V = visit.
^{a} In females of childbearing potential; result must be negative prior to dosing.
^{b} On study days when IP is not administered, only one plasma sample for PK is required to be collected at a consistent time across the different study days.
^{c} Whole blood is collected on indicated days for processing to PBMCs.
^{d} IP administration should, wherever possible, be at a consistent time of day for each dose. All procedures and blood sampling, except for postdose PK, must be performed before IP administration.
^{e} Vital signs are obtained prior to the start of each IP infusion, every 30 (± 5) minutes during the infusion, and at the end of the infusion (+ 5 minutes). Vital signs also are checked every hour (± 10 minutes) during the 4-hour observation period after Dose 1 and at the end (+ 10 minutes) of the one-hour observation period after Doses 2, 3 and 4. If vital signs are abnormal, they should be repeated.
^{f} Plasma samples for PK of VIB4920 are collected predose (within 30 minutes prior to start of infusion), and within 10 minutes of the end of infusion.
^{g} After the Day 85 visit, the dose of background cDMARDs and corticosteroids may be adjusted or a new cDMARD may be added (except that MTX and leflunomide may not be used concurrently and rituximab may not be added without discontinuation of VIB4920) if it is clinically indicated to improve disease management.

### Results

78 subjects were randomized (1:1:1:1:1) in a placebo-controlled, parallel design. The anti-CD40L-Tn3 fusion protein (VIB4920) was VIB4920. The groups were placebo, VIB4920 3000 mg×1, VIB4920 1500 mg×2, VIB4920 3000 mg×2, and VIB4920 1500 mg×4, as described in the cohorts above and in FIG. 1. All subjects were dosed 4 times, on about Day 1, 15, 29 and 57-doses that are not VIB4920 are placebo.

As described above, the study population included adults with active, moderate-to-severe adult-onset RA (DAS28-CRP>3.2; ≥4 tender and >4 swollen joints) and presence of serum rheumatoid factor (RF) and/or anti-citrullinated protein antibodies (ACPAs) with inadequate response to MTX, cDMARD, or TNFi agent and no prior treatment with rituximab or B cell depletive agents.

All subjects in this analysis completed Day 113 or discontinued prior to Day 113. All data up to the cut-off date are included in this analysis.

73 subjects (93.6%) completed treatment and 65 (83%) completed the study. At baseline, mean(SD) age was 56 (13) years, 80% female. Demographics and disease characteristics were similar across arms, except for RF+proportion and mean CRP (Table 9). The primary endpoint (DAS28-CRP change from baseline) was met (Table 9) at all doses at dl 13. Observed treatment effect was consistently maintained for cohort 3 through study end. Prolonged responses, beyond dl 13 were also observed for other doses most notably for cohort 1. RF levels decreased significantly vs placebo (PBO) starting d57 to d113 (p<0.0035) for all doses. There was a similar trend with ACPA levels that was significant with cohorts 1 and 4. DAS28-CRP CR rates were similar in all groups but fewer DAZO patients had high disease activity (DAS28-CRP>5.1) at dl 13. Time-to-rescue medication did not differ.

Patients with≥1AE were numerically higher with DAZO vs PBO (74% vs 63%); 3 patients had 4 SAEs in the DAZO group vs none in PBO: 1 nephrolithiasis (discontinued study), 1 COVID-19 infection and 1 patient was hospitalized for COVID and died from unknown cause 2 days after hospital discharge (232d after last dose), all were deemed unrelated to study drug. 11/62 (18%) vs 4/16 (25%) patients had >1AE deemed related to DAZO and PBO, respectively.

The subject status, baseline characteristics, and drug exposure for subjects in placebo, VIB4920 3000 mg×1, VIB4920 1500 mg×2, VIB4920 3000 mg×2, and VIB4920 1500 mg×4 groups are reported in Tables 9-11.

TABLE 9
Baseline characteristics

| | Dazodalibep | Dazodalibep | Dazodalibep | Dazodalibep | | |
|---|---|---|---|---|---|---|
| | 1500 mg × 4 | 1500 mg × 2 | 3000 mg × 2 | 3000 mg × 1 | Total for | Placebo, |
| | N = 15 | N = 16 | N = 15 | N = 15 | Dazodalibep | N = 16 |
| BL DAS28-CRP | 5.8 (0.7) | 5.9 (0.7) | 5.5 (0.6) | 5.5 (0.9) | — | 5.4 (1.1) |
| BL CRP, mg/L | 14.1 (13.9) | 14.9 (16.1) | 8.8 (9.2) | 7.2 (12.4) | | 17.6 (23.7) |
| BL RF-positive, n | 8 (53) | 15 (94) | 12 (80) | 12 (75) | | 14 (88) |
| (%) | | | | | | |

TABLE 10
RA Related Medications at Baseline

| | | | VIB4920 | | VIB4920 | | VIB4920 | | VIB4920 | |
|---|---|---|---|---|---|---|---|---|---|---|
| | Placebo | | 3000 MG × 1 | | 1500 MG × 2 | | 3000 MG × 2 | | 1500 MG × 4 | |
| | (N = 16) | | (N = 16) | | (N = 16) | | (N = 15) | | (N = 15) | |
| Glucocorticoids | 8 | (50.0%) | 7 | (43.8%) | 7 | (43.8%) | 6 | (40.0%) | 5 | (33.3%) |
| Methylprednisolone | 5 | (31.3%) | 6 | (37.5%) | 4 | (25.0%) | 4 | (26.7%) | 4 | (26.7%) |
| Prednisone | 3 | (18.8%) | 1 | (6.3%) | 3 | (18.8%) | 2 | (13.3%) | 1 | (6.7%) |
| Prednisone equivalent, mg/day | | | | | | | | | | |
| Mean(SD) | 6.9 | (2.6) | 5.5 | (3.4) | 5.4 | (0.9) | 7.5 | (2.7) | 5.0 | (0) |
| cDMARDs and cytotoxics | 16 | (100%) | 16 | (100%) | 16 | (100%) | 15 | (100%) | 15 | (100%) |
| Chloroquine phosphate | 0 | | 1 | (6.3%) | 0 | | 0 | | 0 | |
| Hydroxychloroquine | 1 | (6.3%) | 1 | (6.3%) | 1 | (6.3%) | 0 | | 0 | |
| Leflunomide | 0 | | 0 | | 1 | (6.3%) | 0 | | 1 | (6.7%) |
| Sulfasalazine | 0 | | 1 | (6.3%) | 0 | | 3 | (20.0%) | 0 | |
| Methotrexate | 15 | (93.8%) | 14 | (87.5%) | 15 | (93.8%) | 12 | (80.0%) | 14 | (93.3%) |
| Oral | 12 | (75.0%) | 10 | (62.5%) | 13 | (81.3%) | 10 | (66.7%) | 11 | (73.3%) |
| SubQ | 3 | (18.8%) | 4 | (25.0%) | 2 | (12.5%) | 2 | (13.3%) | 3 | (20.0%) |
| Mean (SD) | 19.7 | (4.8) | 16.6 | (3.6) | 17.3 | (4.9) | 16.7 | (4.4) | 16.8 | (4.3) |

TABLE 11
Exposure to Study Drug

| | | | VIB4920 | | VIB4920 | | VIB4920 | | VIB4920 | |
|---|---|---|---|---|---|---|---|---|---|---|
| | Placebo | | 3000 MG × 1 | | 1500 MG × 2 | | 3000 MG × 2 | | 1500 MG × 4 | |
| | (N = 16) | | (N = 16) | | (N = 16) | | (N = 15) | | (N = 15) | |
| Number of doses received | | | | | | | | | | |
| 1 | 0 | | 0 | | 0 | | 1 | (6.7%) | 1 | (6.7%) |
| 3 | 1 | (6.3%) | 1 | (6.3%) | 1 | (6.3%) | 2 | (13.3%) | 0 | |
| 4 | 15 | (93.8%) | 15 | (93.8%) | 15 | (93.8%) | 12 | (80.0%) | 14 | (93.3%) |
| Duration of exposure (Days^{)a} | | | | | | | | | | |

| Mean | 84.4 | 84.5 | 83.9 | 79.7 | 81.8 |
|---|---|---|---|---|---|
| SD | 8.1 | 5.3 | 7.9 | 16.2 | 14.8 |
| Median | 85.5 | 85 | 85 | 85 | 85 |
| (Min, Max) | (57, 99) | (66, 92) | (57, 98) | (29, 92) | (29, 92) |

^{a}Duration of exposure = last dose date + 28 − first dose date + 1

### Efficacy Data

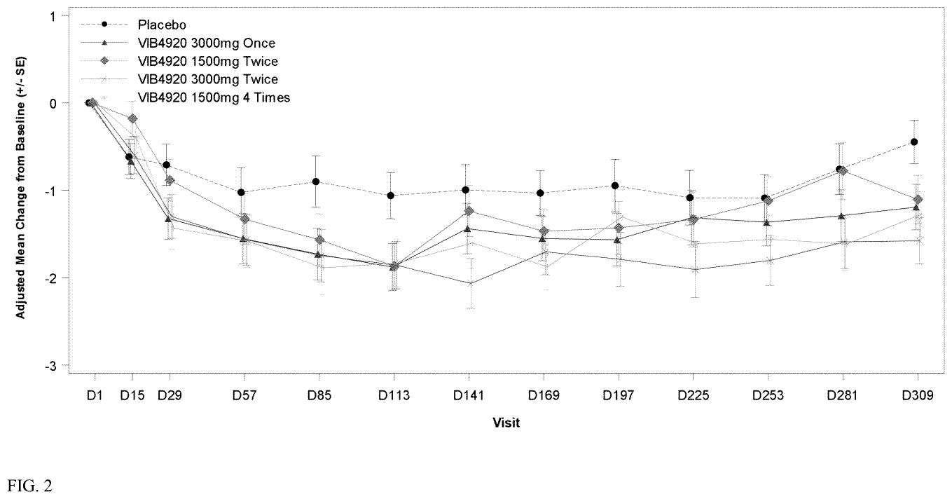

All 4 VIB4920 doses (VIB4920 3000 mg×1, VIB4920 1500 mg×2, VIB4920 3000 mg×2, and VIB4920 1500 mg×4) met statistical significance in the change from baseline DAS28-CRP at Day 113. No dose-response relationship was observed (FIG. 2). A single administration of a Tn3 scaffold is as efficacious or more efficacious than two or more administrations. The data show that about 3000 mg Q8W (administered twice) is about as efficacious as 1500 mg Q4W, and the 3000 mg administered once is also efficacious. Unexpectedly, these data support a longer dosing interval.

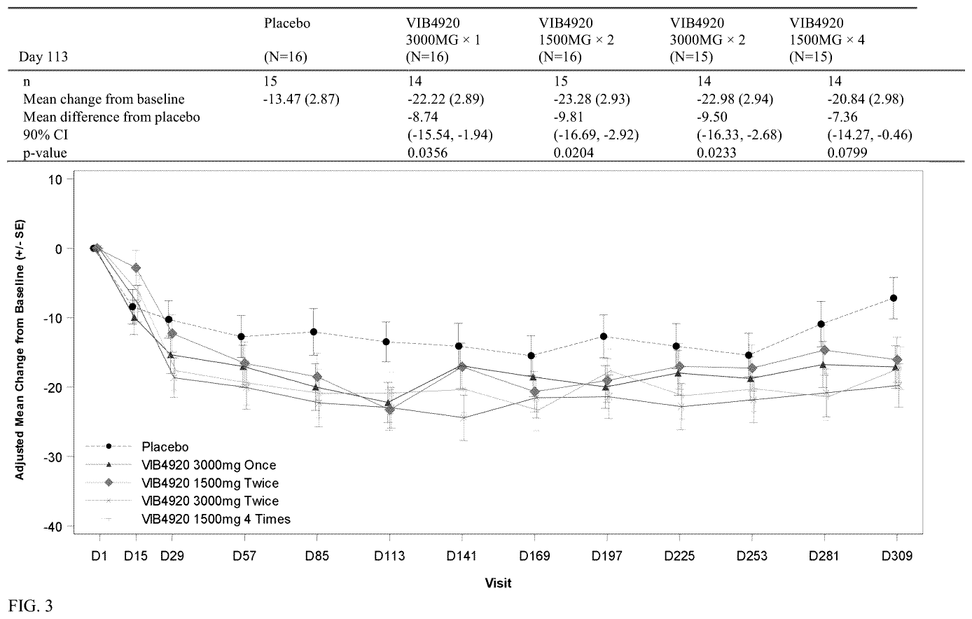

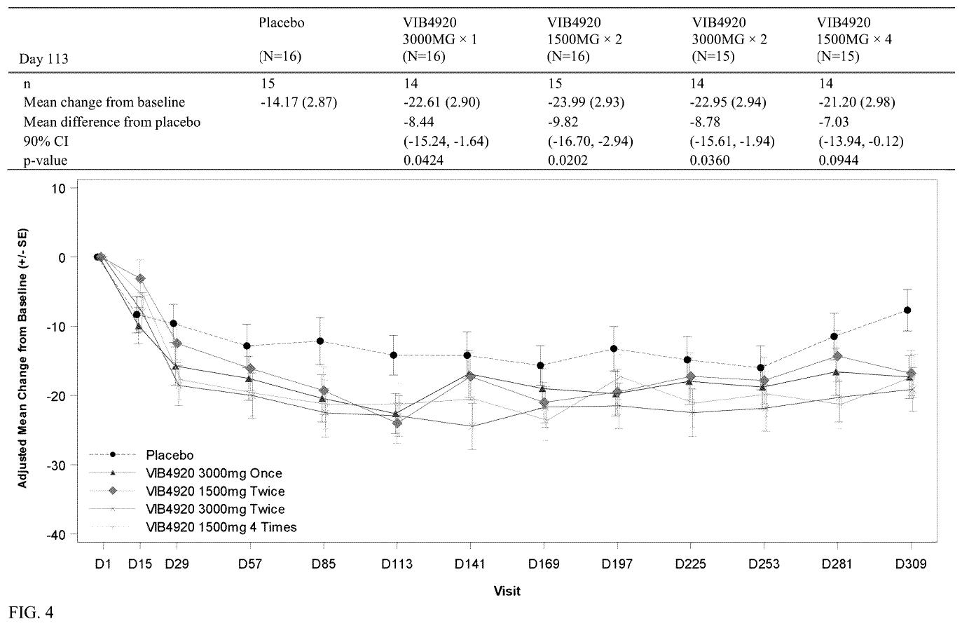

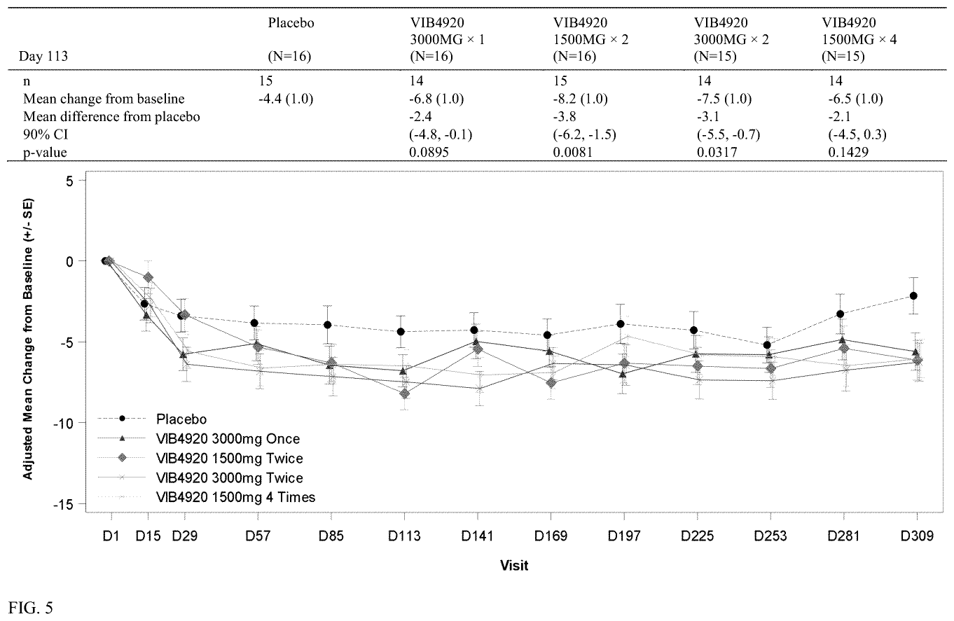

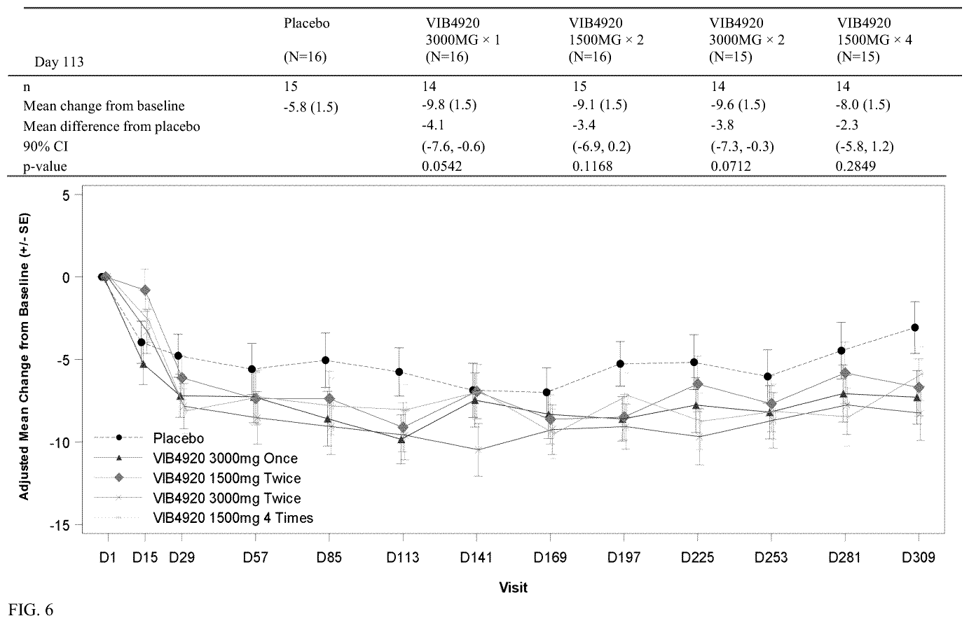

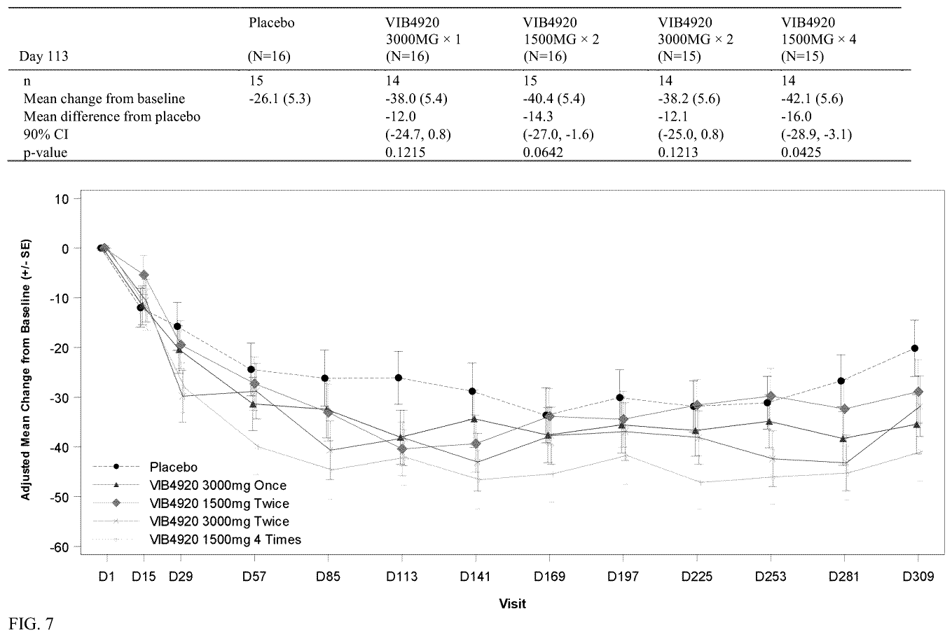

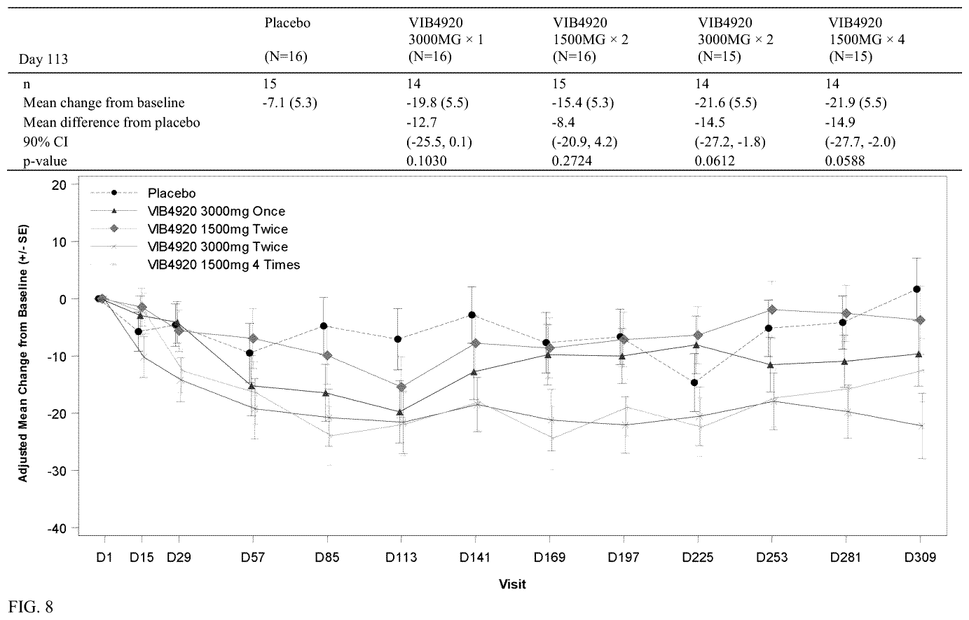

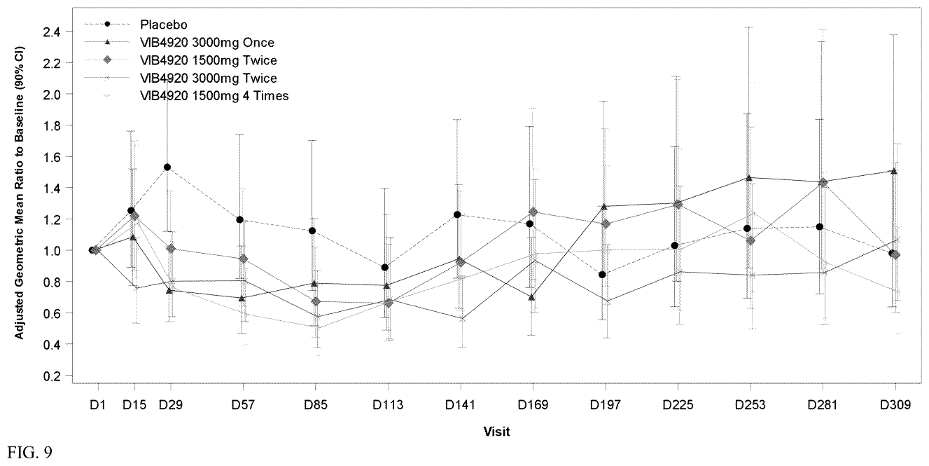

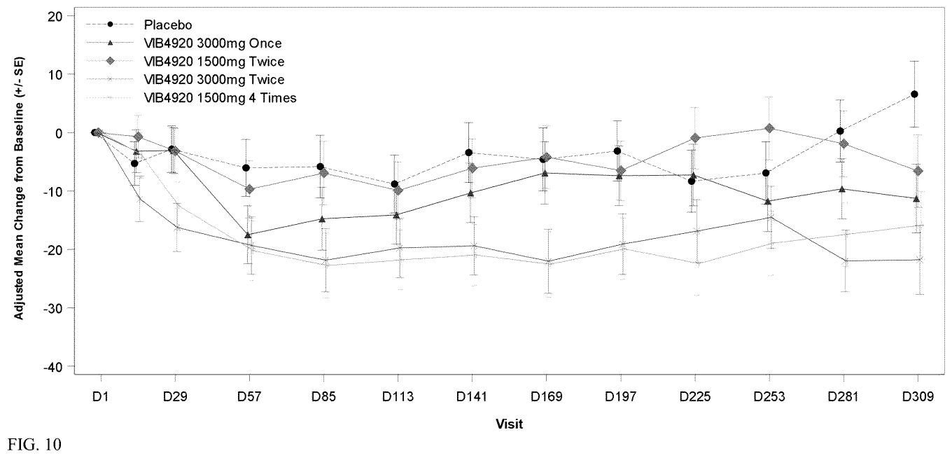

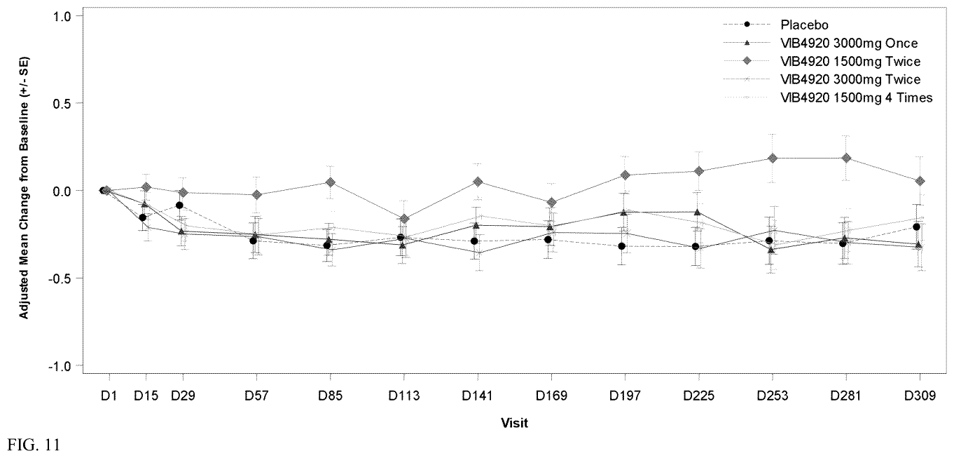

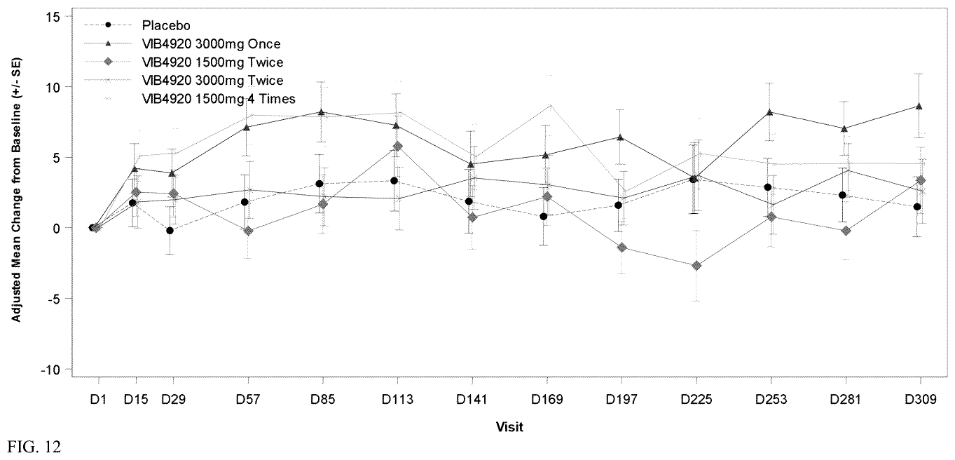

Data from other endpoints including CDAI (FIG. 3), SDAI (FIG. 4), SJC (FIG. 5), TJC (FIG. 6), MDGA (FIG. 7), PtGA (FIG. 8), Pain (FIG. 10) support the DAS28-CRP result.

Table 12 shows the change from Baseline DAS28-CRP to Day 113 and Table 13 shows DAS28-CRP Response Categories for subjects in placebo, VIB4920 3000 mg×1, VIB4920 1500 mg×2, VIB4920 3000 mg×2, and VIB4920 1500 mg×4 groups.

TABLE 12
Final change from baseline DAS28-CRP to Day 113

| | VIB4920 | VIB4920 | VIB4920 | VIB4920 | | |
|---|---|---|---|---|---|---|
| | 1500 mg × 4 | 1500 mg × 2 | 3000 mg × 2 | 3000 mg × 1 | Total for | PBO, |
| | N = 15 | N = 16 | N = 15 | N = 15 | VIB4920 | N = 16 |
| ADAS28- | −1.83 | −1.87 | −1.87 | −1.90 | n/a | −1.06 |
| CRP, BL to | (0.28)**; | (0.27)**; | (0.27)**; | (0.27)**; | | (0.26); |
| d113, LS | n = 14 | n = 15 | n = 14 | n = 14 | | n = 15 |
| mean (SE), | | | | | | |
| MMRM | | | | | | |
| DAS28- | 2 (13) | 1 (6) | 2 (13) | 3 (19) | | 3 (19) |
| CRP < 2.6, n | | | | | | |
| (%) | | | | | | |
| ΔRF, BL to | 0.57 | 0.74 | 0.72 | 0.77 | | 1.20 |
| d113 † | (0.49, 0.66) | (0.64, 0.86) | (0.62, 0.84) | (0.66, 0.89) | | (1.04, 1.39) |
| ΔACPA, BL | 0.62 | 0.82 | 0.84 | 0.69 | | 1.08 |
| to d113 † | (0.47, 0.82)* | (0.62, 1.07) | (0.63, 1.11) | (0.52, 0.91)* | | (0.83, 1.42) |
| Time-to- | 1.04 | 1.04 | 3.01 | 1.04 | | — |
| rescue med; | (0.1, 10.60) | (0.1, 10.60) | (0.45, 20.09) | (0.1, 10.60) | | |
| HR(90% CI) | | | | | | |

TABLE 13
Preliminary DAS28-CRP Response Categories

| | | VIB4920 | VIB4920 | | VIB4920 | | VIB4920 |
|---|---|---|---|---|---|---|---|
| | Placebo | 3000 MG × 1 | 1500 MG × 2 | | 3000 MG × 2 | | 1500 MG × 4 |
| Day 113 | (N = 16) | (N = 16) | (N = 16) | | (N = 15) | | (N = 15) |
| n | 15 | 14 | 15 | | 14 | | 14 |
| Clinical Remission (<2.6) | 3 (20.0%) | 3 (21.4%) | 1 | (6.7%) | 2 | (14.3%) | 2 (14.3%) |
| Low disease activity (>=2.6 to <=3.2) | 2 (13.3%) | 3 (21.4%) | 2 | (13.3%) | 2 | (14.3%) | 3 (21.4%) |
| Moderate disease activity (>3.2 to <=5.1) | 4 (26.7%) | 6 (42.9%) | 10 | (66.7%) | 9 | (64.3%) | 7 (50.0%) |
| High disease activity (>5.1) | 6 (40.0%) | 2 (14.3%) | 2 | (13.3%) | 1 | (7.1%) | 2 (14.3%) |

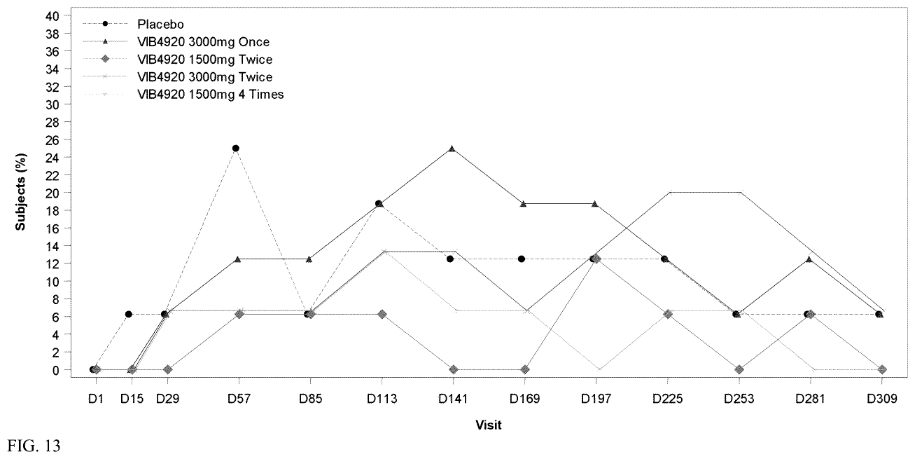

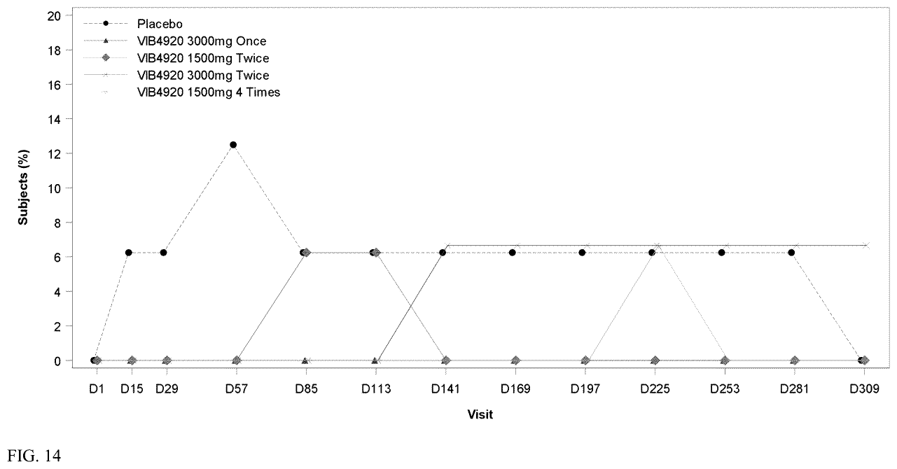

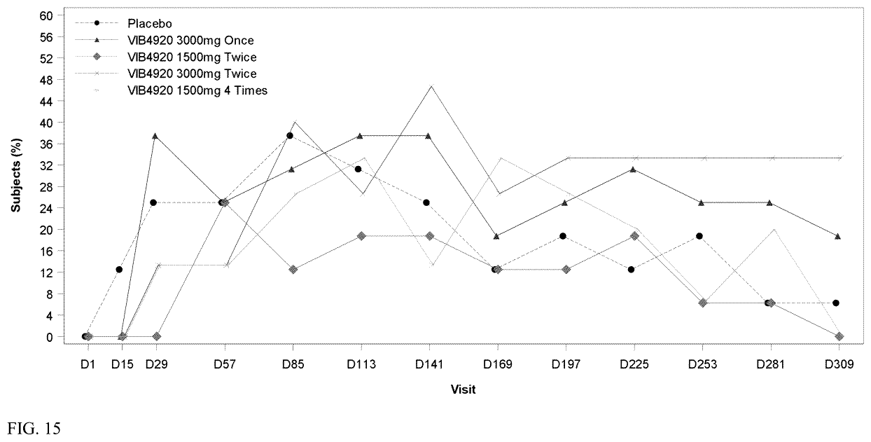

Clinical remission data is shown in FIGS. 13 and 14. Subjects achieving low disease activity are shown in FIG. 15.

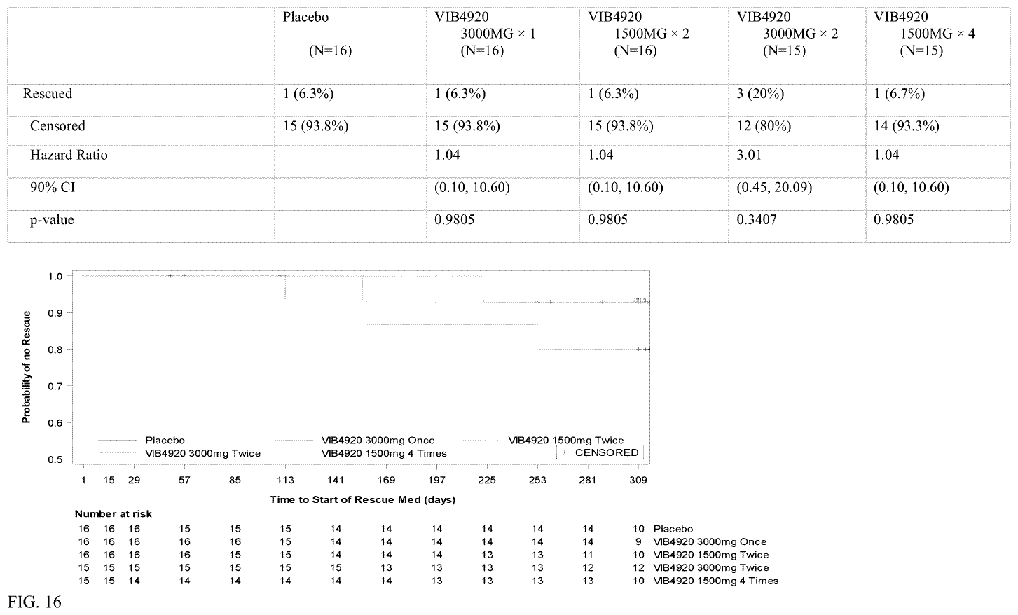

No subject received rescue medications prior to Day 113, and few subjects overall received rescue medications (FIG. 16). Data were insufficient to assess any effect of dosing on time to rescue.

### Biomarkers

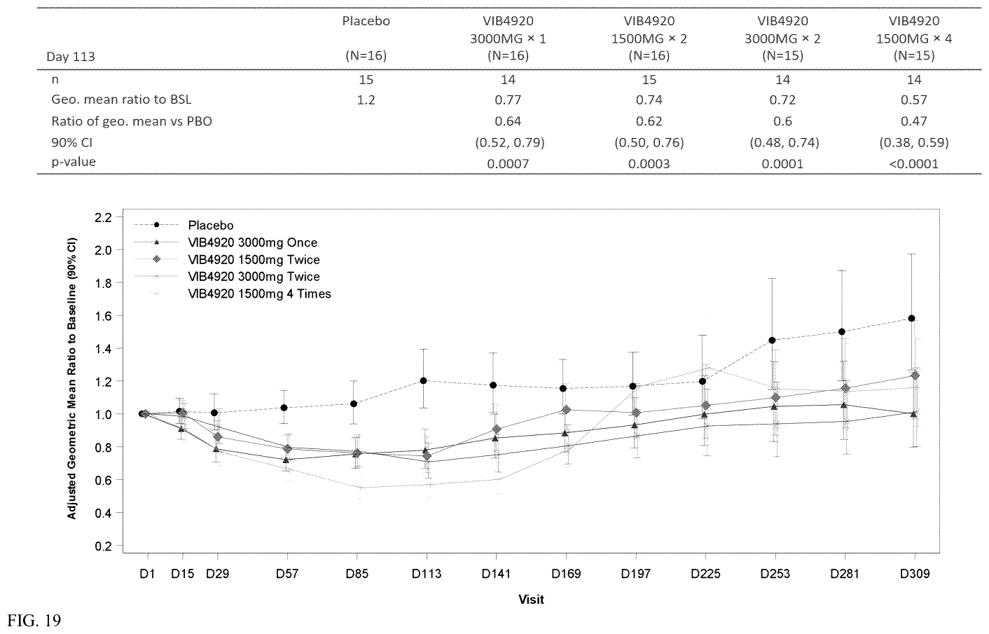

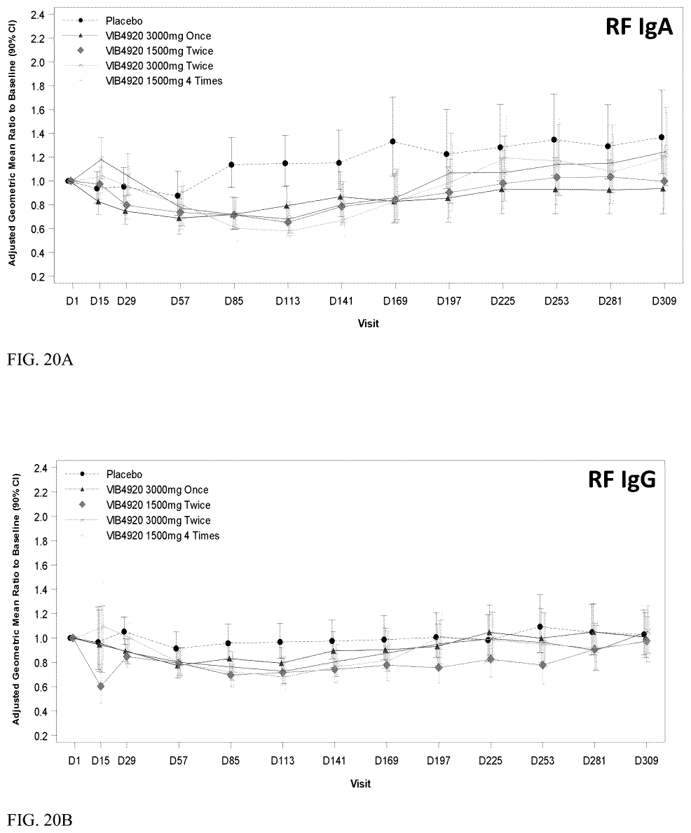

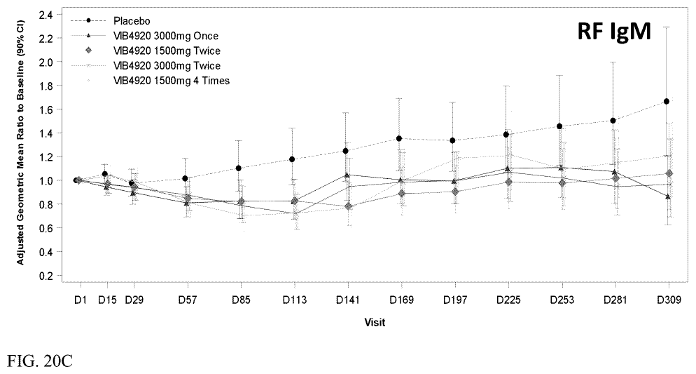

A statistically significant difference in reduction from baseline in RF (FIG. 19) was observed across all 4 VIB4920 doses (VIB4920 3000 mg×1, VIB4920 1500 mg×2, VIB4920 3000 mg×2, and VIB4920 1500 mg×4) compared to placebo at Day 113. VIB4920 1500 mg 4 times group achieved the best reduction.

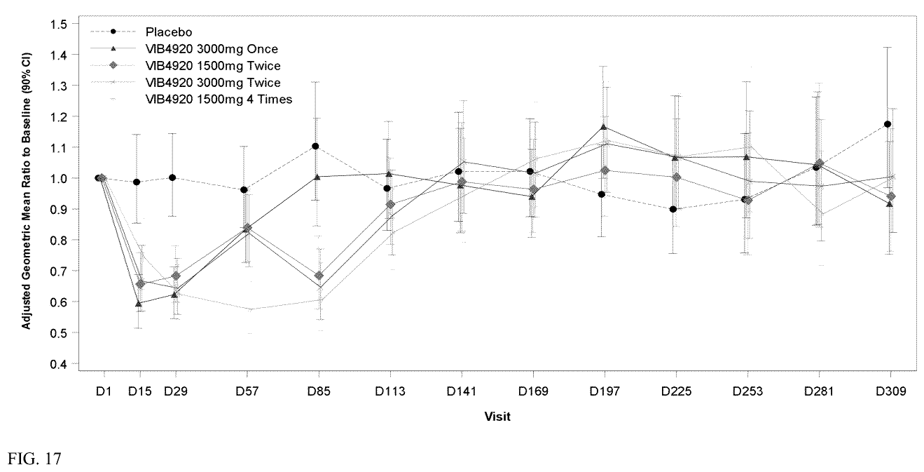

A statistically significant reduction from baseline (˜30-40%) in CXCL13 was observed across all 4 VIB4920 doses (VIB4920 3000 mg×1, VIB4920 1500 mg×2, VIB4920 3000 mg×2, and VIB4920 1500 mg×4) compared to placebo through Day 29, but all but the 1500 mg 4 times group lost suppression by Day 57, with decline achieving statistical significance again observed on Day 85 in all but the single 3000 mg dose group (FIG. 17).

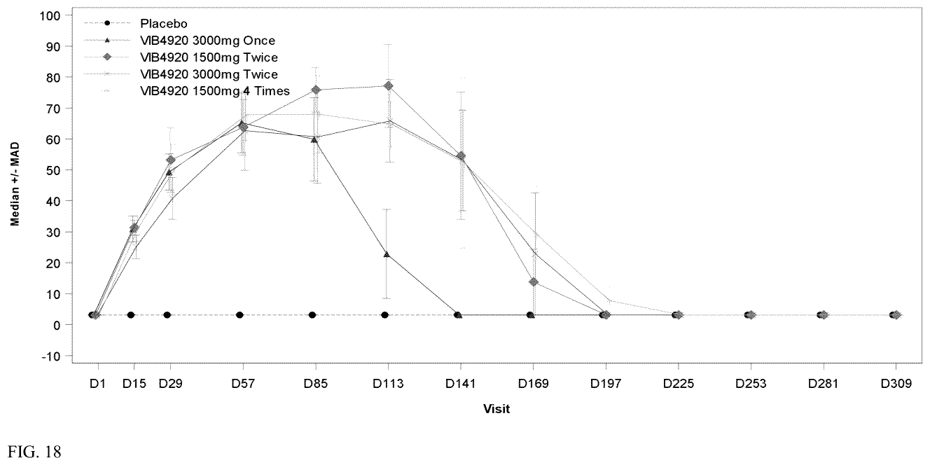

An increase in total sCD40L was observed, indicating binding of VIB4920 to sCD40L and target engagement. Through Day 85, this increase in total sCD40L was not dose-dependent, but by Day 113, the single 3000 mg dosing cohort was substantially different from the other cohorts but still above baseline (FIG. 18).

### Outcome Measures

Table 14 shows baseline measure of disease activity score 28° C.-reactive protein. The DAS28-CRP is a composite index used to assess rheumatoid arthritis disease activity, calculated based on the tender joint count (out of 28 evaluated joints), swollen joint count (out of 28 evaluated joints), Patient's Global Assessment of Disease Activity (0-100 mm), and high-sensitivity C-reactive protein (hsCRP; in mg/L). Scores on the DAS28-CRP range from 0 to approximately 10, where higher scores indicate more disease activity.

TABLE 14
Baseline Measure of Disease Activity Score 28 C-reactive
protein (DAS28-CRP)

| | | VIB4920 | VIB4920 | VIB4920 | VIB4920 | |
|---|---|---|---|---|---|---|
| | | 3000 mg | 1500 mg | 3000 mg | 1500 mg | |
| | Placebo | Once | Twice | Twice | 4 Times | Total |
| Overall # | 16 | 16 | 16 | 15 | 15 | 78 |
| of subjects analysed | | | | | | |
| Mean (SD) | 5.443 (1.066) | 5.473 | 5.945 | 5.452 | 5.761 | 5.615 |
| | | (0.883) | (0.736) | (0.644) | (0.711) | (0.830) |

The total soluble cluster of differentiation 40 ligand (sCD40L) plasma concentration was evaluated over time. Total sCD40L (free sCD40L and sCD40L bound to VIB4920) was measured in plasma samples using a modified commercially available kit. Measurements were taken at Day 1 (Baseline), Days 15, 29, 57, 85,113,141, 169, 197, 225, 253, 281, and day 309. Results are shown in Table 15.

TABLE 15
Total Soluble Cluster of Differentiation 40 Ligand (sCD40L)Plasma
Concentration over time

| | | | VIB4920 | VIB4920 | VIB4920 | VIB4920 |
|---|---|---|---|---|---|---|
| | | | 3000 mg | 1500 mg | 3000 mg | 1500 mg |
| | | Placebo | Once | Twice | Twice | 4 Times |
| Overall Number of Participants | | 16 | 16 | 16 | 15 | 15 |
| Analyzed | | | | | | |
| sCD40LPlasma | [Not | | | | | |
| Concentration | specified] | | | | | |
| Over Time | | | | | | |
| Mean (Standard | | | | | | |
| Deviation) | | | | | | |
| Unit of measure: | | | | | | |
| ng/ml | | | | | | |
| Change at Day 15 | Number | 16 | 16 | 16 | 13 | 14 |
| | Analyzed | participants | participants | participants | participants | participants |
| | | 4.6759 | 30.7744 | 36.0088 | 28.2492 | 33.9007 |
| | | (3.6596) | (7.4954) | (14.4324) | (10.4252) | (8.7777) |
| Change at Day 29 | Number | 16 | 15 | 15 | 14 | 14 |
| | Analyzed | participants | participants | participants | participants | participants |
| | | 4.8431 | 67.5427 | 66.7047 | 68.0636 | 68.1386 |
| | | (3.3770) | (16.4322) | (15.3708) | (28.6523) | (12.1314) |
| Change at Day 57 | Number | 16 | 15 | 15 | 14 | 14 |
| | Analyzed | participants | participants | participants | participants | participants |
| | | 0.8150 | 64.4177 | 62.5560 | 63.6939 | 64.1329 |
| | | (1.6053) | (16.4322) | (14.4445) | (25.5500) | (12.4842) |
| Change at Day 85 | Number | 15 | 15 | 15 | 15 | 14 |
| | Analyzed | participants | participants | participants | participants | participants |
| | | 0.4483 | 53.1583 | 77.5360 | 57.2587 | 69.8821 |
| | | (1.1842) | (20.6513) | (18.7440) | (20.0742) | (19.2623) |
| Change at Day | Number | 15 | 14 | 15 | 14 | 14 |
| | Analyzed | participants | participants | participants | participants | participants |
| 113 | | 0.3420 | 21.4686 | 75.4407 | 53.4514 | 68.5636 |
| | | (1.3246) | (19.3705) | (18.2921) | (26.2712) | (19.3458) |
| Change at Day | Number | 15 | 15 | 14 | 15 | 14 |
| 141 | Analyzed | participants | participants | participants | participants | participants |
| | | 0.2547 | 1.6517 | 55.4204 | 53.2827 | 52.3943 |
| | | (0.9863) | (3.7565) | (28.9470) | (31.6165) | (29.4422) |
| Change at Day | Number | 14 | 15 | 14 | 15 | 13 |
| 169 | Analyzed | participants | participants | participants | participants | participants |
| | | 0.3114 | 0 | 21.1296 | 18.3170 | 23.5727 |
| | | (1.1653) | (0) | (27.1973) | (20.2842) | (26.0510) |
| Change at Day | Number | 14 | 14 | 14 | 15 | 14 |
| | Analyzed | participants | participants | participants | participants | participants |
| 197 | | 0.2757 | 0 | 4.4354 | 2.5057 | 4.6343 |
| | | (1.0316) | (0) | (9.9146) | (4.4880) | (5.3603) |
| Change at Day | Number | 14 | 14 | 14 | 15 | 14 |
| 225 | Analyzed | participants | participants | participants | participants | participants |
| | | 0.2450 | 0 | −0.4043 | −0.0137 | −0.2971 |
| | | (0.9167) | (0) | (1.5127) | (1.1940) | (0.8718) |
| Change at Day | Number | 14 | 14 | 13 | 13 | 13 |
| 253 | Analyzed | participants | participants | participants | participants | participants |
| | | 0.3436 | 3.1418 | −0.5946 | −0.5292 | −0.9485 |
| | | (1.2855) | (11.7555) | (2.1439) | (1.9082) | (2.3281) |
| Change at Day | Number | 14 | 13 | 10 | 14 | 13 |
| 281 | Analyzed | participants | participants | participants | participants | participants |
| | | −0.0343 | 0 | −0.5640 | −0.4729 | −0.9485 |
| | | (0.1283) | (0) | (1.7835) | (1.7693) | (2.3281) |
| Change at Day | Number | 14 | 12 | 10 | 12 | 13 |
| 309 | Analyzed | participants | participants | participants | participants | participants |
| | | −0.5654 | 0 | −0.5720 | −0.7833 | −0.9485 |
| | | (2.1154) | (0) | (1.8088) | (2.7135) | (2.3281) |

The percentage of subjects with positive anti-drug antibodies (ADA) was also evaluated according to the below metrics:
a. ADA was positive at any time: observed at least once during the study (baseline included). Treatment-emergent ADA: ADA positive post-baseline only or boosted pre-existing ADA during the study period.
b. Persistent positive: treatment-induced ADA positive at≥2 post-baseline assessments (with≥16 weeks between first and last positive) or positive at last post-baseline assessment.
c. Transient positive: treatment-induced ADA post-baseline positive but does not fulfill the criteria of persistent positive.

Measurements were taken at Day 1 (Baseline) to Day 309 Day 1 (Baseline) up to Day 309 (±7 days). Results are shown in Table 16.

TABLE 16
Anti-drug antibodies (ADA) Summary

| | VIB4920 3000 | VIB4920 1500 | VIB4920 3000 | VIB4920 1500 |
|---|---|---|---|---|
| | mg Once | mg Twice | mg Twice | mg 4 Times |
| Number of Subjects | 18 | 17 | 13 | 14 |
| ADA positive at any | 44.4 | 11.8 | 38.5 | 28.6 |
| time | | | | |
| Baseline ADA | 0 | 0 | 0 | 0 |
| positive | | | | |
| Baseline only ADA | 0 | 0 | 0 | 0 |
| positive | | | | |
| Post-baseline ADA | 44.4 | 11.8 | 38.5 | 28.6 |
| positive | | | | |
| Treatment-emergent | 44.4 | 11.8 | 38.5 | 28.6 |
| ADA | | | | |
| Persistent Positive | 11.1 | 0 | 0 | 0 |
| Transient Positive | 33.3 | 11.8 | 38.5 | 28.6 |

Change from baseline to Day 113 in Anti-Citrullinated Protein Antibodies (ACPAs) was also evaluated. Excluding data after rescue. Adjusted geometric mean ratio to baseline (90% CI) results are from MMRM analysis on log(ratio to baseline) with treatment, visit, visit by treatment interaction, and log(baseline) included in the model. Ratios less than 1 indicate a decrease. Results are shown in Table 17.

TABLE 17
Change from Baseline to Day 113 in Anti-Citrullinated Protein Antibodies (ACPAs).

| | | VIB4920 3000 | VIB4920 1500 | VIB4920 3000 | VIB4920 1500 |
|---|---|---|---|---|---|
| | Placebo | mg Once | mg Twice | mg Twice | mg 4 Times |
| Number of | 15 | 14 | 15 | 14 | 14 |
| subjects | | | | | |
| analysed | | | | | |
| | 1.08 (0.83 | 0.69 (0.52 | 0.82 (0.62 | 0.84 (0.63 | 0.62 (0.47 |
| | to 1.42) | to 0.91) | to 1.07) | to 1.11) | to 0.82) |

Geometric Mean (90% Confidence Interval). Unit of measure: ratio

Change From Baseline to Day 113 in Rheumatoid Factor (RE) was also evaluated. Excluding data after rescue. Adjusted geometric mean ratio to baseline (90% CI) results are from MMRM analysis on log(ratio to baseline) with treatment, visit, visit by treatment interaction, and log(baseline) included in the model. Ratios less than 1 indicate a decrease. Results are shown in Table 18.

TABLE 18
Change From Baseline to Day 113 in Rheumatoid Factor (RF)

| | | VIB4920 3000 | VIB4920 1500 | VIB4920 3000 | VIB4920 1500 |
|---|---|---|---|---|---|
| | Placebo | mg Once | mg Twice | mg Twice | mg 4 Times |
| Number of | 15 | 14 | 15 | 14 | 14 |
| subjects | | | | | |
| analysed | | | | | |
| | 1.20 (1.04 | 0.77 (0.66 | 0.74 (0.64 | 0.72 (0.62 | 0.57 (0.49 |
| | to 1.39) | to 0.89) | to 0.86) | to 0.84) | to 0.66) |

Geometric Mean (90% Confidence Interval). Unit of measure: ratio

The percentage of subjects with clinical remission at Day 113 was determined. Clinical remission is defined as DAS28-CRP≤2.6. The DAS28-CRP is a composite index used to assess rheumatoid arthritis disease activity, calculated based on the tender joint count (out of 28 evaluated joints), swollen joint count (out of 28 evaluated joints), Patient's Global Assessment of Disease Activity (0-100 mm), and high-sensitivity C-reactive protein (hsCRP; in mg/L). Scores on the DAS28-CRP range from 0 to approximately 10, where higher scores indicate more disease activity. Results are shown in Table 19.

TABLE 19
Percentage of Participants with Clinical Remission at Day 113

| | | VIB4920 3000 | VIB4920 1500 | VIB4920 3000 | VIB4920 1500 |
|---|---|---|---|---|---|
| | Placebo | mg Once | mg Twice | mg Twice | mg 4 Times |
| Number of | 15 | 14 | 15 | 14 | 14 |
| subjects | | | | | |
| analysed | | | | | |
| | 20.0 | 21.4 | 6.7 | 14.3 | 14.3 |

### CONCLUSION

Dazodalibep (VIB4920) reduced DAS28-CRP and RF significantly as compared to placebo at day 113 in all dose regimens tested. The study met its primary endpoint of change from baseline in DAS28-CRP at Day 113 in all four Dazodalibep dosing arms. This endpoint is a standardized measure that is used in RA clinical trials to measure disease activity. Dazodalibep was well tolerated. The Dazodalibep Phase 2 trial follows the Phase 1b, multiple ascending dose study in patients with active moderate-to-severe RA. In this trial, the last dose of Dazodalibep was given at Day 85 and follow-up data at Day 169 showed a prolonged and sustained benefit on disease activity. Of note was that the single 3000 mg administration was as effective as multiple administrations of Dazodalibep, thereby indicating the viability of less frequent dosing. Indeed, treatment effects were observed at day 113 and the prolonged duration of responses support less frequent dosing.

### Example 2—Combining a CD40L-Binding Protein (VIB4920) with a TNF-α Inhibitor for the Treatment of Inadequately Controlled Rheumatoid Arthritis: CD40L antagonism in RA

### Study Design

A phase 2, multi-site, prospective, randomized, placebo-controlled, three-arm [two arms double-blinded, one arm evaluator-blinded (subject is aware of his/her treatment status, but evaluator is not)] trial of VIB4920, an exemplary Tn3 scaffold, in 104 adults with seropositive RA in the United States is described. Subjects are eligible if they have moderate or high disease activity (Simplified Disease Activity Index [SDAI]>17) despite treatment with a TNFi (etanercept or adalimumab) for at least 12 weeks. The schedule of events for the study drug administration period is provided in Table 22.

Subjects are Randomized in a 2:1:1 Fashion into One of the Following Three Study Arms:
(1) VIB4920 with TNFi (n≈52): Subjects will receive VIB4920 in a blinded fashion intravenously at a dose of 1500 mg at weeks 0, 2, 4, 8, and 12 and continue all background disease-modifying RA therapy, including the TNFi, through the study period.
(2) VIB4920 placebo with TNFi (n≈26): Subjects will receive VIB4920 placebo in a blinded fashion intravenously at weeks 0, 2, 4, 8, and 12 and continue all background disease-modifying RA therapy, including the TNFi, through the study period.
(3) VIB4920 without TNFi (n≈26): Subjects will stop TNFi after randomization to this arm, and receive VIB4920 in an evaluator-blinded fashion intravenously at a dose of 1500 mg at weeks 0, 2, 4, 8, and 12 while maintaining all other background disease-modifying RA therapy (e.g., methotrexate, hydroxychloroquine, etc.) through the study period. This arm is evaluator blinded (not aware of treatment status), with the subject aware of treatment status due to not using a TNFi placebo this study.

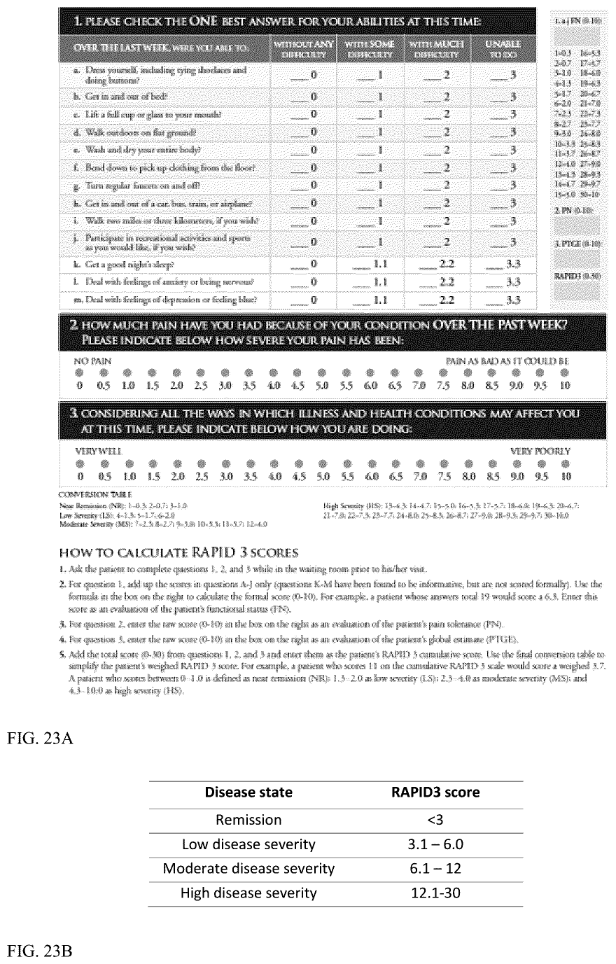

After week 12, all subjects randomized to VIB4920 or VIB4920 placebo will continue treatment with their background disease-modifying drugs, including their TNFi, and are followed through week 40. Subjects randomized to receive VIB4920 after stopping their TNFi will not restart their TNFi and are followed through week 40. In addition, subjects who achieve the primary endpoint at week 16 are followed through week 40 and undergo weekly home fingerstick blood sampling (“dense sampling”) and RAPID3 assessment, see FIG. 23A and FIG. 23B, to explore transcriptional signatures associated with any increases in disease activity after achieving a pre-defined treatment response (SDAI<11 at week 16).

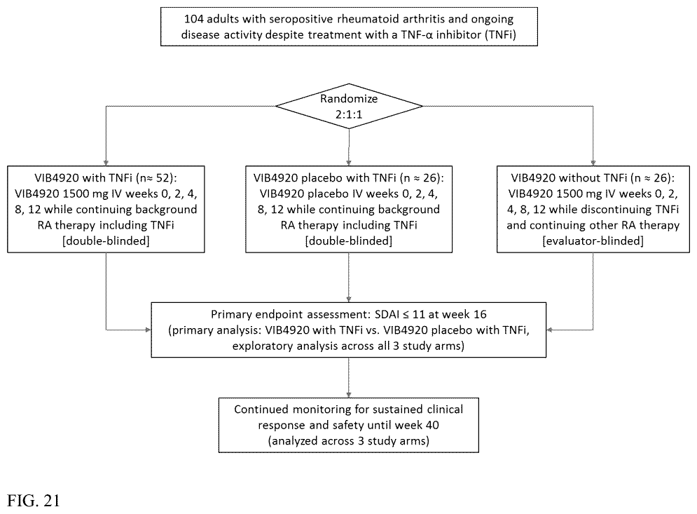

Three arms are included in this study to assess the efficacy of adding VIB4920 to background disease modifying RA therapy including TNFi and replacing TNFi with VIB4920, as well as the safety of this combination of biologic agents compared to either agent alone. Subjects are assessed for the primary endpoint, achievement of low disease activity (defined as SDAI≤11), at week 16. Subjects will then be followed until week 40 while they continue their other disease-modifying treatments to assess for sustained clinical response and safety. Subjects who achieve the primary endpoint at week 16 are eligible to undergo weekly blood sampling and RAPID3 evaluation at home. This weekly blood sampling is optional for eligible subjects and are obtained from week 16 through week 40 using a fingerstick method. This “dense sampling” approach is used to explore transcriptional signatures that may be associated with increases in disease activity, see schedule of events in Table 23. Subjects who do not achieve the primary endpoint or elect not to participate in the optional dense sampling collection are followed from week 16 to week 40 using the schedule of events provided in Table 24. All clinical assessments are performed by a blinded study assessor for the entire duration of the study, see FIG. 21.

### Endpoints

The primary endpoint is the proportion of subjects achieving low disease activity, as defined by a SDAI≤11 at week 16. In addition, subjects who take prohibited medications as treatment for RA prior to week 16 are considered to have failed the primary endpoint. The primary analysis of the primary endpoint is performed on the mITT sample utilizing subjects in the VIB4920 with TNFi and VIB4920 placebo with TNFi arms and is designed to test the following hypotheses:

Null hypothesis: The proportion achieving low disease activity at week 16 does not differ between the VIB4920 with TNFi and VIB4920 placebo with TNFi groups.

Alternate hypothesis: The proportion achieving a low disease activity at week 16 differs between the VIB4920 with TNFi and VIB4920 placebo with TNFi groups.

The proportion of mITT subjects achieving low disease activity at week 16 are estimated for the VIB4920 with TNFi and VIB4920 placebo with TNFi groups. Groups are compared using a two-sided Fisher's Exact test evaluated using a Type 1 error rate of α=0.10.

All secondary endpoints are assessed across all three study treatment arms.

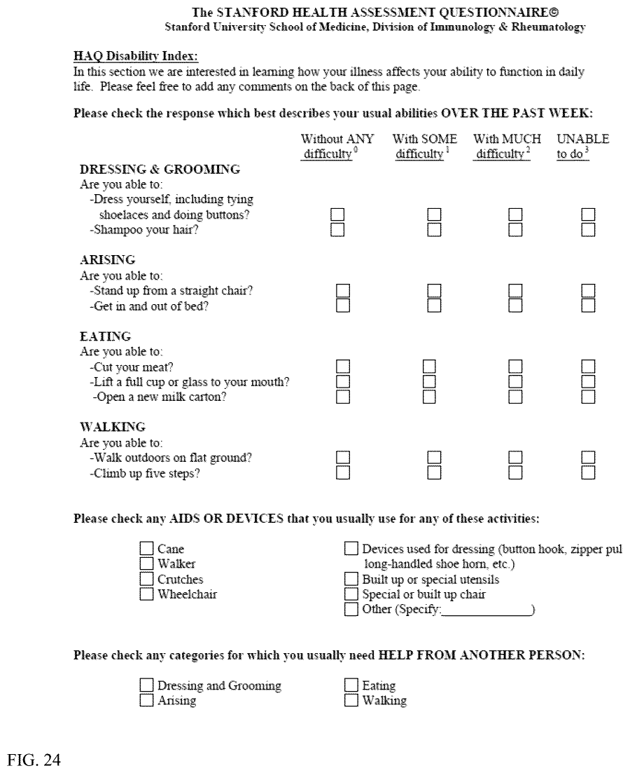

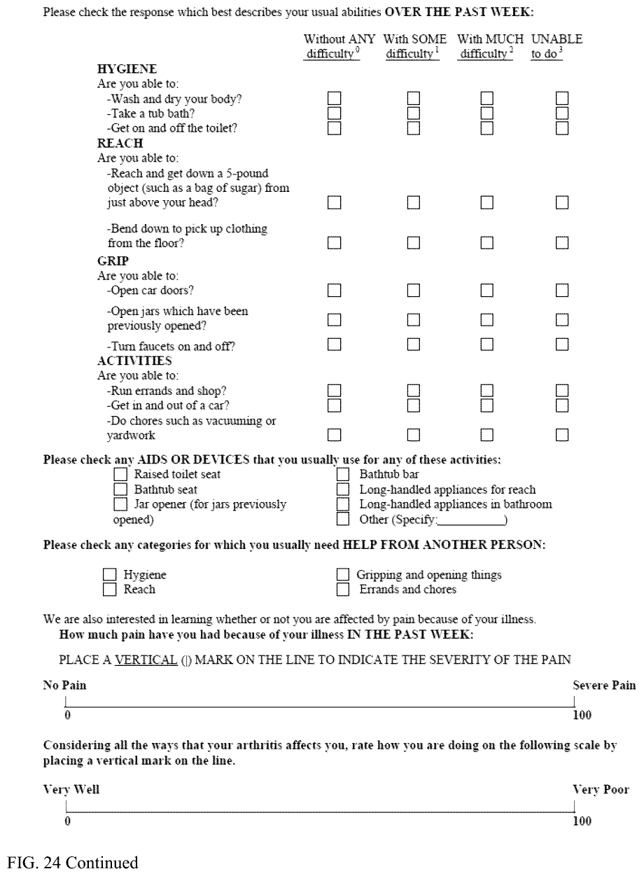

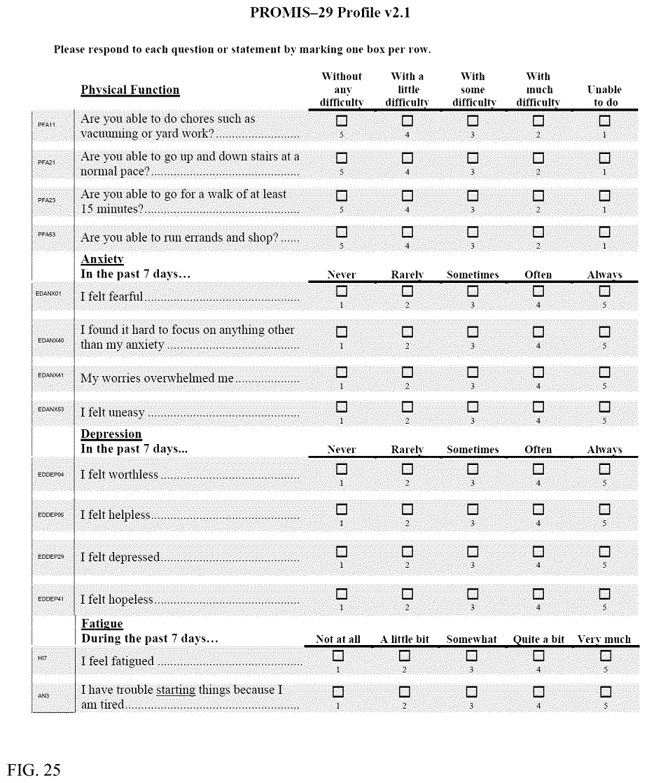

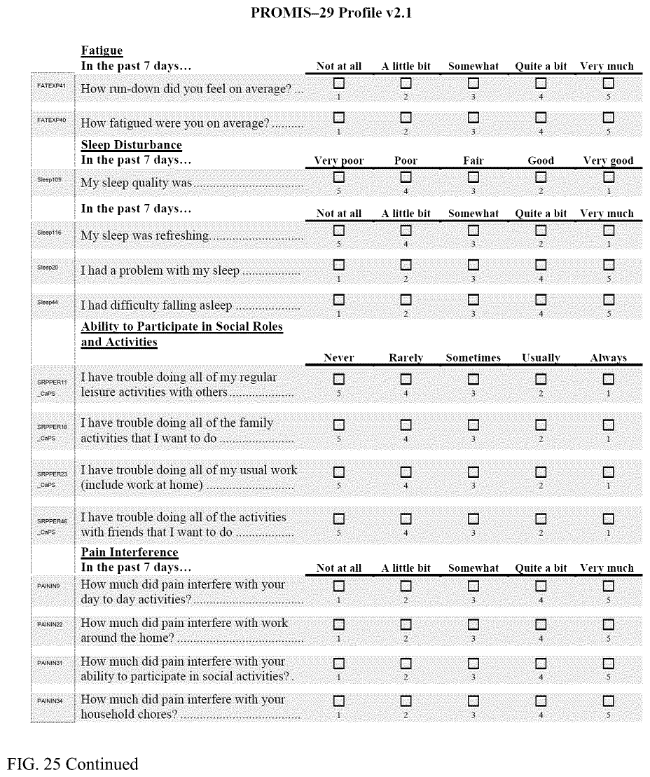

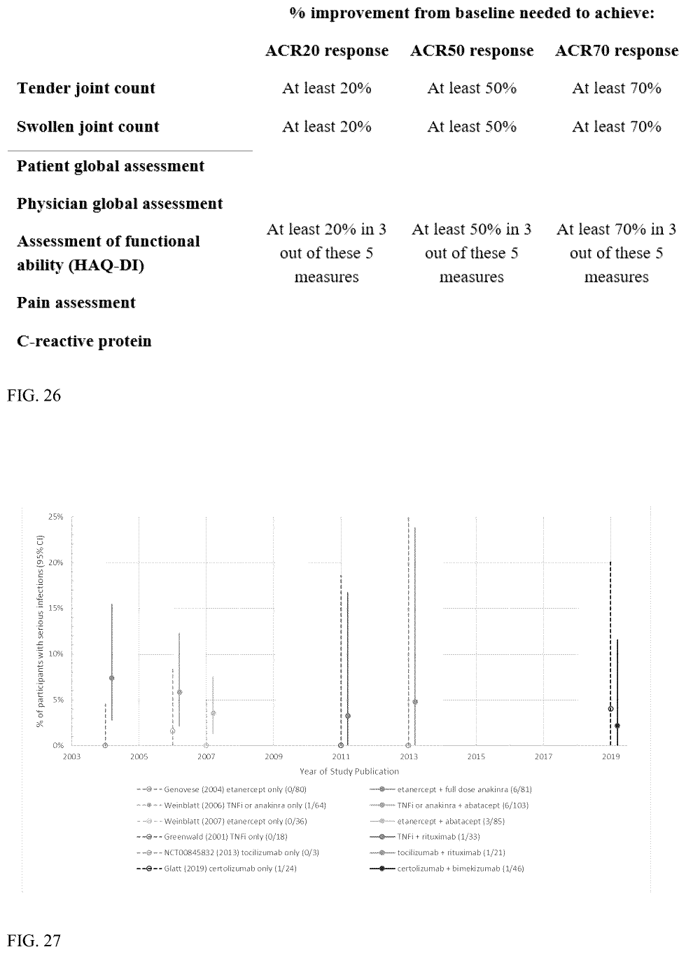

Secondary efficacy endpoints: (A) Sustained remission. (1) Proportion of subjects who achieve sustained remission defined by SDAI≤3.3 at all available disease activity assessments between week 16 and week 40. (B) Low disease activity by DAS28-CRP. (2) Proportion of subjects achieving low disease activity defined by DAS28-CRP≤3.2 at week 16. (C) Remission. (3) Proportion of subjects achieving remission defined by SDAI≤3.3 at week 16. (4) Proportion of subjects achieving remission defined by DAS28-CRP≤2.6 at week 16. (D) ACR20/50/70 endpoints. (5) The proportion of subjects achieving an ACR20 response at week 16. (6) The proportion of subjects achieving an ACR50 response at week 16. (7) The proportion of subjects achieving an ACR 70 response at week 16. (8) The proportion of subjects achieving an ACR20 response at week 40. (9) The proportion of subjects achieving an ACR50 response at week 40. (10) The proportion of subjects achieving an ACR 70 response at week 40. (E) Time to low disease activity or remission. For subjects who fail to achieve low disease activity or remission prior to escalating their disease-modifying therapy or taking a prohibited medication for treatment of RA, low disease activity is assumed and remission is not achievable within 40 weeks. (11) Time to first occurrence of low disease activity as defined by SDAI≤11. (12) Time to first occurrence of low disease activity as defined by DAS28-CRP≤3.2. (13) Time to first occurrence of remission as defined by SDAI≤3.3. (14) Time to first occurrence of remission as defined by DAS28-CRP≤2.6. (F) Time to loss of low disease activity or remission. Subjects who escalate their disease-modifying therapy or take prohibited medications for treatment of their RA are considered to have lost the low disease activity or remission response. (15) Time to loss of low disease activity defined by SDAI>11 for the subset of subjects achieving low disease activity by the SDAI criteria at week 16. (16) Time to loss of low disease activity defined by DAS28-CRP>3.2 for the subset of subjects achieving low disease activity by the DAS28-CRP criteria at week 16. (17) Time to loss of remission defined by SDAI>3.3 for the subset of subjects achieving remission by the SDAI criteria at week 16. (18) Time to loss of remission defined by DAS28-CRP>2.6 for the subset of subjects achieving remission by the DAS28-CRP criteria at week 16. (E) Longitudinal trends. (19) Longitudinal trends in SDAI from week 0 to week 40. (20) Longitudinal trends in DAS28-CRP from week 0 to week 40. (H) HAQ-DI and PROMIS. (21) Change in the Health Assessment Questionnaire—Disability Index (HAQ-DI), see FIG. 24, from week 0 to week 16. (22) Change in PROMIS-29 profile scores, see FIG. 25, from week 0 to week 40. (23) Change in HAQ-DI from week 0 to week 40. (24) Change in PROMIS-29 from week 0 to week 16. 03331 Secondary safety endpoints: (a) Incidence of grade 2 or higher adverse events; (b) Incidence of serious adverse events; and (c) Incidence of adverse events of special interest.

### Population

The study recruits subjects with inclusion and exclusion criteria as in Table 20. The study recruits subjects with seropositive RA who have had an inadequate response to a TNFi. Subjects with persistent disease activity despite treatment with a TNFi (with or without methotrexate or other cDMARD) are at risk for progressive joint damage and are candidates fora change in disease-modifying therapy. Subjects with active disease that are receiving a TNFi are enrolled as it is hypothesized that combining TNF-ca inhibition with an agent that interferes with a dysregulated adaptive immune response, in this case a drug targeting the CD40L-CD40 pathway, will improve disease control and lead to a sustained clinical benefit. The study will enroll adults that are 70 years of age or younger to mitigate the impact of infections, which may be a risk for subjects receiving this untested combination of biologic agents. In addition, subjects with clinical or laboratory features associated with an increased risk for infection will not be eligible for this trial. Subjects will need to have moderate or high disease activity, as well as a sufficient number of tender and swollen joints that would warrant a change in treatment strategy.

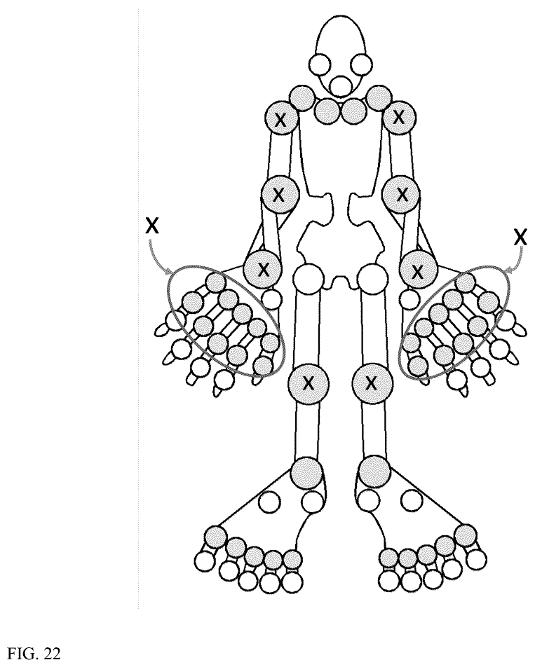

TABLE 20
Inclusion and Exclusion Criteria

| Inclusion | Subjects who meet all of the following criteria at screening are eligible for enrollment: | |
|---|---|---|
| Criteria | 1. | Subject or legally authorized representative must be able to understand and provide |
| | | informed consent |
| | 2. | Adult 18-70 years of age |
| | 3. | Diagnosed with RA by fulfilling the ACR/EULAR 2010 Classification Criteria for |
| | | RA ≥6 months prior to screening |
| | 4. | Documented positive test for rheumatoid factor (RF) and/or anti-cyclic citrullinated |
| | | peptide antibody (ACPA) |
| | 5. | SDAI ≥17 |
| | 6. | At least 4 tender and 4 swollen joints by a 44 joint count (FIG. 22) |
| | 7. | TNFi therapy: |

| | |
|---|---|
| a. | Current treatment with etanercept 50 mg SC weekly or adalimumab 40 mg SC |
| | every other week for at least 12 weeks |
| b. | Willing to continue treatment with their current TNFi at the same dose or to |
| | discontinue it depending upon study arm assignment. |

| | 8. | If treated with leflunomide, sulfasalazine, or hydroxychloroquine, must be taking a |
|---|---|---|
| | | stable dose for at least 12 weeks. |
| | 9. | If treated with methotrexate, must be taking a stable dose for at least 12 weeks. The |
| | | following exceptions are permitted within the 12 weeks prior to screening: |
| | | Holding methotrexate after SARS-CoV-2 vaccination as per American College of |
| | | Rheumatology guidance |
| | | Holding methotrexate for 1 or 2 weeks after influenza vaccination |
| | 10. COVID-19 vaccination: | |
| | | Completion of a primary COVID-19 vaccination series based on current CDC |
| | | recommendations for individuals who are moderately to severely |
| | | immunocompromised. The primary vaccination series should include at least 2 doses |
| | | of an mRNA vaccine, one dose of an adenovirus-based vaccine, or the primary series |
| | | for any other authorized or approved vaccine. |
| | | Receipt of at least one booster dose of a COVID-19 vaccine after the primary vaccine |
| | | series if recommended by the CDC for individuals who are moderately to severely |
| | | immunocompromised |
| | | The last COVID-19 vaccine dose must have been administered at least 14 days |
| | | prior the initiation of the study drug (Visit 0) |
| | | All subjects who engage in sexual activity that could lead to pregnancy must agree |
| | to use abstinence or an FDA-approved contraception for the duration of the study to prevent | |
| | pregnancy. | |
| Exclusion | Subjects who meet any of these criteria at screening are not eligible for enrollment as study | |
| Criteria | subjects: | |
| | 1. | Inability or unwillingness to give written informed consent or comply with the study |
| | | protocol |
| | 2. | Prior or ongoing systemic inflammatory or autoimmune disease (other than RA and |
| | | secondary Sjögren's syndrome) requiring or potentially requiring other systemic |
| | | immunomodulatory therapy during the 40-week study period |
| | 3. | Use of glucocorticoid and/or disease-modifying therapies as specified below: |

| | |
|---|---|
| a. | Prior treatment with any B cell depleting therapy (e.g., rituximab) |
| b. | History of treatment with more than two TNFi, including ongoing treatment with |
| | etanercept or adalimumab |
| c. | Treatment with other biologic therapy (i.e., not targeting TNF-α), including |
| | abatacept, tocilizumab, or sarilumab within the previous 12 weeks |
| d. | Treatment with a JAK inhibitor within the previous 12 weeks |
| e. | Concurrent use of methotrexate and leflunomide |
| f. | Prednisone >10 mg a day or equivalent glucocorticoid use within the previous 4 |
| | weeks |
| g. | Intramuscular, intra-articular, or intravenous glucocorticoids within the previous |
| | 4 weeks |
| h. | Other immunomodulatory medications within the previous 12 weeks except for |
| | methotrexate, leflunomide, sulfasalazine, or hydroxychloroquine |

| | |
|---|---|
| 4. | Lack of any subjective or objective clinical response (i.e., complete non-responder) |
| | to current TNFi use, in the opinion of the study investigator based on information |
| | provided by the subject and referring rheumatologist |
| 5. | Use of an investigational agent including VIB4920 in the past 30 days or 5 half- |
| | lives, whichever is longer |
| 6. | History of a severe allergy, hypersensitivity reaction, or infusion reaction to any |
| | component of the VIB4920 formulation |
| 7. | History of Felty's syndrome |
| 8. | History of interstitial lung disease with FVC <70% predicted, DLCO <70% |
| | predicted, or requiring supplemental oxygen |
| 9. | Hypercoagulable state as specified below: |

| | |
|---|---|
| a. | Previous deep venous or arterial thrombosis or thromboembolism, or pulmonary |
| | embolism |
| b. | Known hypercoagulable state (e.g., inherited thrombin III deficiency, protein S |
| | deficiency, protein C deficiency, antiphospholipid antibody syndrome, MTHFR |
| | mutation) |
| c. | Risk factors for deep venous or arterial thromboembolism (e.g., immobilization |
| | or major surgery within 12 weeks prior to enrolment) |
| d. | Anti-phospholipid antibodies: |

| | |
|---|---|
| i. | Positive anti-cardiolipin IgG, IgM, or IgA antibodies at a moderate titer or |
| | higher (≥40 U) |
| ii. | Positive anti-beta-2-glycoprotein I IgG, IgM, or IgA antibodies at a |
| | moderate titer or higher (≥40 U) |
| iii. | Positive lupus anticoagulant test |

| | |
|---|---|
| 10. | Infection: |

| | |
|---|---|
| a. | Evidence of current or prior infection with hepatitis B, as indicated by a positive |
| | test for the hepatitis B surface antigen (HBsAg) or a positive test for the hepatitis |
| | B core antibody (HBcAb) |
| b. | Positive HCV serology unless treated with an anti-viral regimen resulting in a |
| | sustained virologic response (undetectable viral load 24 weeks after cessation of |
| | therapy) |
| c. | Evidence of HIV infection |
| d. | Evidence of active tuberculosis, untreated or incompletely treated latent |
| | tuberculosis, or recent close contact with a person who has active tuberculosis |
| e. | Positive QuantiFERON-TB Gold, QuantiFERON-TB Gold Plus, or T-SPOT.TB |
| | test without history of completing treatment for active or latent TB |
| f. | Indeterminate QuantiFERON-TB Gold, QuantiFERON-TB Gold Plus, or T- |
| | SPOT.TB test which remains indeterminate on repeat testing, and any of the |
| | following additional required screening which indicates an increased risk of TB |
| | infection: |

History of tuberculosis exposure
History of travel to an area where tuberculosis is endemic
Findings on chest radiograph suggestive of prior exposure to tuberculosis
(e.g., granulomas or apical scarring) obtained at screening or within the past
3 months
Positive purified protein derivative (PPD) skin test for tuberculosis, either
obtained at screening or within in the past 3 months
Prior history of a positive QuantiFERON-TB Gold, QuantiFERON-TB
Gold Plus, T-SPOT.TB, or purified protein derivative (PPD) test without
history of previous treatment for latent TB

| | |
|---|---|
| g. | Positive test for acute COVID-19 infection (e.g., PCR test for SARS-CoV-2 or |
| | alternative viral test according to CDC guidance) |
| h. | Symptoms of presumed or documented COVID-19 infection in the past 30 days |
| i. | More than one episode of herpes zoster in the past 12 months |
| j. | An opportunistic infection in the past 12 months |
| k. | Acute or chronic infection, including current use of suppressive systemic anti- |
| | microbial therapy for chronic or recurrent bacterial or fungal infection, |
| | hospitalization for treatment of infection in the past 60 days, or parenteral anti- |
| | microbial (including anti-bacterial, anti-viral, or anti-fungal agents) use in the |
| | past 60 days for infection |
| l. | History of bronchiectasis with recurrent pulmonary infections |

| | |
|---|---|
| 11. | History of a primary immunodeficiency disorder |
| 12. | Vaccination with a live vaccine within the past 30 days |
| 13. | Women who are pregnant or breast-feeding |
| 14. | WBC count <3.0 × 10³/μl |
| 15. | Absolute neutrophil count <1.5 × 10³/μl |
| 16. | Hemoglobin <9 g/dL |
| 17. | Platelet count <100 × 10³/μl |
| 18. | Alanine aminotransferase (ALT) or aspartate aminotransferase (AST) ≥2x the upper |
| | limit of normal (ULN) |
| 19. | History of malignant neoplasm within the last 5 years, except for basal cell or |
| | squamous cell carcinoma of the skin treated with local resection only or carcinoma |
| | in situ of the uterine cervix treated locally |
| 20. | Current, diagnosed, mental illness or current, diagnosed or self-reported drug or |
| | alcohol abuse that, in the opinion of the investigator, would interfere with the |
| | subject's ability to comply with study requirements |
| 21. | Any new or uncontrolled condition occurring within the past 12 weeks which, in the |
| | judgment of the investigator, could interfere with participation in the trial (e.g., |
| | diabetes mellitus with HbA1c ≥9.0%, myocardial infarction, or stroke) |
| 22. | Past or current medical problems or findings from physical examination or |
| | laboratory testing that are not listed above, which, in the opinion of the investigator, |
| | may pose additional risks from participation in the study, may interfere with the |
| | subject's ability to comply with study requirements, or that may impact the quality or |
| | interpretation of the data obtained from the study |
| 23. | Inability to comply with study and follow-up procedures. |

### Treatment

Subjects will receive VIB4920 or VIB4920 placebo given intravenously at a dose of 1500 mg at weeks 0, 2, 4, 8, and 12. Subjects randomized to VIB4920 with TNFi or VIB4920 placebo with TNFi will continue their background disease-modifying therapy, including their TNFi. Subjects randomized to VIB4920 without TNFi will discontinue TNFi therapy while maintaining all other background disease-modifying RA therapy.

### Dose Formulation

VIB4920 is administered at a dose of 1500 mg as an intravenous infusion at weeks 0, 2, 4, 8, and 12. To prepare the dose, VIB4920 is removed from refrigerated storage conditions and allowed to equilibrate in the carton for 15 minutes to 2 hours. The investigational product is diluted into 250 mL 0.9% saline for IV infusion. The diluted investigational product can be stored for a maximum of 24 hours at 2-8° C. or 4 hours at room temperature, including preparation time. The investigational product is prepared by an unblinded pharmacist and covered by an opaque bag before being given to blinded study personnel for administration.

VIB4920 placebo is administered as an intravenous infusion at weeks 0, 2, 4, 8, and 12. For the VIB4920 placebo, a bag of 250 mL 0.9% saline is stored in the same fashion as VIB4920 and covered by an opaque bag before being given to blinded study personnel for administration. Subjects are administered VIB4920 or VIB4920 placebo in a clinical trial facility and monitored for evidence of an infusion reaction. Appropriate drugs and medical equipment to treat acute hypotensive, broncho constrictive, or anaphylactic reactions are immediately available, and study personnel trained to recognize and treat these reactions are available. VIB4920 or VIB4920 placebo are administered over a minimum of 90 minutes. The infusion is slowed down or discontinued if there is evidence of an infusion reaction. Subjects are monitored for 2 hours after the first three doses of study treatment, and then for 1 hour after the subsequent doses of study treatment. Vital signs are monitored prior to the infusion and approximately every 30 minutes during treatment administration and the subsequent observation period. VIB4920 or VIB4920 placebo is not administered on the same days as the TNFi. If VIB4920 or VIB4920 placebo and TNFi are scheduled to be administered on the same day, then the TNFi should be administered the day before or day after VIB4920 or VIB4920 placebo administration and maintained on that treatment schedule.

### Toxicity Management

Infusion or hypersensitivity reactions: Subjects are monitored for infusion or hypersensitivity reactions, and receive VIB4920 or VIB4920 placebo in a clinical trial facility with personnel, medications, and equipment available to treat these types of reactions. Anti-histamine (e.g., diphenhydramine or cetirizine) and/or acetaminophen can be administered per institutional guidelines prior to VIB4920 infusion to help prevent infusion reactions. Administration of glucocorticoids prior to VIB4920 infusion to help prevent infusion reactions is not permitted. Administration of study treatment is permanently discontinued if the subject develops a grade 3 or greater hypersensitivity, anaphylactic, or infusion reaction.

Infection: clinical assessments for signs of infection are performed at each study visit. Subjects are contacted by telephone at study weeks 6, 10, and 14 to assess for infection. Subjects are given instructions on potential signs of infection and instructed to contact the site investigator if they have signs or symptoms of an infection. VIB4920/VIB4920 placebo administration is discontinued if the subject is diagnosed with an active SARS-CoV-2 (COVID-19) infection, confirmed by PCR or alternative viral test according to CDC guidance, independent of symptoms or grade of infection. VIB4920/VIB4920 placebo is suspended if the subject develops a non-COVID-19 infection that is grade 2 or greater where systemic treatment (e.g., antibiotic, antifungal or antiviral) is indicated, or the investigator judges to be significant. If the infection resolves, VIB4920 or placebo may be restarted at the next scheduled dose at the investigator's discretion.

Thromboembolic events: Thromboembolic events are monitored by AE evaluations and physical examinations performed during the study. Study treatment is suspended if a subject is suspected to have a deep venous thrombosis or arterial thrombotic event. Study treatment is permanently discontinued for subjects who have a confirmed deep vein thrombosis or arterial thromboembolic event.

Liver chemistry abnormalities: Subjects are monitored for drug induced liver injury. If liver chemistries are found to be abnormal in a subject, they should be repeated in 1-2 weeks for confirmation. If liver chemistry abnormalities are confirmed, then the dosing of VIB4920 or VIB4920 placebo should be adjusted as shown in Table 21.

TABLE 21
Dose Modification for Liver Chemistry Abnormalities

| ALT/AST | Action |
|---|---|
| <3xULN | Maintain dosing |
| 3-5xULN or 1.5-3x baseline if baseline | Suspend study treatment at first occurrence. |
| abnormal, with serum total bilirubin < | Recheck ALT/AST prior to the next |
| 2xULN | expected dose and if AST and ALT < |
| | 3xULN (<1.5-3x baseline if baseline |
| | abnormal), resume treatment per study |
| | schedule. |
| | Permanently discontinue study treatment if |
| | ALT or AST > 3xULN a second time. |
| >3xULN with serum total bilirubin > | Discontinue study treatment. |
| 2xULN | |
| (Hy's Law), regardless of baseline values | |
| >5xULN, regardless of baseline values | Discontinue study treatment. |

### Alternate Monitoring Schedule

Subjects with persistent disease activity that may require a change in disease-modifying therapy

From week 12 to week 40, a subject may be considered for a change in disease modifying treatment regimen (e.g., change TNFi, increase dose of disease-modifying medication, switch to another disease-modifying drug, increase glucocorticoid use, or use a prohibited disease-modifying medication described herein) if either of the following occurs: (a) The subject has an SDAI>26 at week 12 or later, which is confirmed at a study visit (scheduled or unscheduled) in the next 2-4 weeks; (b) The subject has an SDAI>11 at week 16 or later, and his/her SDAI has decreased by less than 50% from baseline, and both are confirmed at a study visit (scheduled or unscheduled) in the next 2-4 weeks.

Subjects who fulfill any of these criteria above are treated at the discretion of the subject's rheumatologist/treating physician. Alternate monitoring includes assessment of grade 2 or higher adverse events that received medical attention, AESIs, and concomitant medications at weeks 2, 4, 8, 12, 16, 24, 32, and 40, as well as disease activity assessments at weeks 16 and 40, if those visits have not already occurred. More frequent assessments can occur, as clinically indicated.

### Concomitant Medications

### Protocol Mandated

Subjects randomized to the VIB4920 with TNFi or VIB4920 placebo with TNFi arms are to continue their prescribed TNFi (adalimumab or etanercept) and maintain the same dose (the dose at study entry) until week 40 unless the TNFi is changed for persistent or worsening disease activity. Subjects randomized to VIB4920 without TNFi are required to stop TNFi use at randomization (week 0).

For all subjects, changes in disease-modifying therapy may be considered after week 12 in the setting of persistent or worsening disease activity.

### Other Permitted Medications

### DMARDS

The following DMARDs are permitted at study entry. Their dose is maintained at the same dose for the duration of the study unless the dose is reduced or the medication is stopped due to toxicity.
1. Methotrexate: up to 25 mg oral or subcutaneous weekly. Methotrexate may be held for 1 week after SARS-CoV-2 or influenza vaccination.
2. Leflunomide: up to 20 mg oral daily. Subjects cannot receive methotrexate and leflunomide concurrently.
3. Hydroxychloroquine: up to 400 mg oral daily.
4. Sulfasalazine: up to 3 g oral daily

### Glucocorticoids

Oral prednisone is permitted up to 10 mg oral daily (or equivalent dose of other oral glucocorticoid) at study entry. The prednisone dose cannot be tapered prior to the assessment of the primary endpoint at week 16. Prednisone can be tapered between weeks 16 to 30 if the subject is in remission (SDAI≤3.3). Prednisone can be tapered at the investigator's discretion and according to subject preferences, but it is recommended to be not faster than decreasing the daily dose by 2.5 mg every two weeks for subjects taking prednisone 5-10 mg/day, and not faster than 1 mg every 2 weeks for subjects taking prednisone 1-5 mg/day. One course of oral prednisone can be used to treat conditions other than RA, provided the dose does not exceed 40 mg daily and the duration is <2 weeks. This course of prednisone cannot occur within weeks 12-16 or weeks 36-40. One intra-articular or bursa injection of glucocorticoids is permitted after the assessment of the primary endpoint between weeks 16-32, provided the dose does not exceed 40 mg of triamcinolone (or equivalent dose of another injectable glucocorticoid).

### Other Medications

Use of non-steroidal anti-inflammatory medications is permitted. While not prohibited, use of herbal remedies is discouraged and should be discussed with the site investigator. SARS-CoV-2 vaccines approved by the FDA or available under Emergency Use Authorization are considered to be permitted concomitant treatments. Subjects are instructed not to take any new medications or over-the-counter products without first consulting with the site investigator unless the medications are prescribed by a healthcare provider for another medical condition that develops or worsens during the study.

No prophylactic medications are required by this protocol. It is recommended that subjects be up-to-date with their recommended vaccinations at least 2 weeks prior to study entry, since VIB4920 may decrease responses to vaccinations. It is recommended that subjects do not receive non-live vaccines from the start of screening (Visit −1) to the end of study drug treatment (week 16). Completion of SARS-CoV-2 vaccination at least 2 weeks prior to study screening is recommended for all subjects willing and able to receive the vaccine.

### Prohibited Medications and Procedures

The following medications and procedures are prohibited: Any investigational drug or treatment other than VIB4920, Live-attenuated vaccines, Concurrent use of methotrexate and leflunomide, Injection of corticosteroids into joints or bursae, except as permitted after the assessment of the primary endpoint and between weeks 16-32, Intravenous glucocorticoids, unless used to treat infusion reactions, Intramuscular injections of glucocorticoids, Oral prednisone at doses or duration greater than herein described, Addition of a new treatment or increase in dose of current disease modifying therapy to treat RA except as specified herein, Plasmapheresis or plasma exchange, any other medication that fulfills exclusion criteria described herein.

### Study Assessments

### General

(1) Informed consent: Written informed consent is obtained before any study assessments or procedures are performed. (2) Eligibility criteria: Eligibility for study participation is assessed during the screening period. (3) Demographics: age, gender and ethnicity. (4) Medical history: A history is taken to determine if the subject has had any clinically significant diseases or medical procedures other than the disease under study. (5) Rheumatoid arthritis history, including date of diagnosis and previous treatments. (6) Comprehensive physical examination includes body systems: musculoskeletal, respiratory, cardiovascular, gastrointestinal, skin, neurologic and renal/urinary. (7) Limited physical examination focuses on the musculoskeletal exam and body systems relevant to the subject's clinical complaints and clinical status at the study visit. (8) Adverse events: Subjects are assessed for adverse events. All adverse events are graded, recorded on the case report forms (CRFs). (9) Concomitant medications: All concomitant medications and their indications are recorded. (10) Vital signs: Height and weight are obtained at screening (V-1); weight, temperature, blood pressure, respiration, and pulse are obtained at all visits. Temperature, blood pressure, respiration, and pulse are obtained prior to VIB4920/VIB4920 placebo infusion, and approximately every 30 minutes (±5 min) during study drug administration and for 2 hours after completion of the first 3 infusions, and for 1 hour after completion of the remaining infusions.

### Clinical Laboratory Assessments

(1) Hematology: CBC with differential. (2) Chemistry: Creatinine, total bilirubin, AST, ALT, alkaline phosphatase, and albumin. (3) Inflammatory markers: Erythrocyte sedimentation rate, C-reactive protein. (4) HIV (RNA or antibody). (5) Hepatitis B (surface antigen, core antibody). (6) Hepatitis C (RNA or antibody). (7) Tuberculosis testing: QuantiFERON-TB Gold, QuantiFERON-TB Gold Plus, or T-SPOT.TB test. PPD skin test for participants with an indeterminate QuantiFERON-TB Gold, QuantiFERON-TB Gold Plus, or T-SPOT.TB test, if not performed within the past 3 months. AP and lateral chest radiograph for subjects with an indeterminate QuantiFERON-TB Gold, QuantiFERON-TB Gold Plus, or T-SPOT.TB test, if not performed within the past 3 months. (8) Anti-phospholipid antibodies (anti-cardiolipin IgG, IgM, and IgA; anti-beta2-glycoprotein I IgG, IgM, and IgA; lupus anticoagulant). (9) Rheumatoid factor (RF). (10) Anti-citrullinated peptide antibodies (ACPA). (11) Serum pregnancy (for women with childbearing potential). (12) STAT urine pregnancy (for women with childbearing potential). (13) PCR test for SARS-CoV-2 (or alternative viral test according to CDC guidance).

Adverse events are graded on a scale from 1 to 5 according to the following standards in the NCI-CTCAE manual: Grade 1=mild adverse event. Grade 2=moderate adverse event. Grade 3=severe adverse event. Grade 4=life-threatening adverse event or urgent intervention indicated. Grade 5=death.

For this study, liver chemistry abnormalities are graded using protocol specific criteria, and are defined relative to the upper limit of normal (ULN) as follows:

### Aspartate Aminotransferase [AST] Increased

$$> ULN - 3. \times ULN \qquad Grade 1$$

$$> 3. \times ULN - 5. \times ULN \qquad Grade 2$$

$$> 5. \times ULN - 20. \times ULN \qquad Grade 3$$

$$> 20. \times ULN \qquad Grade 4$$

### Alanine Aminotransferase [ALT] Increased

$$> ULN - 3. \times ULN \qquad Grade 1$$

$$> 3. \times ULN - 5. \times ULN \qquad Grade 2$$

$$> 5. \times ULN - 20. \times ULN \qquad Grade 3$$

$$> 20. \times ULN \qquad Grade 4$$

### Alkaline Phosphatase [ALP] Increased

$$> ULN - 2.5 \times ULN \qquad Grade 1$$

$$> 2.5 \times ULN - 5. \times ULN \qquad Grade 2$$

$$> 5. \times ULN - 20. \times ULN \qquad Grade 3$$

$$> 20. \times ULN \qquad Grade 4$$

### Blood Bilirubin Increased

$$> ULN - 1.5 \times ULN \qquad Grade 1$$

$$> 1.5 \times ULN - 3. \times ULN \qquad Grade 2$$

$$> 3. \times ULN - 10. \times ULN \qquad Grade 3$$

$$> 10. \times ULN \qquad Grade 4$$

### Disease Specific Assessments

(1) Tender and swollen joint count: 44 joint count. (2) Subject global health assessment. (3) Health care provider global health assessment. (4) Visual analog pain scale. (5) Health Assessment Questionnaire—Disability Index (HAQ-DI, see FIG. 24). (6) PROMIS-29 Profile, see FIG. 25. (7) RAPID3, see FIG. 23A and FIG. 23B. (8) Pain assessment. (9) Patient global health assessment.

### Mechanistic Assessments

The following samples are collected for mechanistic assessments: Clinic collection: Plasma PK, anti-drug antibody, and sCD40L assays, Serum PK assays, Peripheral blood mononuclear cells (PBMCs), Serum, Whole blood DNA, Whole blood RNA, Whole blood RNA-microcontainer. Home collection after week 16 and through week 40 by subject: Whole blood RNA-microcontainer.

### Mechanistic Assays

Serial blood specimens are collected to interrogate mechanisms of tolerance induction and maintenance. The objectives are to determine how the addition of VIB4920 to TNF-α inhibition affects the frequencies, phenotypes, and functional profiles of relevant T cell, B cell and myeloid cell populations in blood, and to quantify soluble mediators in serum and plasma associated with RA and the blockade of CD40:CD40L signaling. These studies explore immune signatures that correlate with clinical response outcomes.

### Peripheral Blood Cells

Flow cytometry, mass cytometry, CITE-seq, single cell or bulk RNAseq, ATACseq, and DNA methylation sequencing are done at ITN laboratories to determine how the addition of VIB4920 to a TNFi affects the frequency, phenotype, gene expression, and functional status of specific immune cell populations in viably cryopreserved PBMC. In addition, functional status of various cell subsets may be examined by in vitro culture. To investigate oligoclonality of purified T cells and B cells, DNA and/or RNA encoding the T cell receptor and B cell receptor, respectively are sequenced. Profiles of circulating cells are compared between the baseline and various time points following treatment.

Additional comparisons may be made between treatment groups to evaluate the effect of treatment on specific cell phenotypes and profiles and to identify phenotypes and/or profiles that correlate with clinical outcomes. The following cellular parameters may be interrogated to determine the effects of VIB4920 or placebo in RA subjects that have responded inadequately to TNF-α inhibition:
1. Frequency, phenotype, gene expression, and functional profile of CD4+CD27-HLA-DR+, CD4+CXCR5-PD1hi Tph, Tfh, or other T cells in blood.
2. Frequency, phenotype, gene expression, and functional profile of CD14+myeloid subsets in blood.
3. Frequency, phenotype, gene expression, and functional profile of ABCs, memory B cells, and other B cell subsets (i.e. CD21-/low) in blood.
4. Expression level and frequency of CD40 and CD40L proteins in relevant myeloid, B cell and T cell populations in blood.
5. Oligoclonality of T cells and B cells in blood, as measured by T cell receptor and B cell receptor sequencing.
6. Epigenetic profile of purified cell subsets, such as T cells and B cells.
7. Frequency, phenotype, gene expression, and functional profile of other relevant hematopoietic or non-hematopoietic cells in blood.

### Serum and Plasma Assays

Subject serum and plasma are collected and stored for longitudinal analyses using validated platforms to determine how addition of VIB4920 or VIB4920 placebo affects RA in subjects that have responded inadequately to TNFi therapy. Soluble factors that may be examined include: (1) Levels of antibodies or autoantibodies, such as rheumatoid factor and anti-cyclic citrullinated peptide antibody (ACPA). (2) Levels of sCD40L. (3) Levels of anti-drug antibodies targeting adalimumab, etanercept, and VIB4920. (3) Levels of VIB4920. (4) Levels of adalimumab or etanercept as applicable. (5) Levels of CXCL13, sICAM, and any other relevant soluble mediators (e.g., IL-21, IL-10, IL-2, IL-17A, IFNγ, etc.).

Levels of soluble immune parameters may be compared between the baseline and time points following treatment. Comparisons may be made between treatment groups to evaluate the effect of the therapeutic intervention on changes in circulating levels of soluble immune parameters. In addition, the relationship between baseline sICAM and CXCL13 levels and treatment response is determined. The impact of coadministration of TNFi and VIB4920 on the drug levels of VIB4920 and adalimumab or etanercept may be assessed using parameters such as AUC, Cₘₐₓ, and t_{1/2} (half-life). Finally, levels of soluble immune parameters may also be evaluated for associations with frequency, phenotype, and/or functional profile of circulating cells, such as T cell, B cell and myeloid populations

### Whole Blood Assays

RNA: Systemic treatment with biologic medications has been shown to modulate gene expression in autoimmune disease; therefore, whole blood can be used to evaluate changes in the transcriptional signatures of circulating immune cells related to the experimental intervention, namely VI1B4920. Whole blood is collected and may be used to evaluate gene expression profiles before, during, and after treatment. Gene expression of molecules found to be modulated by treatment in blood leukocytes may be investigated in whole blood using quantitative methods.

In the current study, whole blood is collected in clinic at week 16 and then weekly at home (“dense sampling collection”) from week 17 through week 40 by subjects who achieve the primary endpoint at week 16 (SDAI≤11). These samples are used to examine gene expression signatures of various cell types and their changes over time, as well as to explore whether the circulating fibroblast and activated B cell gene signatures detected shortly before a disease flare as assessed by RAPID3.

DNA: Specific CD40 alleles are associated with RA risk and B cells homozygous for the CD40 risk allele display increased surface expression of CD40 compared to their non-risk allele counterparts. Thus, genetic differences may, in part, determine response to CD40L blockade with V1B4920. DNA is collected from all consenting subjects, and ITN may perform genotyping or sequencing of relevant disease- or immune-associated genes, such as HLA Class I/II alleles, genes with reported associations to RA, or genes related to the CD40L-CD40 pathway to investigate correlations with disease activity and therapeutic response. Similarly, whole blood may be used to examine epigenetic status of relevant disease- or immune-associated genes and to investigate relationships between epigenetics and disease activity or therapeutic response.

TABLE 22
Schedule of events: screening and study drug administration

| | | STUDY DRUG ADMINISTRATION | | | | | | | | | |
|---|---|---|---|---|---|---|---|---|---|---|---|
| | | Week | | | | | | | | | |
| | Screening | 0 | 2 | 4 | 6 | 8 | 10 | 12 | 14 | | |
| | Visit | | | | | | | | | | |
| Phase of trial | −1 | 0 | 1 | 2 | 3 | 4 | 5 | 6 | 7 | U¹ | TM/DV² |

GENERAL ASSESSMENTS

| | | | | | | | | | | | |
|---|---|---|---|---|---|---|---|---|---|---|---|
| Informed Consent | X | | | | | | | | | | |
| Eligibility criteria | X | | | | | | | | | | |
| Demographics (age, gender, ethnicity) | X | | | | | | | | | | |
| Medical history | X | | | | | | | | | | |
| Rheumatoid arthritis history | X | | | | | | | | | | |
| Comprehensive physical exam | X | X | | | | | | | | X | X |
| Vital signs | X | X³ | X³ | X³ | | X³ | | X³ | | X | X |
| Randomization | | X | | | | | | | | | |
| Limited physical exam | | | X | X | | X | | X | | | |
| Adverse events | | X | X | X | X | X | X | X | X | X | X |
| Concomitant medications | X | X | X | X | X | X | X | X | X | X | X |
| Telephone assessment of changes since | | | | | X | | X | | X | | |
| prior visit | | | | | | | | | | | |

DISEASE SPECIFIC ASSESSMENTS

| | | | | | | | | |
|---|---|---|---|---|---|---|---|---|
| Tender and swollen joint count4 | X | X | X | X | X | X | X | X |
| Patient global health assessment (PaGH) | X | X | X | X | X | X | X | X |
| Health care provider global health | X | X | X | X | X | X | X | X |
| assessment (PrGH)⁴ | | | | | | | | |
| Pain assessment | | X | | | | | X | X |
| Health Assessment Questionnaire- | | X | | | | | X | X |
| Disability Index (HAQ-DI) | | | | | | | | |
| PROMIS-29 Profile | | X | | | | | X | X |

STUDY DRUG ADMINISTRATION

| | | | | | |
|---|---|---|---|---|---|
| VIB4920 or VIB4920 placebo | X | X | X | X | X |

CLINICAL LABORATORY ASSESSMENTS (Central Lab)

| | | | | | | | | |
|---|---|---|---|---|---|---|---|---|
| Hematology (CBC, differential, and | X | X | | X | X | X | X | X |
| platelet count) | | | | | | | | |
| Serum chemistry (AST, ALT, bilirubin, | X | X | | X | X | X | X | X |
| alkaline phosphatase, albumin, | | | | | | | | |
| creatinine) | | | | | | | | |
| Inflammatory markers (erythrocyte | X | X | X | X | X | X | X | X |
| sedimentation rate, C-reactive protein) | | | | | | | | |
| HIV (RNA or antibody) | X | | | | | | | |
| Hepatitis B (core antibody, surface | X | | | | | | | |
| antigen) | | | | | | | | |
| Hepatitis C (RNA or antibody) | X | | | | | | | |
| Tuberculosis Testing-QuantiFERON- | X | | | | | | | |
| TB Gold, QuantiFERON-TB Gold Plus, | | | | | | | | |
| or T-SPOT.TB test⁵ | | | | | | | | |
| Anti-phospholipid antibody testing (anti- | X | | | | | | | |
| cardiolipin IgG, IgM, and IgA; anti-beta- | | | | | | | | |
| 2-glycoprotein I IgG, IgM, and IgA; | | | | | | | | |
| lupus anticoagulant) | | | | | | | | |
| SARS-COV-2 PCR test | X | | | | | | | |
| Serum pregnancy⁶ | X | | | | | | | |
| Rheumatoid Factor (RF) | X | | | | | | X | X |
| Anti-citrullinated peptide antibodies | X | | | | | | | |
| (ACPA) | | | | | | | | |
| STAT urine pregnancy⁶ | | X | X | X | X | X | X | X |

MECHANISTIC ASSESSMENTS

| | | | | | | |
|---|---|---|---|---|---|---|
| Plasma PK pre-infusion assays⁷ | X | X | X | X | X | |
| Serum PK pre-infusion assays⁷ | X | X | X | X | X | |
| Plasma PK post-infusion ⁷ | X | | | | | |
| Plasma anti-drug antibody and sCD40L | X | X | X | X | X | |
| Assays8 | | | | | | |
| PBMCs⁸ | X | | X | | X | X |
| Serum⁸ | X | | X | | X | X |
| Whole blood RNA⁸ | X | | X | | X | X |
| Whole blood DNA⁸ | X | | X | | X | X |

¹Unscheduled visit
²Treatment Modification or Study Discontinuation visit. If treatment modification or intent to discontinue the study is identified during an in-person visit, convert that visit to a Treatment Modification Visit or Study Discontinuation visit. . If treatment modification or intent to discontinue the study is identified between in-person visits, then schedule the Treatment Modification or Study Discontinuation Visit for the next scheduled in-person visit
³Vitals monitored as described herein
⁴Assessed by blinded evaluator
⁵For participants with indeterminate QuantiFERON-TB Gold, QuantiFERON-TB Gold Plus or T-SPOT.TB tests: 1) PPD skin test, if not performed within the past 3 months; 2) AP and lateral chest radiograph, if not performed within the past 3 months
⁶For women with childbearing potential
⁷Plasma and serum samples for PK collected pre-infusion and within 15 min +/− 5 min minutes post-infusion

TABLE 23
Schedule of events: Post-drug administration with dense sampling collection

| | POST-ADMINISTRATION OBSERVATION WITH DENSE SAMPLING | | | | | | | | | | | | | | |
|---|---|---|---|---|---|---|---|---|---|---|---|---|---|---|---|
| | Week | | | | | | | | | | | | | | |
| | | 17, | | 21, | | 25, | | 29, | | 33, | | 37, | | | |
| | | 18, | | 22, | | 26, | | 30, | | 34, | | 38, | | | |
| | 16 | 19 | 20 | 23 | 24 | 27 | 28 | 31 | 32 | 35 | 36 | 39 | 40 | | |
| | Visit | | | | | | | | | | | | | | |
| | | 8.1, | | 9.1, | | 10.1, | | 11.1, | | 12.1, | | 13.1, | | | |
| | | 8.2, | | 9.2, | | 10.2, | | 11.2, | | 12.2, | | 13.2, | | | |
| Phase of trial | 8 | 8.3 | 9 | 9.3 | 10 | 10.3 | 11 | 11.3 | 12 | 12.3 | 13 | 13.3 | 14 | U⁸ | TMDV⁹ |
| GENERAL ASSESSMENTS | | | | | | | | | | | | | | | |
| Comprehensive physical exam | X | | | | | | | | | | | | X | X | X |
| Vital signs | X | | X | | X | | X | | X | | X | | X | X | X |
| Limited physical exam | | | X | | X | | X | | X | | X | | | | |
| Adverse events | X | | X | | X | | X | | X | | X | | X | X | X |
| Concomitant medications | X | | X | | X | | X | | X | | X | | X | X | X |
| DISEASE SPECIFIC ASSESSMENTS | | | | | | | | | | | | | | | |
| Tender and swollen joint count | X | | X | | X | | X | | X | | X | | X | X | X |
| Patient global health assessment | X | | X | | X | | X | | X | | X | | X | X | X |
| Health care provider global health | X | | X | | X | | X | | X | | X | | X | X | X |
| assessment | | | | | | | | | | | | | | | |
| Pain assessment | X | | | | | | | | | | | | | | |
| RAPID3 assessment (for dense sampling | X | X | X | X | X | X | X | X | X | X | X | X | X | | X |
| collection) | | | | | | | | | | | | | | | |
| Health Assessment Questionnaire | X | | | | | | | | | | | | X | X | X |
| Disability Index (HAQ-DI) | | | | | | | | | | | | | | | |
| PROMIS-29 Profile | X | | | | | | | | | | | | X | X | X |
| CLINICAL LABORATORY | | | | | | | | | | | | | | | |
| ASSESSMENTS | | | | | | | | | | | | | | | |
| Hematology (CBC, differential, and | X | | | | X | | | | X | | | | X | X | X |
| platelet count) | | | | | | | | | | | | | | | |
| Serum chemistry (AST, ALT, bilirubin, | X | | | | X | | | | X | | | | X | X | X |
| alkaline phosphatase, albumin, | | | | | | | | | | | | | | | |
| creatinine) | | | | | | | | | | | | | | | |
| Inflammatory markers (erythrocyte | X | | X | | X | | X | | X | | X | | X | X | X |
| sedimentation rate, C-reactive protein) | | | | | | | | | | | | | | | |
| Rheumatoid Factor (RF) | X | | | | | | | | | | | | X | X | X |
| Anti-citrullinated peptide antibodies | X | | | | | | | | | | | | X | X | X |
| (ACPA) | | | | | | | | | | | | | | | |
| MECHANISTIC ASSESSMENTS | | | | | | | | | | | | | | | |
| Plasma PK Assay | X | | X | | X | | X | | X | | X | | X | | |
| Serum PK Assay | X | | X | | X | | X | | X | | X | | X | | |
| Plasma ADA and sCD40L Assays | X | | X | | X | | X | | X | | X | | X | | |
| PBMCs | X | | | | X | | | | X | | | | X | | X |
| Serum | X | | | | X | | | | X | | | | X | | X |
| Whole blood RNA | X | | | | X | | | | X | | | | X | | X |
| Whole blood RNA-microcontainer | X | | | | | | | | | | | | | | |
| (clinic collection) | | | | | | | | | | | | | | | |
| Whole blood RNA-microcontainer | X | X | X | X | X | X | X | X | X | X | X | X | X | | |
| (home collection for dense sampling | | | | | | | | | | | | | | | |
| collection) | | | | | | | | | | | | | | | |
| Whole blood DNA | X | | | | X | | | | X | | | | X | | X |

⁸Unscheduled visit.
⁹Treatment Modification or Study Discontinuation visit. If treatment modification or intent to discontinue the study is identified during an in person visit, convert that visit to a Treatment Modification Visit or Study Discontinuation visit. If treatment modification or intent to discontinue the study is identified between in-person visits, then schedule the Treatment Modification or Study Discontinuation Visit for the next scheduled in-person visit.

TABLE 24 25
Schedule of events: Post-drug administration without dense sampling collection

| | POST-ADMINISTRATION OBSERVATION | | | | | | | | |
|---|---|---|---|---|---|---|---|---|---|
| | Week | | | | | | | | |
| | 16 | 20 | 24 | 28 | 32 | 36 | 40 | | |
| | Visit | | | | | | | | |
| Phase of trial | 8 | 9 | 10 | 11 | 12 | 13 | 14 | U | TM/DV |

GENERAL ASSESSMENTS

| | | | | | | | | | |
|---|---|---|---|---|---|---|---|---|---|
| Comprehensive physical exam | X | | | | | | X | X | X |
| Vital signs | X | X | X | X | X | X | X | X | X |
| Limited physical exam | | X | X | X | X | X | | | |
| Adverse events | X | X | X | X | X | X | X | X | X |
| Concomitant medications | X | X | X | X | X | X | X | X | X |

DISEASE SPECIFIC ASSESSMENTS

| | | | | | | | | | |
|---|---|---|---|---|---|---|---|---|---|
| Tender and swollen joint count | X | X | X | X | X | X | X | X | X |
| Patient global health assessment | X | X | X | X | X | X | X | X | X |
| Health care provider global health assessment | X | X | X | X | X | X | X | X | X |
| Visual analog pain scale | X | | | | | | | | |
| Health Assessment Questionnaire - Disability Index | X | | | | | | X | X | X |
| (HAQ-DI) | | | | | | | | | |
| Promis-29 Profile | X | | | | | | X | X | X |

CLINICAL LABORATORY ASSESSMENTS

| | | | | | | | | | |
|---|---|---|---|---|---|---|---|---|---|
| Hematology (CBC, differential, and platelet count) | X | | X | | X | | X | X | X |
| Serum chemistry (AST, ALT, bilirubin, alkaline | X | | X | | X | | X | X | X |
| phosphatase, albumin, creatinine) | | | | | | | | | |
| Inflammatory markers (erythrocyte sedimentation | X | X | X | X | X | X | X | X | X |
| rate, C-reactive protein) | | | | | | | | | |
| Rheumatoid Factor (RF) | X | | | | | | X | X | X |
| Anti-citrullinated peptide antibodies (ACPA) | X | | | | | | X | X | X |

MECHANISTIC ASSESSMENTS

| | | | | | | | | |
|---|---|---|---|---|---|---|---|---|
| Plasma PK Assays | X | X | X | X | X | X | X | |
| Serum PK Assays | X | X | X | X | X | X | X | |
| Plasma ADA and sCD40L Assays | X | X | X | X | X | X | X | |
| PBMCs | X | | X | | X | | X | X |
| Serum | X | | X | | X | | X | X |
| Whole blood RNA | X | | X | | X | | X | X |
| Whole blood DNA | X | | X | | X | | X | X |

Schedule of events: Alternate monitoring

| | ALTERNATE MONITORING | | | | | | | | | |
|---|---|---|---|---|---|---|---|---|---|---|
| | Week of Study | | | | | | | | | |
| | 2 | 4 | 8 | 12 | 16 | 24 | 32 | 40 | | |
| | Visit | | | | | | | | | |
| Phase of trial | M1 | M2 | M3 | M4 | M5 | M6 | M7 | M8 | U¹⁰ | DV¹¹ |

GENERAL ASSESSMENTS

| | | | | | | | | | | |
|---|---|---|---|---|---|---|---|---|---|---|
| Limited physical exam | X | X | X | X | X | X | X | X | X | X |
| Adverse events¹² | X | X | X | X | X | X | X | X | X | X |
| Concomitant medications | X | X | X | X | X | X | X | X | X | X |

CLINICAL LABORATORY ASSESSMENTS

| | | | | | | | |
|---|---|---|---|---|---|---|---|
| Hematology (CBC, differential, and platelet count) | X | X | X | X | X | X | X |
| Serum chemistry (AST, ALT, bilirubin, | X | X | X | X | X | X | X |
| alkaline phosphatase, albumin, creatinine) | | | | | | | |
| Inflammatory markers | | | | X | | | |
| (erythrocyte sedimentation rate, C-reactive protein) | | | | | | | |

DISEASE SPECIFIC ASSESSMENTS

| | | | | |
|---|---|---|---|---|
| Tender and swollen joint count | X | X | X | X |
| Patient global health assessment | X | X | X | X |
| Health care provider global health assessment | X | X | X | X |
| Pain assessment | X | | | |

¹⁰Unscheduled visit.
¹¹Study Discontinuation visit. If intent to discontinue the study is identified during an in person visit, convert that visit to a Study Discontinuation visit. If intent to discontinue the study is identified between in-person visits, then schedule the Study Discontinuation Visit for the next scheduled in-person visit.
¹²Adverse event monitoring will assess grade 2 or higher adverse events that receive medical attention and AESIs.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method for treating rheumatoid arthritis in a subject in need thereof comprising:
administering a Tn3 scaffold comprising a CD40L-specific monomer subunit to the subject;
wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16,
wherein the Tn3 scaffold comprising the CD40L-specific monomer subunit is administered at a dose of about 1500 mg, and
wherein the Tn3 scaffold is administered once about every 2 weeks for at least 2 doses, and is administered about once a month thereafter.

2. (canceled)

3. A method for treating rheumatoid arthritis in a subject in need thereof comprising:
administering a Tn3 scaffold comprising a CD40L-specific monomer subunit to the subject;
wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and
wherein the Tn3 scaffold is administered once about every 2 months for at least 2 doses.

4. A method for treating rheumatoid arthritis in a subject in need thereof comprising:
administering a Tn3 scaffold comprising a CD40L-specific monomer subunit to the subject;
wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 3000 mg, and
wherein the Tn3 scaffold is administered about once, once a month, once about every two months, or once about every three months.

5. The method of claim 1, wherein the Tn3 scaffold is administered in combination with a second therapy.

6. The method of claim 5, wherein the second therapy comprises an anti-inflammatory agent, an anti-pain agent, a Disease-modifying Antirheumatic Drug (DMARD), a corticosteroid, or an immune system modifying agent.

7. (canceled)

8. (canceled)

9. (canceled)

10. The method of claim 1, wherein the Tn3 scaffold is administered intravenously.

11. (canceled)

12. The method of claim 1, wherein the Tn3 scaffold comprises two CD40L-specific monomer subunits connected in tandem.

13. The method of claim 1, wherein the Tn3 scaffold binds CD40L and prevents binding of CD40L to CD40 and/or disrupts CD40 mediated signaling.

14. (canceled)

15. The method of claim 1, wherein at least one CD40L-specific monomer subunit is fused to a human serum albumin (HSA).

16. The method of claim 15, wherein the HSA is a variant HSA comprising the amino acid sequence of SEQ ID NO: 4.

17. The method of claim 1, wherein the Tn3 scaffold comprises the sequence of SEQ ID NO: 1.

18. The method of claim 1, wherein the Tn3 scaffold is Dazodalibep.

19. (canceled)

20. (canceled)

21. The method of claim 1, wherein a first dose of the Tn3 scaffold is about 1500 mg and subsequent doses are about 1500 mg or about 3000 mg.

22. The method of claim 21, wherein the first and a second dose of the Tn3 scaffold are about 1500 mg and the subsequent doses are about 3000 mg.

23. (canceled)

24. (canceled)

25. The method of claim 4, wherein the Tn3 scaffold is administered quarterly.

26. (canceled)

27. (canceled)

28. (canceled)

29. (canceled)

30. The method of claim 25, wherein the quarterly administration of the Tn3 scaffold confers sustained treatment efficacy in the subject in need as compared to an otherwise comparable subject undergoing more frequent administrations of the Tn3 scaffold as determined by a treatment assessment, and wherein the sustained treatment efficacy is of at least or at most about 15 days, 1 month, 2 months, 3 months, 4 months, 5 months, 6 months, 8 months, 10 months, 1 year, or 1.5 years.

31. The method of claim 30, wherein the treatment assessment is determined quarterly.

32. The method of claim 1, wherein the CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21.

33. The method of claim 12, wherein the CD40L-specific monomer subunit comprises the polypeptide of SEQ ID NO: 22, and wherein the second CD40L-specific monomer subunit comprises the polypeptide of SEQ ID NO: 25.

34. The method of claim 1, wherein the CD40L-specific monomer subunit comprises the polypeptide of SEQ ID NO: 22

35. The method of claim 1, wherein the CD40L-specific monomer subunit comprises the polypeptide of SEQ ID NO: 25.


SEQUENCE LISTING (25 sequences)

SEQ ID NO: 1 - protein, 785 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 83 aa, synthetic construct
IEVKDVTDTTALITWSDDFGEYVWCELTYGIKDVPGDRTTIDLWYHHAHYSIGNLKPDTEYEVSLICRRGDMSSNPAKETFTT

SEQ ID NO: 3 - protein, 83 aa, synthetic construct
IEVKDVTDTTALITWSDDFGEYVWCELTYGIKDVPGDRTTIDLWYHHAHYSIGNLKPDTEYEVSLICRSGDMSSNPAKETFTT

SEQ ID NO: 4 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - skipped

SEQ ID NO: 6 - protein, 5 aa, synthetic construct
ALITW

SEQ ID NO: 7 - protein, 10 aa, synthetic construct
  REGION 1..10: note = linker
GGGGSGGGGS

SEQ ID NO: 8 - protein, 15 aa, synthetic construct
  REGION 1..15: note = linker
GGGGSGGGGSGGGGS

SEQ ID NO: 9 - protein, 10 aa, synthetic construct
  REGION 1..10: note = linker
GGGGGGGGGG

SEQ ID NO: 10 - protein, 15 aa, synthetic construct
  REGION 1..15: note = linker
GGGGGGGGGGGGGGG

SEQ ID NO: 11 - protein, 7 aa, synthetic construct
KDVTDTT

SEQ ID NO: 12 - protein, 9 aa, synthetic construct
SDDFGEYVW

SEQ ID NO: 13 - protein, 7 aa, synthetic construct
KDVPGDR

SEQ ID NO: 14 - protein, 6 aa, synthetic construct
WYHHAH

SEQ ID NO: 15 - protein, 8 aa, synthetic construct
GNLKPDTE

SEQ ID NO: 16 - protein, 10 aa, synthetic construct
RSGDMSSNPA

SEQ ID NO: 17 - protein, 7 aa, synthetic construct
CELTYGI

SEQ ID NO: 18 - protein, 5 aa, synthetic construct
TTIDL

SEQ ID NO: 19 - skipped

SEQ ID NO: 20 - protein, 7 aa, synthetic construct
YEVSLIC

SEQ ID NO: 21 - protein, 6 aa, synthetic construct
KETFTT

SEQ ID NO: 22 - protein, 85 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 23 - protein, 10 aa, synthetic construct
RLDAPSQIEV

SEQ ID NO: 24 - protein, 5 aa, synthetic construct
SQIEV

SEQ ID NO: 25 - protein, 90 aa, synthetic construct
RLDAPSQIEVKDVTDTTALITWSDDFGEYVWCELTYGIKDVPGDRTTIDLWYHHAHYSIGNLKPDTEYEVSLICRSGDMSSNPAKETFTT